SEQUENCE LISTING (9 sequences)

SEQ ID NO: 1 - protein, 200 aa, Hepatitis C virus
SGSWLRDVWDWICTVLTDFKTWLQSKLLPRLPGVPFFSCQRGYKGVWRGDGIMQTTCPCGAQITGHVKNGSMRIVGPRTCSNTWHGTFPINAYTTGPCTPSPAPNYSRALWRVAAEEYVEVTRVGDFHYVTGMTTDNVKCPCQVPAPEFFTEVDGVRLHRYAPACKPLLREEVTFLVGLNQYLVGSQLPCEPEPDVAVLT

SEQ ID NO: 2 - protein, 200 aa, Hepatitis C virus
SGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLT

SEQ ID NO: 3 - protein, 200 aa, Hepatitis C virus
GGSWLRDVWDWVCTILTDFKNWLTSKLFPKMPGLPFVSCQKGYKGVWAGTGIMTTRCPCGANISGNVRLGSMRITGPKTCMNIWQGTFPINCYTEGQCVPKPAPNFKVAIWRVAASEYAEVTQHGSYHYITGLTTDNLKVPCQLPSPEFFSWVDGVQIHRFAPTPKPFFRDEVSFCVGLNSFVVGSQLPCDPEPDTDVLM

SEQ ID NO: 4 - protein, 200 aa, Hepatitis C virus
SGSWLRDVWDWVCSILTDFKNWLSSKLLPKMPGLPFISCQKGYRGVWAGTGVMTTRCPCGANISGHVRMGTMKITGPKTCLNLWQGTFPINCYTEGPCVPKPPPNYKTAIWRVAASEYVEVTQHGSFSYVTGLTSDNLKVPCQVPAPEFFSWVDGVQIHRFAPTPGPFFRDEVTFTVGLNSFVVGSQLPCDPEPDTEVLA

SEQ ID NO: 5 - protein, 200 aa, Hepatitis C virus
SDDWLRIIWDWVCSVLSDFKSWLSAKIMPALPGLPFISCQKGYKGVWRGDGVMSTRCPCGATITGHVKNGSMRLAGPRTCANMWHGTFPINEYTTGPSTPCPSPNYTRALWRVAANSYVEVRRVGDFHYITGATEDELKCPCQVPAAEFFTEVDGVRLHRYAPPCKPLLREEITFSVGLNSYTIGSQLPCEPEPDVSVLT

SEQ ID NO: 6 - protein, 200 aa, Hepatitis C virus
ADSWLWEVWDWVCTVLSDFKTWLKAKLMPLMPGIPFLSCQRGYKGEWRGDGVMHTTCPCGAELAGHIKNGSMRITGPKTCSNTWHGTFPINAYTTGPGVPVPAPNYKFALWRVSAEEYVEVRRVGDFHYVTGVTQDNIKCPCQVPAPEFFTEVDGIRLHRHAPKCKPLLRDEVSFSVGLNSFVVGSQLPCEPEPDVAVLT

SEQ ID NO: 7 - protein, 200 aa, Hepatitis C virus
DGTWLRAIWDWVCTALTDFKAWLQAKLLPQLPGVPFFSCQKGYKGVWRGDGVNSTKCPCGATISGHVKNGTMRIVGPKLCSNTWQGTFPINATTTGPSVPAPAPNYKFALWRVGAADYAEVRRVGDYHYITGVTQDNLKCPCQVPSPEFFTELDGVRIHRFAPPCNPLLREEVTFSVGLHYVVGSQLPCEPEPDVTVLTS

SEQ ID NO: 8 - protein, 200 aa, Hepatitis C virus
ATSWLRDVWDWVCTVLSDFKVWLQAKLLPRLPGIPFLSCQTGYRGVWAGDGVCHTTCTCGAVIAGHVKNGSMKITGPKTCSNTWHGTFPINATTTGPSTPRPAPSYQRALWRVSAEDYVEVRRLGDCHYVVGVTADGLKCPCQVPAPEFFTEVDGVRIHRYAPPCKPLLRDEVTFSVGLSNYAIGSQLPCEPEPDVTIVT

SEQ ID NO: 9 - protein, 447 aa, Hepatitis C virus
SGSWLRDVWDWICTVLTDFKTWLQSKLLPRLPGVPFFSCQRGYKGVWRGDGIMQTTCPCGAQITGHVKNGSMRIVGPRTCSNTWHGTFPINAYTTGPCTPSPAPNYSRALWRVAAEEYVEVTRVGDFHYVTGMTTDNVKCPCQVPAPEFFTEVDGVRLHRYAPACKPLLREEVTFLVGLNQYLVGSQLPCEPEPDVAVLTSMLTDPSHITAETAKRRLARGSPPSLASSSASQLSAPSLKATCTTRHDSPDADLIEANLLWRQEMGGNITRVESENKVVILDSFEPLQAEEDEREVSVPAEILRRSRKFPRAMPIWARPDYNPPLLESWKDPDYVPPVVHGCPLPPAKAPPIPPPRRKRTVVLSESTVSSALAELATKTFGSSESSAVDSGTATASPDQPSDDGDAGSDVESYSSMPPLEGEPGDPDLSDGSWSTVSEEASEDVVCC

(19) United States
(12) Patent Application Publication
(10) Pub. No.: US 2011/0137633 A1
(43) Pub. Date: Jun. 9, 2011

(54) ANTI-VIRAL COMPOUNDS AND METHODS OF IDENTIFYING THE SAME

(75) Inventors: CHARLES W. HUTCHINS, GREEN OAKS, IL (US); DAVID A. DEGOEY, SALEM, WI (US); WARREN M. KATI, GURNEE, IL (US); ALLAN C. KRUEGER, GURNEE, IL (US); JOHN T. RANDOLPH, LIBERTYVILLE, IL (US); ROLF WAGNER, ANTIOCH, IL (US); PAMELA L. DONNER, MUNDELEIN, IL (US)

(73) Assignee: ABBOTT LABORATORIES, ABBOTT PARK, IL (US)

(21) Appl. No.: 12/959,941

(22) Filed: Dec. 3, 2010

Related U.S. Application Data
(60) Provisional application No. 61/266,333, filed on Dec. 3, 2009.

(51) Int. Cl. G06G 7/48; C07D 403/12; C07D 403/14; C07D 409/14; C07D 401/14

(52) U.S. Cl. 703/11; 548/518; 548/313.1; 548/311.4; 546/158

(57) ABSTRACT

Methods of identifying NS5A inhibitors are provided. Novel binding sites are identified on NS5A dimers, and these binding sites can be used to design and/or identify new NS5A inhibitors.

This application claims benefit from and incorporates by reference the entire content of U.S. Provisional Application No. 61/266,333, filed Dec. 3, 2009. This application also incorporates by reference all material in the ASCII text file submitted herewith via EFS-Web. The ASCII text file is entitled “Sequence Listing.txt”, has the size of 18.0 KB, and was created on Dec. 2, 2009.

### FIELD

The present invention relates to anti-HCV compounds and methods of identifying the same.

### BACKGROUND

Hepatitis C virus (“HCV”) is an RNA virus belonging to the Hepacivirus genus in the Flaviviridae family. The enveloped HCV virion contains a positive stranded RNA genome which encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often inadequate. Therefore, there is a need for new drugs to treat HCV infection.

The nonstructural protein NS5A is a membrane-associated phosphoprotein present in basally phosphorylated and hyperphosphorylated forms. It is a critical component of HCV replication and is believed to exert multiple functions at various stages of the viral life cycle. A full-length NS5A protein typically has 447 amino acid residues and comprises three domains—namely, Domain I, Domain II, and Domain III. Domain I (residues 1 to 213) contains a zinc-binding motif and an amphipathic N-terminal helix which can promote membrane association. Domain II (residues 250 to 342) has regulatory functions, such as interactions with protein kinase PKR and PI3K, as well as NS5B, and also contains the interferon sensitivity-determining region. Domain III (residues 356 to 447) plays a role in infectious virion assembly, and can be modulated by phosphorylation within the domain.

NS5A has been identified as a promising therapeutic target for treating HCV. However, designing new NS5A inhibitors has been hampered by a lack of understanding of the structure-activity relationship between the target and the inhibitors. Tellinghuisen et al., NATURE 435:374-379 (2005), described a crystal structure of the NS5A protein at about 2.5 angstroms resolution. This structure reveals two NS5A monomers packed as a dimer via contacts near the N-terminal ends of the molecules. However, the structural complexity and the versatile functions of NS5A have made it difficult to find the inhibitor binding sites on the NS5A protein.

### SUMMARY

The present invention features the identification of unexpected binding sites on NS5A dimers. Compounds that fit to these binding sites showed significant anti-HCV activities. Accordingly, these binding sites can be used to design and/or identify new NS5A inhibitors.

In one aspect, the present invention features a method of identifying NS5A inhibitors, said method comprising docking a compound to an NS5A dimer using a computer docking program, wherein the NS5A dimer comprises two NS5A monomers and an interaction site, and the amino acid residues that form the interaction site comprise amino acids 37, 38, 39 and 58 of both monomers, and wherein whether the compound comprises a moiety that fits to the interaction site is indicative of whether the compound is an NS5A inhibitor. Preferably, the amino acid residues that form the interaction site comprise amino acids 37, 38, 39, 40 and 58 of both monomers. More preferably, the amino acid residues that form the interaction site comprise Phe 37, Ser 38, Cys 39, Gln 40 and Pro 58 of both monomers.

In another aspect, the present invention features another method of identifying NS5A inhibitors, said method comprising docking a compound to an NS5A dimer using a computer docking program, wherein the NS5A dimer comprises two NS5A monomers and an interaction site, and the amino acid residues that form the interaction site comprise amino acids 81, 85, 110, 152, 155, 165, and 168 of one monomer, and wherein whether at least part of the compound fits to the interaction site is indicative of whether the compound is an NS5A inhibitor. Preferably, the amino acid residues that form the interaction site comprise amino acids 41, 42, 81, 82, 85, 87, 110, 118, 151, 152, 155, 156, 157, 162, 163, 164, 165, 166 and 168 of one monomer. More preferably, the amino acid residues that form the interaction site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of one monomer.

In yet another aspect, the present invention features yet another method of identifying NS5A inhibitors, said method comprising docking a compound to an NS5A dimer using a computer docking program, wherein the NS5A dimer comprises two NS5A monomers and an interaction site, and the amino acid residues that form the interaction site comprise amino acids 81, 85, 110, 152, 155, 165, and 168 of both monomers, and wherein whether at least part of the compound fits to the interaction site is indicative of whether the compound is an NS5A inhibitor. Preferably, the amino acid residues that form the interaction site comprise amino acids 41, 42, 81, 82, 85, 87, 110, 118, 151, 152, 155, 156, 157, 162, 163, 164, 165, 166 and 168 of both monomers. More preferably, the amino acid residues that form the interaction site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of both monomers.

In still another aspect, the present invention features still another method of identifying NS5A inhibitors, said method comprising docking a compound to an NS5A dimer using a computer docking program, wherein the NS5A dimer comprises two NS5A monomers and an interaction site, and the amino acid residues that form the interaction site comprise amino acids 81, 85, 110, 152, 155, 165, and 168 of one monomer, and amino acids 37, 38, 39, and 58 of both monomers, and wherein whether at least part of the compound fits to the interaction site is indicative of whether the compound is an NS5A inhibitor. Preferably, the amino acid residues that form the interaction site comprise amino acids 41, 42, 81, 82, 85, 87, 110, 118, 151, 152, 155, 156, 157, 162, 163, 164, 165, 166 and 168 of one monomer, and amino acids 37, 38, 39, 40 and 58 of both monomers. More preferably, the amino acid residues that form the interaction site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of one monomer, and Phe 37, Ser 38, Cys 39, Gln 40 and Pro 58 of both monomers.

In a further aspect, the present invention features another method of identifying NS5A inhibitors, said method comprising docking a compound to an NS5A dimer using a computer docking program, wherein the NS5A dimer comprises two NS5A monomers and an interaction site, and the amino acid residues that form the interaction site comprise amino acids 37, 38, 39, 58, 81, 85, 110, 152, 155, 165, and 168 of both monomers, and wherein whether at least part of the compound fits to the interaction site is indicative of whether the compound is an NS5A inhibitor. Preferably, the amino acid residues that form the interaction site comprise amino acids 37, 38, 39, 40, 41, 42, 58, 81, 82, 85, 87, 110, 118, 151, 152, 155, 156, 157, 162, 163, 164, 165, 166, and 168 of both monomers. More preferably, the amino acid residues that form the interaction site comprise Phe 37, Ser 38, Cys 39, Gln 40, Arg 41, Gly 42, Pro 58, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of both monomers.

The present invention also features compounds identified according to a method of the present invention.

In addition, the present invention features compounds capable of being docked to an NS5A dimer using a computer docking program, wherein the NS5A dimer comprises two NS5A monomers and an interaction site, and the amino acid residues that form the interaction site comprise amino acids 37, 38, 39 and 58 of both monomers, and wherein the compound comprises a moiety that fits to the interaction site. Preferably, the amino acid residues that form the interaction site comprise amino acids 37, 38, 39, 40 and 58 of both monomers. More preferably, the amino acid residues that form the interaction site comprise Phe 37, Ser 38, Cys 39, Gln 40 and Pro 58 of both monomers.

The moiety that fits to the interaction site can comprise, without limitation, -L-E, wherein:
E is C₃-C₁₄carbocycle or 3- to 14-membered heterocycle, and is optionally substituted with one or more R_{A}; or E is -L_{S}-R_{E};
L is -L_{S}-, -L_{S}-O-L_{S}′-, -L_{S}-C(O)-L_{S}′-, -L_{S}-S(O)₂-L_{S}′-, -L_{S}-S(O)-L_{S}′-, -L_{S}-OS(O)₂-L_{S}′-, L_{S}-S(O)₂O-L_{S}′-, -L_{S}-OS(O)-L_{S}′-, -L_{S}-S(O)O-L_{S}′-, -L_{S}-C(O)O-L_{S}′-, -L_{S}-OC(O)-L_{S}′-, -L_{S}-OC(O)O-L_{S}′-, -L_{S}-C(O)N(R_{B})-L_{S}′-, -L_{S}-N(R_{B})C(O)-L_{S}′-, -L_{S}-C(O)N(R_{B})O-L_{S}′-, -L_{S}-N(R_{B})C(O)O-L_{S}′-, -L_{S}-OC(O)N(R_{B})-L_{S}′-, -L_{S}-C(O)N(R_{B})N(R_{B}′)-L_{S}′-, -L_{S}-S-L_{S}′-, -L_{S}-C(S)-L_{S}′-, -L_{S}-C(S)O-L_{S}′-, -L_{S}-OC(S)-L_{S}′-, -L_{S}-C(S)N(R_{B})-L_{S}′-, -L_{S}-N(R_{B})-L_{S}′-, -L_{S}-N(R_{B})C(S)-L_{S}′-, -L_{S}-N(R_{B})S(O)-L_{S}′-, -L_{S}-N(R_{B})S(O)₂-L_{S}′-, -L_{S}-S(O)₂N(R_{B})-L_{S}′-, -L_{S}-S(O)N(R_{B})-L_{S}′-, -L_{S}-C(S)N(R_{B})O-L_{S}′-, -L_{S}-C(O)N(R_{B})C(O)-L_{S}′-, -L_{S}-N(R_{B})C(O)N(R_{B}′)-L_{S}′-, -L_{S}-N(R_{B})SO₂N(R_{B}′)-L_{S}′-, -L_{S}-N(R_{B})S(O)N(R_{B}′)-L_{S}′-, or -L_{S}-C(S)N(R_{B})N(R_{B}′)-L_{S}′-;
L_{S} and L_{S}′ are each independently selected at each occurrence from bond; or C₁-C₆alkylene, C₂-C₆alkenylene or C₂-C₆alkynylene, each of which is independently optionally substituted at each occurrence with one or more R_{L};
R_{A} is independently selected at each occurrence from halogen, oxo, thioxo, hydroxy, mercapto, nitro, cyano, amino, carboxy, formyl, phosphonoxy, or phosphono; or -L_{S}-R_{E};
R_{B} and R_{B}′ are each independently selected at each occurrence from hydrogen; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆carbocycle or 3- to 6-membered heterocycle; or C₃-C₆carbocycle or 3- to 6-membered heterocycle; wherein each C₃-C₆carbocycle or 3- to 6-membered heterocycle in R_{B} or R_{B}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′) or —C(O)N(R_{S})C(O)—R_{S}′; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{L} is independently selected at each occurrence from halogen, nitro, oxo, phosphonoxy, phosphono, thioxo, cyano, —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′) or —N(R_{S})C(O)R_{S}′; or C₃-C₆carbocycle 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{S}, R_{S}′ and R_{S}″ are each independently selected at each occurrence from hydrogen; C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano or 3- to 6-membered carbocycle or heterocycle; or 3- to 6-membered carbocycle or heterocycle; wherein each 3- to 6-membered carbocycle or heterocycle in R_{S}, R_{S}′ or R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Preferably, the moiety that fits to the interaction site comprises C₅-C₆carbocycle, 5- to 6-membered heterocycle, or 6- to 12-membered bicycle, each of which is optionally substituted with one or more R_{A} as defined above. Also preferably, the moiety comprises C₁-C₆alkyl, C₂-C₆alkenyl or C₂-C₆alkynyl, each of which is optionally substituted with one or more R_{L} as defined above. In one example, the moiety comprises phenyl optionally substituted with one or more substituents selected from is halogen, hydroxy, mercapto, amino, carboxy, C₁-C₆alkyl, C₂-C₆alkenyl or C₂-C₆alkynyl, wherein each of said C₁-C₆alkyl, C₂-C₆alkenyl or C₂-C₆alkynyl is independently optionally substituted at each occurrence with one or more substituents selected from halogen, hydroxy, mercapto, amino or carboxy. In another example, the moiety comprises C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl or cyano. Computer docking programs suitable for the present invention include, but are not limited to, Insight II, FlexX, GRAMM, GRID, MCSS, AUTODOCK, DOCK, GOLD, and ICM. Preferably, Insight II, AUTODOCK or DOCK is used in the present invention to determine if a compound of interest can be docked to an NS5A dimer.

The NS5A dimers employed in the present invention can be of any HCV genotype or subgenotype. The NS5A dimers with HCV consensus sequences can also be used. Preferably, each monomer in an NS5A dimer employed in the present invention comprises an NS5A sequence of HCV 1 (e.g., 1a, 1b, or 1c), HCV 2 (e.g., 2a, 2b, or 2c), HCV 3 (e.g., 3a or 3b), HCV 4 (e.g., 4a, 4b, 4c, 4d, or 4e), HCV 5 (e.g., 5a), HCV 6 (e.g., 6a), HCV 7 (e.g., 7a or 7b), HCV 8 (e.g., 8a or 8b), HCV 9 (e.g., 9a), HCV 10 (e.g., 10a), or HCV 11 (e.g., 11a). More preferably, each monomer employed in the present invention comprises amino acids 37-168 of SEQ ID NO: 1, 2, 3, 4, 5, 6, 7, or 8. Also preferably, each monomer employed in the present invention comprises amino acids 36-198 of SEQ ID NO: 1, 2, 3, 4, 5, 6 or 8 or amino acids 36-197 of SEQ ID NO:7. More preferably, each monomer employed in the present invention consists of amino acids 37-168 of SEQ ID NO: 1, 2, 3, 4, 5, 6, 7, or 8, or amino acids 36-198 of SEQ ID NO: 1, 2, 3, 4, 5, 6 or 8 or amino acids 36-197 of SEQ ID NO:7. Highly preferably, each monomer in an NS5A dimer employed in the present invention comprises amino acids 36-198 of SEQ ID NO: 1. Most preferably, each monomer in an NS5A dimer employed in the present invention consists of amino acids 36-198 of SEQ ID NO: 1.

In one embodiment, each monomer in an NS5A dimer employed in the present invention has at least 50% sequence identity to amino acid 36-198 of SEQ ID NO:1. More preferably, each monomer has at least 75% sequence identity to amino acid 36-198 of SEQ ID NO:1. Highly preferably, each monomer has at least 90% sequence identity to amino acid 36-198 of SEQ ID NO:1. Mostly preferably, each monomer has at least 95% sequence identity to amino acid 36-198 of SEQ ID NO:1.

Preferably, the NS5A dimer employed in the present invention is the 1ZH1 dimer.

The present invention also features compositions comprising the compounds or salts of the present invention. The compositions can also include other therapeutic agents, such as HCV helicase inhibitors, HCV polymerase inhibitors, HCV protease inhibitors, other HCV NS5A inhibitors, CD81 inhibitors, cyclophilin inhibitors, or internal ribosome entry site (IRES) inhibitors.

The present invention further features methods of using the compounds or salts of the present invention to inhibit HCV replication. The methods comprise contacting cells infected with HCV virus with a compound or salt of the present invention, thereby inhibiting the replication of HCV virus in the cells.

In addition, the present invention features methods of using the compounds or salts of the present invention, or compositions comprising the same, to treat HCV infection. The methods comprise administering a compound or salt of the present invention, or a pharmaceutical composition comprising the same, to a patient in need thereof, thereby reducing the blood or tissue level of HCV virus in the patient.

The present invention also features use of the compounds or salts of the present invention for the manufacture of medicaments for the treatment of HCV infection.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The drawings are provided for illustration, not limitation.

FIG. 1A is a computer-generated, ribbon diagram showing a schematic, three-dimensional view of the structure of an NS5A dimer. The solvent-accessible molecular surface near the contact area between the two monomers is also illustrated. The two monomers are depicted in different colors, one in orange and the other in green. The arrow indicates a binding pocket formed by Phe 37, Ser 38, Cys 39, Gln 40 and Pro 58 of both monomers.

FIG. 1B is a rotational view of the molecule surface depicted in FIG. 1A, where the arrow indicates the binding pocket formed by Phe 37, Ser 38, Cys 39, Gln 40 and Pro 58 of both monomers.

FIG. 2A shows a compound docked to an NS5A dimer. The docking pose was generated using Insight II (Accelrys, San Diego, Calif.). The carbon, nitrogen, oxygen, and sulfur atoms in the compound are depicted in cyan, blue, red, and yellow, respectively. The solvent-accessible molecular surface of the NS5A dimer is depicted in two colors, the orange area corresponding to the surface formed by one monomer and the green area corresponding the surface formed by the other monomer. The compound in FIG. 2A lacks a moiety that fits to the binding pocket formed by Phe 37, Ser 38, Cys 39, Gln 40 and Pro 58 of both monomers.

FIG. 2B illustrates another compound docked to the NS5A surface depicted in FIG. 2A. The docking pose was generated using Insight II (Accelrys, San Diego, Calif.). The carbon, nitrogen, oxygen, and sulfur atoms in the compound are depicted in purple, blue, red, and yellow, respectively. The solvent-accessible molecular surface of the NS5A dimer is depicted in two colors, the orange area corresponding to the surface formed by one monomer and the green area corresponding the surface formed by the other monomer. The compound in FIG. 2B comprises a phenyl moiety that fits to the binding pocket formed by Phe 37, Ser 38, Cys 39, Gln 40 and Pro 58 of both monomers.

FIG. 3 shows yet another compound docked to the NS5A surface depicted in FIG. 2A. The docking pose was generated using Insight II (Accelrys, San Diego, Calif.). The carbon, nitrogen, oxygen, and sulfur atoms in the compound are depicted in cyan, blue, red, and yellow, respectively. The solvent-accessible molecular surface of the NS5A dimer is depicted in two colors, the orange area corresponding to the surface formed by one monomer and the green area corresponding the surface formed by the other monomer. The compound fits to the interaction sites formed by each monomer. In addition, the compound comprises a phenyl moiety that fits to the binding pocket formed by Phe 37, Ser 38, Cys 39, Gln 40 and Pro 58 of both monomers.

FIG. 4 illustrates the alignment of NS5A sequences of different HCV genotypes.

### DETAILED DESCRIPTION

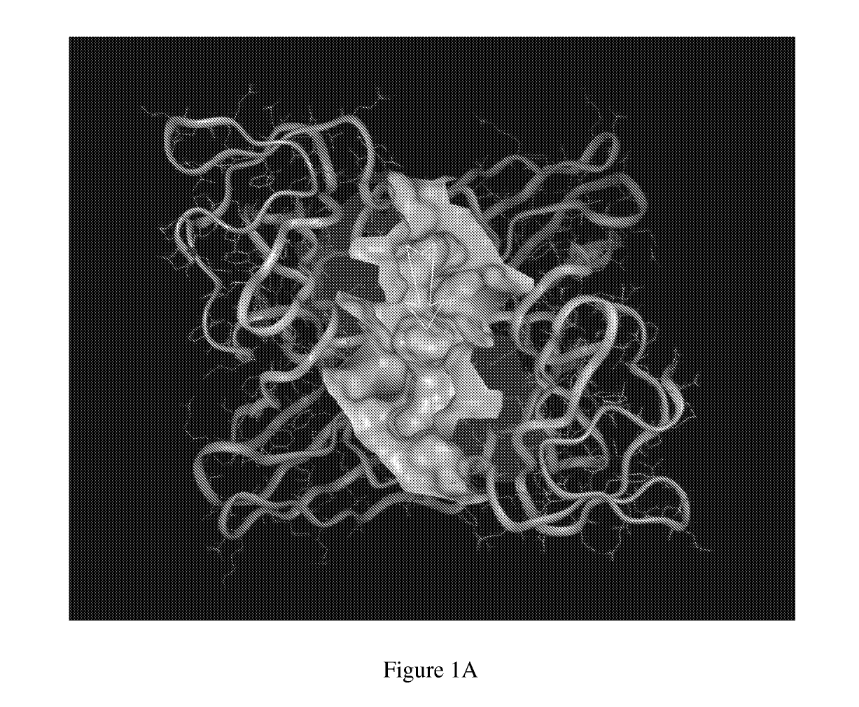

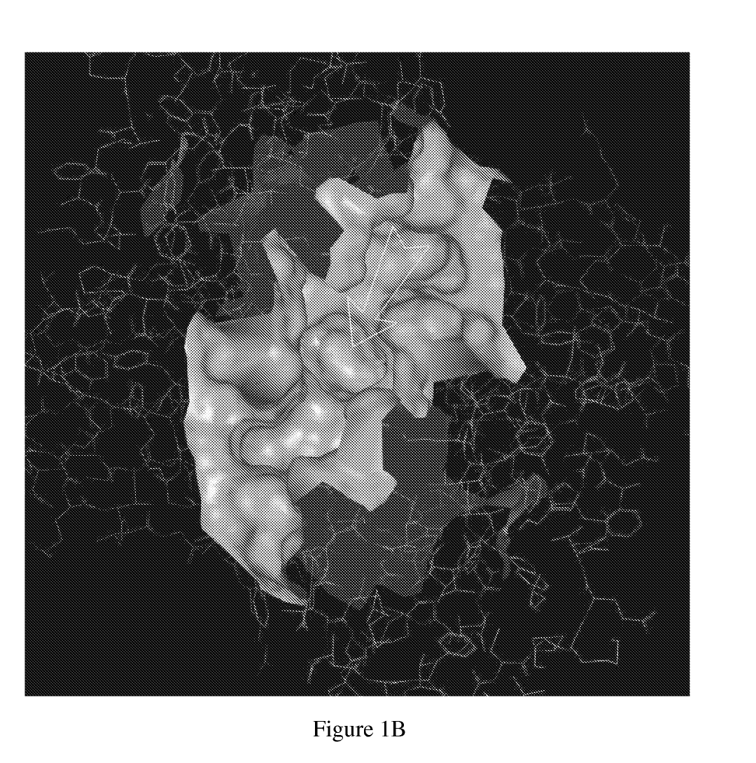

The present invention features an unexpected discovery of a unique binding site on NS5A dimers. Compounds with moieties that fit to this binding site exhibited significantly improved anti-HCV activities as compared to compounds without such moieties. The amino acid residues that form this binding site comprise Phe 37, Ser 38, Cys 39 and Pro 58 of both NS5A monomers in an NS5A dimer. Preferably, the amino acid residues that form this binding site comprise Phe 37, Ser 38, Cys 39, Gln 40 and Pro 58 of both NS5A monomers. FIGS. 1A and 1B reveal the solvent-accessible molecular surface of this binding site, as indicated by the arrow in each Figure. The surface formed by one monomer is depicted in orange, and that formed by the other monomer is depicted in green.

As used herein, the numbering of each amino acid residue in an NS5A monomer is in reference to SEQ ID NO:1 (the NS5A sequence of HCV 1b-Con1) and is determined by the position of the corresponding residue in SEQ ID NO:1 when the NS5A monomer is aligned to SEQ ID NO:1. Therefore, amino acid 37, 38, 39 and 58 of an NS5A monomer refer to the amino acid residues that correspond to amino acids 37, 38, 39 and 58 of SEQ ID NO:1, respectively, when the NS5A monomer is aligned to SEQ ID NO:1. Likewise, an NS5A monomer comprising Phe 37, Ser 38, Cys 39 and Pro 58 means that the amino acid residues in the NS5A monomer that correspond to residues 37, 38, 39 and 58 of SEQ ID NO:1 in the sequence alignment are phenylalanine, serine, cysteine, and proline, respectively. Notwithstanding the above, the numbering of each amino acid residue in an NS5A monomer that is depicted by a SED ID NO is determined by the position of the amino acid residue in the SEQ ID NO.

The present invention also features the identification of another binding site on NS5A dimers. The amino acid residues that form this binding site comprise Ser 81, His 85, Leu 110, Glu 152, Gly 155, Cys 165, and Leu 168 of one monomer in an NS5A dimer. Preferably, the amino acid residues that form this binding site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of one monomer in the NS5A dimer. Furthermore, the present invention feature the identification of yet another binding site on NS5A dimers. The amino acid residues that form this binding site comprise Ser 81, His 85, Leu 110, Glu 152, Gly 155, Cys 165, and Leu 168 of both monomers in an NS5A dimer. Preferably, the amino acid residues that form this binding site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of both monomers in the NS5A dimer. Many NS5A inhibitors fit to these binding sites in computer docking models, indicating that interactions with these binding sites can disrupt NS5A functions.

U.S. Patent Application Publication No. 20090004111 and Tellinghuisen et al., NATURE 435:374-379 (2005), describe a crystal structure of an NS5A dimer prepared from purified NS5A Domain I-Δh. NS5A Domain I-Δh comprises amino acids 25-216 of SEQ ID NO:1 as well as a C-terminal tag. The dimensions of the crystal unit cell are a,b=55.28 Å, c=312.30 Å, α,β,γ=90°, with two NS5A monomers per asymmetric unit. The final computer model of this NS5A dimer (hereinafter the “1ZH1 dimer”) contains 96 solvent molecules, two zinc atoms, and two NS5A monomers each of which consists of amino acids 36-198 of SEQ ID NO:1. The atomic coordinates of the 1ZH1 dimer have been deposited in the RCSB Protein Data Bank under accession number 1ZH1 and are incorporated herein by reference. See Tellinghuisen et al., supra. The 1ZH1 dimer has a “claw-like” shape which comprises a relatively flat, basic region formed by the N terminal portions of the two monomers and a large groove formed by the C terminal portions of the monomers. The relatively flat, basic region allows the NS5A protein to be in close contact with the negatively charged lipid head groups of the membrane, thereby positioning the large groove to face away from the membrane. The large groove is an attractive nucleic acid binding pocket, and is of sufficient dimensions to bind to either single or double stranded RNA molecules. The deep, highly basic portion of the groove has a diameter of about 13.5 angstroms (Å), and the boundary region of the groove has a diameter of about 27.5 Å. An RNA molecule can easily fit in the groove, making both electrostatic contacts with the deep basic groove and others contacts with the groove boundary region. The “arms” extending out past the groove are more acidic and may serve as a clamp to prevent RNA from exiting the groove. See Tellinghuisen et al., supra.

A molecular surface analysis of the 1ZH1 dimer showed a conserved region on the outside surface of the groove. See Tellinghuisen et al., supra. The major components of this conserved region include isoleucine 90, tryptophan 111, proline 141, gulatmine 143, proline 145, histidine 159, glycine 178, proline 192, and glutamate 193. These residues are relatively conserved among different HCV genotypes. An electronic potential plot of the region revealed a complex mixture of acidic, basic, and hydrophobic residues. See Tellinghuisen et al., supra. In addition, this conserved region includes a prominent pocket generated by the interface between the elements. It is postulated that this conserved region is involved in many interactions between NS5A and other proteins. See U.S. Patent Application Publication No. 20090004111.

Computer docking models also suggested that NS5A inhibitors may interact with the outside surface of an NS5A dimer, or interfere with its dimerization. See, e.g., Conte et al. BIOORG. MED. CHEM. LETT. 19: 1779-1783 (2009). However, these models fail to explain the structure-activity relationship of many NS5A inhibitors, and in some cases, fail to generate consistent binding poses for NS5A inhibitors.

The present invention discovered that, as opposed to binding to the outside surface of an NS5A dimer or interfering with NS5A dimerization, many NS5A inhibitors interact with amino acid residues located on the inside surface of NS5A dimers. For instance, the present invention identified a binding site comprising Ser 81, His 85, Leu 110, Glu 152, Gly 155, Cys 165, and Leu 168 of both monomers of an NS5A dimer. Preferably, the binding site comprises Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of both monomers. These amino acid residues are primarily located on the inside surface of the large, RNA-binding groove formed by the NS5A dimers. Many NS5A inhibitors fit, or can be docked, to this binding site in computer docking models.

The present invention also identified another binding site comprising Phe 37, Ser 38, Cys 39 and Pro 58 of both monomers in an NS5A dimer. Preferably, the binding site comprises Phe 37, Ser 38, Cys 39, Gln 40 and Pro 58 of both monomers. These amino acid residues are also primarily located on the inside surface of the large, RNA-binding groove. Compounds having moieties that fit to this binding site exhibit significantly improved NS5A inhibitory activities than compounds without such moieties.

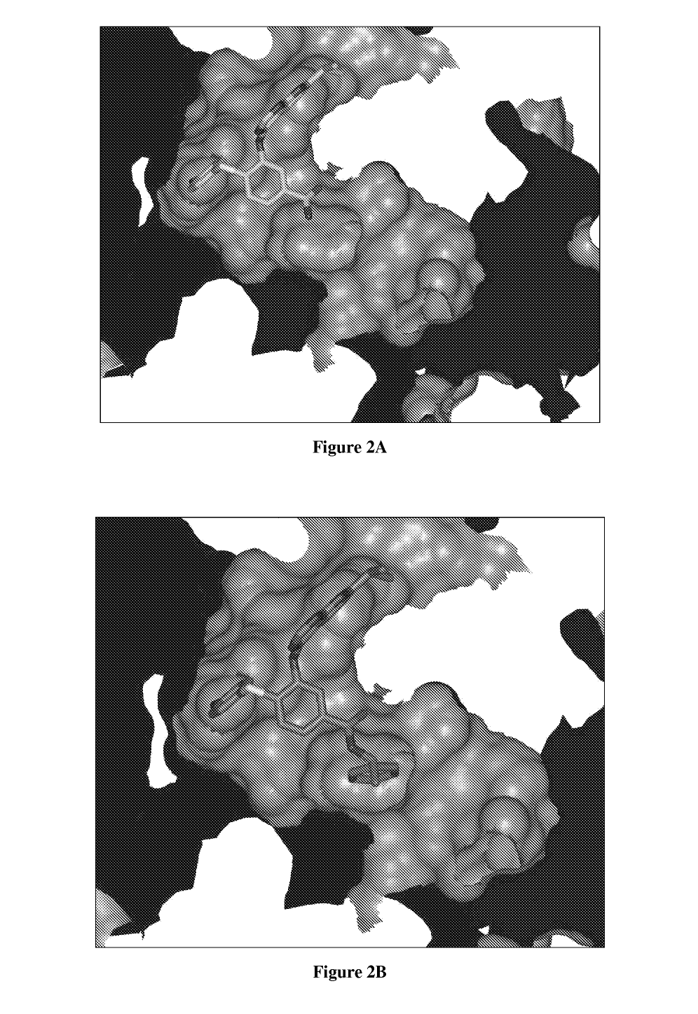

FIG. 2A shows an NS5A inhibitor

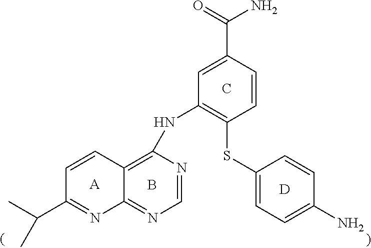

docked to the 1ZH1 dimer in a computer docking model. As shown in the model, the A and B rings in the compound fit into a cleft formed by Arg 41, Gly 42, Ser 81, Asn 82, His 85 and Thr 87 of one monomer, and the D ring fits into a hydrophobic tunnel formed by Phe 37, Ser 38, Cys 39, Ser 81, Arg 157, Ala 162, Pro 163, and Ala 164 of the same monomer. FIG. 2B illustrates another NS5A inhibitor

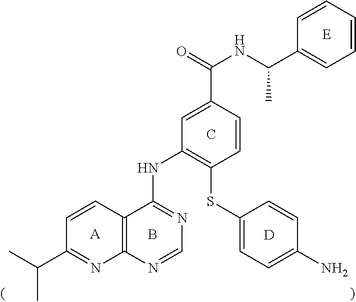

docked to the 1ZH1 dimer, where the A and B rings fit into the same cleft, and the D ring fits into the same hydrophobic tunnel, as depicted in FIG. 2A. The additional E ring in the compound of FIG. 2B fits into a binding pocket formed by Phe 37, Ser 38, Cys 39, Gln 40 and Pro 58 of both monomers of the NS5A dimer. When tested in vitro using HCV 1a replicon assays, the compound of FIG. 2B showed over 1,000-fold increase in HCV replication inhibitory activities compared to the compound of FIG. 2A. Likewise, when tested in vitro using HCV 1b replicon assays, the compound of FIG. 2B showed over 600-fold increase in HCV replication inhibitory activities compared to the compound of FIG. 2A.

Accordingly, in one aspect, the present invention features a method of identifying NS5A inhibitors. The method comprises docking a compound to an NS5A dimer using a computer docking program, wherein the NS5A dimer comprises two NS5A monomers and a binding site, and the amino acid residues that form the binding site comprise Phe 37, Ser 38, Cys 39 and Pro 58 of both said monomers. That at least a portion of the compound fits to the binding site is indicative that the compound is an NS5A inhibitor. Preferably, the amino acid residues that form the binding site comprise Phe 37, Ser 38, Cys 39, Gln 40 and Pro 58 of both monomers. More preferably, the binding site has a pocket shape, and the compound comprises a moiety that fits to the pocket.

Moieties that can fit into this binding site include, but are not limited to, -L-E, wherein:
E is C₃-C₁₄carbocycle or 3- to 14-membered heterocycle, and is optionally substituted with one or more R_{A}; or E is L_{S}-R_{E};
L is -L_{S}-, -L_{S}-O-L_{S}′-, -L_{S}-C(O)-L_{S}′-, -L_{S}-S(O)₂-L_{S}′-, -L_{S}-S(O)-L_{S}′-, -L_{S}-OS(O)₂-L_{S}′-, -L_{S}-S(O)₂O-L_{S}′-, -L_{S}-OS(O)-L_{S}′-, -L_{S}-S(O)O-L_{S}′-, -L_{S}-C(O)O-L_{S}′-, -L_{S}-OC(O)-L_{S}′-, -L_{S}-OC(O)O-L_{S}′-, -L_{S}-C(O)N(R_{B})-L_{S}′-, L_{S}-N(R_{B})C(O)-L_{S}′-, -L_{S}-C(O)N(R_{B})O-L_{S}′-, -L_{S}-N(R_{B})C(O)O-L_{S}′-, -L_{S}-OC(O)N(R_{B})-L_{S}′-, -L_{S}-C(O)N(R_{B})N(R_{B}′)-L_{S}′-, -L_{S}-S-L_{S}′-, -L_{S}-C(S)-L_{S}′-, -L_{S}-C(S)O-L_{S}′-, -L_{S}-OC(S)-L_{S}′-, -L_{S}-C(S)N(R_{B})-L_{S}′-, -L_{S}-N(R_{B})-L_{S}′-, -L_{S}-N(R_{B})C(S)-L_{S}′-, -L_{S}-N(R_{B})S(O)-L_{S}′-, -L_{S}-N(R_{B})S(O)₂-L_{S}′-, -L_{S}-S(O)₂N(R_{B})-L_{S}′-, -L_{S}-S(O)N(R_{B})-L_{S}′-, -L_{S}-C(S)N(R_{B})O-L_{S}′-, -L_{S}-C(O)N(R_{B})C(O)-L_{S}′-, -L_{S}-N(R_{B})C(O)N(R_{B}′)-L_{S}′-, -L_{S}-N(R_{B})SO₂N(R_{B}′)-L_{S}′-, -L_{S}-N(R_{B})S(O)N(R_{B}′)-L_{S}′-, or -L_{S}-C(S)N(R_{B})N(R_{B}′)-L_{S}′-;
L_{S} and L_{S}′ are each independently selected at each occurrence from bond; or C₁-C₆alkylene, C₂-C₆alkenylene or C₂-C₆alkynylene, each of which is independently optionally substituted at each occurrence with one or more R_{L};
R_{A} is independently selected at each occurrence from halogen, oxo, thioxo, hydroxy, mercapto, nitro, cyano, amino, carboxy, formyl, phosphonoxy, or phosphono; or -L_{S}-R_{E};
R_{B} and R_{B}′ are each independently selected at each occurrence from hydrogen; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆carbocycle or 3- to 6-membered heterocycle; or C₃-C₆carbocycle or 3- to 6-membered heterocycle; wherein each C₃-C₆carbocycle or 3- to 6-membered heterocycle in R_{B} or R_{B}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′) or —C(O)N(R_{S})C(O)—R_{S}′; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{L} is independently selected at each occurrence from halogen, nitro, oxo, phosphonoxy, phosphono, thioxo, cyano, —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′) or —N(R_{S})C(O)R_{S}′; or C₃-C₆carbocycle 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{S}, R_{S}′ and R_{S}″ are each independently selected at each occurrence from hydrogen; C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano or 3- to 6-membered carbocycle or heterocycle; or 3- to 6-membered carbocycle or heterocycle; wherein each 3- to 6-membered carbocycle or heterocycle in R_{S}, R_{S}′ or R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Preferably, the moiety that fits to this binding site comprises C₅-C₆carbocycle, 5- to 6-membered heterocycle, or 6- to 12-membered bicycle, each of which is optionally substituted with one or more R_{A} as defined above. Also preferably, the moiety comprises C₁-C₆alkyl, C₂-C₆alkenyl or C₂-C₆alkynyl, each of which is optionally substituted with one or more R_{L} as defined above. More preferably, the moiety comprises C₅-C₆carbocycle, 5- to 6-membered heterocycle, or 6- to 12-membered bicycles, each of which is optionally substituted with one or more substituents selected from halogen, hydroxy, mercapto, amino, carboxy, nitro, oxo, phosphonoxy, phosphono, thioxo, cyano, C₁-C₆alkyl, C₂-C₆alkenyl or C₂-C₆alkynyl, wherein each of said C₁-C₆alkyl, C₂-C₆alkenyl or C₂-C₆alkynyl can be further independently optionally substituted at each occurrence with one or more substituents selected from halogen, hydroxy, mercapto, amino, carboxy, nitro, oxo, phosphonoxy, phosphono, thioxo, formyl, cyano, C₃-C₆carbocycle or 3- to 6-membered heterocycle. Highly preferably, the moiety comprises C₅-C₆carbocycle, 5- to 6-membered heterocycle, or 6- to 12-membered bicycles, each of which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

In one example, the moiety that fits to this binding site comprises phenyl optionally substituted with one or more substituents selected from is halogen, hydroxy, mercapto, amino, carboxy, C₁-C₆alkyl, C₂-C₆alkenyl or C₂-C₆alkynyl, wherein each of said C₁-C₆alkyl, C₂-C₆alkenyl or C₂-C₆alkynyl is independently optionally substituted at each occurrence with one or more substituents selected from halogen, hydroxy, mercapto, amino or carboxy. In another example, the moiety comprises C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl or cyano.

In another aspect, a moiety that fits into the binding site identified by the present invention comprises or consists of -L₃-D, wherein:
L₃ is bond or -L_{S}-K-L_{S}′-, wherein K is selected from bond, —O—, —S—, —N(R_{B})—, —C(O)—, —S(O)₂—, —S(O)—, —OS(O)—, —OS(O)₂—, —S(O)₂O—, —S(O)O—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —C(O)N(R_{B})C(O)—, —N(R_{B})C(O)N(R_{B}′)—, —N(R_{B})SO₂N(R_{B}′)—, or —N(R_{B})S(O)N(R_{B}′)—;
D is C₃-C₁₂carbocycle or 3- to 12-membered heterocycle, and is optionally substituted with one or more R_{A}; or D is C₃-C₁₂carbocycle or 3- to 12-membered heterocycle which is substituted with J and optionally substituted with one or more R_{A}, where J is C₃-C₁₂carbocycle or 3- to 12-membered heterocycle and is optionally substituted with one or more R_{A}, or J is —SF₅; or D is hydrogen or R_{A};
R_{A} is independently selected at each occurrence from halogen, nitro, oxo, phosphonoxy, phosphono, thioxo, cyano, or -L_{S}-R_{E}, wherein two adjacent R_{A}, taken together with the atoms to which they are attached and any atoms between the atoms to which they are attached, can optionally form carbocycle or heterocycle;
R_{B} and R_{B}′ are each independently selected at each occurrence from hydrogen; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano or 3- to 6-membered carbocycle or heterocycle; or 3- to 6-membered carbocycle or heterocycle; wherein each 3- to 6-membered carbocycle or heterocycle in R_{B} or R_{B}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′ R_{S}″), —N(R_{S})S(O)N(R_{S}′ R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —P(O)(OR_{S})₂, or —C(O)N(R_{S})C(O)—R_{S}′; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S}, or —N(R_{S}R_{S}′);
R_{F} is independently selected at each occurrence from C₁-C₁₀alkyl, C₂-C₁₀alkenyl or C₂-C₁₀alkynyl, each of which contains 0, 1, 2, 3, 4 or 5 heteroatoms selected from O, S or N and is independently optionally substituted with one or more R_{L}; or —(R_{X}—R_{Y})_{Q}—(R_{X}—R_{Y}′), wherein Q is 0, 1, 2, 3 or 4, and each R_{X} is independently O, S or N(R_{B}), wherein each R_{Y} is independently C₁-C₆alkylene, C₂-C₆alkenylene or C₂-C₆alkynylene each of which is independently optionally substituted with one or more substituents selected from halogen, hydroxy, mercapto, amino, carboxy, nitro, oxo, phosphonoxy, phosphono, thioxo, formyl or cyano, and wherein each R_{Y}′ is independently C₁-C₆alkyl, C₂-C₆alkenyl or C₂-C₆alkynyl each of which is independently optionally substituted with one or more substituents selected from halogen, hydroxy, mercapto, amino, carboxy, nitro, oxo, phosphonoxy, phosphono, thioxo, formyl or cyano;
R_{L} is independently selected at each occurrence from halogen, nitro, oxo, phosphonoxy, phosphono, thioxo, cyano, —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′) or —N(R_{S})C(O)R_{S}′; or C₃-C₆carbocycle 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; wherein two adjacent R_{L}, taken together with the atoms to which they are attached and any atoms between the atoms to which they are attached, can optionally form carbocycle or heterocycle;
L_{S} and L_{S}′ are each independently selected at each occurrence from bond; or C₁-C₆alkylene, C₂-C₆alkenylene or C₂-C₆alkynylene, each of which is independently optionally substituted at each occurrence with one or more R_{L}; and
R_{S}, R_{S}′ and R_{S}″ are each independently selected at each occurrence from hydrogen; C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or 3- to 6-membered carbocycle or heterocycle; wherein each 3- to 6-membered carbocycle or heterocycle in R_{S}, R_{S}′ or R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

D preferably is selected from C₅-C₆carbocycle, 5- to 6-membered heterocycle, or 6- to 12-membered bicycles, and is optionally substituted with one or more R_{A}. D can also be preferably selected from C₁-C₆alkyl, C₂-C₆alkenyl or C₂-C₆alkynyl, and is optionally substituted with one or more substituents selected from R_{L}. More preferably, D is C₅-C₆carbocycle (e.g., phenyl), 5- to 6-membered heterocycle (e.g., pyridinyl, pyrimidinyl, thiazolyl), or 6- to 12-membered bicycles (e.g., indanyl, 4,5,6,7-tetrahydrobenzo[d]thiazolyl, benzo[d]thiazolyl, indazolyl, benzo[d][1,3]dioxol-5-yl), and is substituted with one or more R_{M}, where R_{M} is halogen, nitro, oxo, phosphonoxy, phosphono, thioxo, cyano, or -L_{S}-R_{E}. Also preferably, D is phenyl, and is optionally substituted with one or more R_{A}. More preferably, D is phenyl, and is substituted with one or more R_{M}, wherein R_{M} is as defined above. Highly preferably, D is

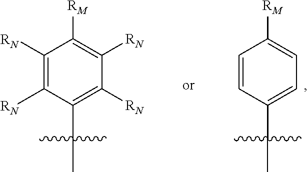

wherein R_{M} is as defined above, and each R_{N} is independently selected from R_{D} and preferably is hydrogen. One or more R_{N} can also preferably be halo such as F.

D is also preferably pyridinyl, pyrimidinyl, or thiazolyl, optionally substituted with one or more R_{A}. More preferably D is pyridinyl, pyrimidinyl, or thiazolyl, and is substituted with one or more R_{M}. Highly preferably, D is

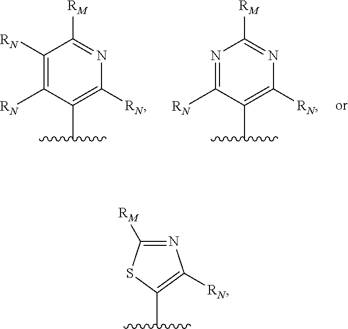

wherein R_{M} is as defined above, and each R_{N} is independently selected from R_{D} and preferably is hydrogen. One or more R_{N} can also preferably be halo such as F. D is also preferably indanyl, 4,5,6,7-tetrahydrobenzo[d]thiazolyl, benzo[d]thiazolyl, or indazolyl, and is optionally substituted with one or more R_{A}. More preferably D is indanyl, 4,5,6,7-tetrahydrobenzo[d]thiazolyl, benzo[d]thiazolyl, indazolyl, or benzo[d][1,3]dioxol-5-yl, and is substituted with one or more R_{M}. Highly preferably, D is

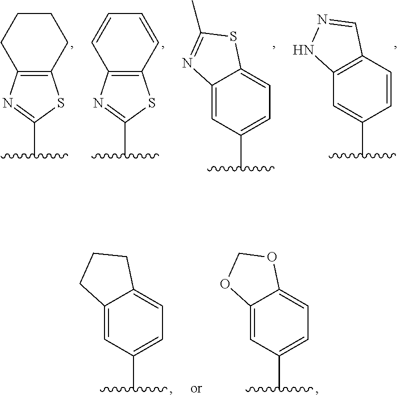

and is optionally substituted with one or more R_{M}.

Preferably, R_{M} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. More preferably, R_{M} is halogen, hydroxy, mercapto, amino, carboxy; or C₁-C₆alkyl, C₂-C₆alkenyl or C₂-C₆alkynyl, each of which is independently optionally substituted at each occurrence with one or more substituents selected from halogen, hydroxy, mercapto, amino or carboxy. Highly preferably, R_{M} is C₁-C₆alkyl which is optionally substituted with one or more substituents selected from halogen, hydroxy, mercapto, amino or carboxy.

Also preferably, R_{M} is halogen, hydroxy, mercapto, amino, carboxy, nitro, oxo, phosphonoxy, phosphono, thioxo, or cyano; or R_{M} is -L_{S}-R_{E}, wherein L_{S} is a bond or C₁-C₆alkylene, and R_{E} is —N(R_{S}R_{S}′), —O—R_{S}, —C(O)R_{S}, —C(O)OR_{S}, —C(O)N(R_{S}R_{S}′), —N(R_{S})C(O)R_{S}′, —N(R_{S})C(O)OR_{S}′, —N(R_{S})SO₂R_{S}′, —SO₂R_{S}, —SR_{S}, or —P(O)(OR_{S})₂, wherein R_{S} and R_{S}′ can be, for example, each independently selected at each occurrence from (1) hydrogen or (2) C₁-C₆alkyl optionally substituted at each occurrence with one or more halogen, hydroxy, —O—C₁-C₆alkyl or 3- to 6-membered heterocycle; or R_{M} is C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or R_{M} is C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S}, or —N(R_{S}R_{S}′). More preferably, R_{M} is halogen (e.g., fluoro, chloro, bromo, iodo), hydroxy, mercapto, amino, carboxy, or C₁-C₆alkyl (e.g., methyl, isopropyl, tert-butyl), C₂-C₆alkenyl or C₂-C₆alkynyl, each of which is independently optionally substituted at each occurrence with one or more substituents selected from halogen, hydroxy, mercapto, amino, cyano, or carboxy. For example R_{M} is CF₃, —C(CF₃)₂—OH, —C(CH₃)₂—CN, —C(CH₃)₂—CH₂OH, or —C(CH₃)₂—CH₂NH₂. Also preferably R_{M} is -L_{S}-R_{E} where L_{S} is a bond and R_{E} is —N(R_{S}R_{S}), —O—R_{S}, —N(R_{S})C(O)OR_{S}′, —N(R_{S})SO₂R_{S}′, —SO₂R_{S}, or —SR_{S}. For example where L_{S} is a bond, R_{E} is —N(C₁-C₆alkyl)₂ (e.g., —NMe₂); —N(C₁-C₆alkylene-O—C₁-C₆alkyl)₂ (e.g. —N(CH₂CH₂OMe)₂); —N(C₁-C₆alkyl)(C₁-C₆alkylene-O—C₁-C₆alkyl) (e.g. —N(CH₃)(CH₂CH₂OMe)); —O—C₁-C₆alkyl (e.g., —O-Me, —O-Et, —O-isopropyl, —O-tert-butyl, —O-n-hexyl); —O—C₁-C₆haloalkyl (e.g., —OCF₃, —OCH₂CF₃); —O—C₁-C₆alkylene-piperidine (e.g., —O—CH₂CH₂-1-piperidyl); —N(C₁-C₆alkyl)C(O)OC₁-C₆alkyl (e.g., —N(CH₃)C(O)O—CH₂CH(CH₃)₂), —N(C₁-C₆alkyl)SO₂C₁-C₆alkyl (e.g., —N(CH₃)SO₂CH₃); —SO₂C₁-C₆alkyl (e.g., —SO₂Me); —SO₂C₁-C₆haloalkyl (e.g., —SO₂CF₃); or —S—C₁-C₆haloalkyl (e.g., SCF₃). Also preferably R_{M} is -L_{S}-R_{E} where L_{S} is C₁-C₆alkylene (e.g., —CH₂—, —C(CH₃)₂—, —C(CH₃)₂—CH₂—) and R_{E} is —O—R_{S}, —C(O)OR_{S}, —N(R_{S})C(O)OR_{S}′, or —P(O)(OR_{S})₂. For example R_{M} is —C₁-C₆alkylene-O—R_{S} (e.g., —C(CH₃)₂—CH₂—OMe); —C₁-C₆alkylene-C(O)OR_{S} (e.g., —C(CH₃)₂—C(O)OMe); —C₁-C₆alkylene-N(R_{S})C(O)OR_{S}′ (e.g., —C(CH₃)₂—CH₂—NHC(O)OCH₃); or —C₁-C₆alkylene-P(O)(OR_{S})₂ (e.g., —CH₂—P(O)(OEt)₂). Also more preferably R_{M} is C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S}, or —N(R_{S}R_{S}′). For example R_{M} is cycloalkyl (e.g., cyclopropyl, 2,2-dichloro-1-methylcycloprop-1-yl, cyclohexyl), phenyl, heterocyclyl (e.g., morpholin-4-yl, 1,1-dioxidothiomorpholin-4-yl, 4-methylpiperazin-1-yl, 4-methoxycarbonylpiperazin-1-yl, pyrrolidin-1-yl, piperidin-1-yl, 4-methylpiperidin-1-yl, 3,5-dimethylpiperidin-1-yl, 4,4-difluoropiperidin-1-yl, tetrahydropyran-4-yl, pyridinyl, pyridin-3-yl, 6-(dimethylamino)pyridin-3-yl). Highly preferably, R_{M} is C₁-C₆alkyl which is optionally substituted with one or more substituents selected from halogen, hydroxy, mercapto, amino or carboxy (e.g., tert-butyl, CF₃).

More preferably, D is C₅-C₆carbocycle, 5- to 6-membered heterocycle or 6- to 12-membered bicycle and is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle, wherein said C₃-C₆carbocycle or 3- to 6-membered heterocycle is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Also preferably, D is C₅-C₆carbocycle or 5- to 6-membered heterocycle and is substituted with J and optionally substituted with one or more R_{A}, and J is C₃-C₆carbocycle or 3- to 6-membered heterocycle and is optionally substituted with one or more R_{A}, and preferably, J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Also preferably, D is C₅-C₆carbocycle or 5- to 6-membered heterocycle and is substituted with J and optionally substituted with one or more R_{A}, and J is 6- to 12-membered bicycle (e.g., a 7- to 12-membered fused, bridged or sipro bicycle comprising a nitrogen ring atom through which J is covalently attached to D) and is optionally substituted with one or more R_{A}. More preferably, D is phenyl and is substituted with J and optionally substituted with one or more R_{A}, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Highly preferably, D

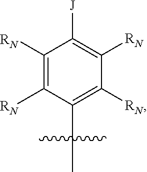

wherein each R_{N} is independently selected from R_{D} and preferably is hydrogen or halogen, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Also preferably, D is

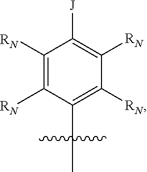

wherein each R_{N} is independently selected from R_{D} and preferably is hydrogen or halogen, and J is C₃-C₆carbocycle and 3- to 6-membered heterocycle and is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Also preferably, D is

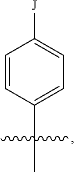

and J is C₃-C₆carbocycle or 3- to 6-membered heterocycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′).

In yet another aspect, the present invention features another method of identifying NS5A inhibitors. The method comprises docking a compound to an NS5A dimer using a computer docking program, wherein the NS5A dimer comprises two NS5A monomers and a binding site, and the amino acid residues that form the binding site comprise Ser 81, His 85, Leu 110, Glu 152, Gly 155, Cys 165, and Leu 168 of one said monomer. That at least a portion of the compound fits to the binding site is indicative that the compound is an NS5A inhibitor. Preferably, the amino acid residues that form the binding site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of one NS5A monomer. In one embodiment, a substantial portion of the compound fits to this binding site. In another embodiment, the whole compound fits to this binding site.

In yet another aspect, the present invention features yet another method of identifying NS5A inhibitors. The method comprising docking a compound to an NS5A dimer using a computer docking program, wherein the NS5A dimer comprises two NS5A monomers and a binding site, and the amino acid residues that form the binding site comprise Ser 81, His 85, Leu 110, Glu 152, Gly 155, Cys 165, and Leu 168 of one said monomer, and Phe 37, Ser 38, Cys 39 and Pro 58 of both said monomers. That at least a portion of the compound fits to the binding site is indicative that the compound is an NS5A inhibitor. Preferably, the amino acid residues that form the binding site comprise Ser 81, His 85, Leu 110, Glu 152, Gly 155, Cys 165, and Leu 168 of one monomer, and Phe 37, Ser 38, Cys 39, Gln 40 and Pro 58 of both monomers. Also preferably, the amino acid residues that form the binding site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of one monomer, and Phe 37, Ser 38, Cys 39 and Pro 58 of both monomers. More preferably, the amino acid residues that form the binding site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of one monomer, and Phe 37, Ser 38, Cys 39, Gln 40 and Pro 58 of both monomers. Non-limiting examples of NS5A inhibitors that can fit or be docked to this binding site, or contain moieties that fit to this binding site, include many compounds described in U.S. Patent Application Publication No. 20070232627, the entire content of which is incorporated herein by reference. In one embodiment, a substantial portion of the compound fits to this binding site. In another embodiment, the whole compound fits to this binding site.

In still another aspect, the present invention features still another method of identifying NS5A inhibitors. The method comprises docking a compound to an NS5A dimer using a computer docking program, wherein the NS5A dimer comprises two NS5A monomers and a binding site, and the amino acid residues that form the binding site comprise Ser 81, His 85, Leu 110, Glu 152, Gly 155, Cys 165, and Leu 168 of both said monomers. That at least a portion of the compound fits to the binding site is indicative that the compound is an NS5A inhibitor. Preferably, the amino acid residues that form the binding site comprise Ser 81, His 85, Leu 110, Glu 152, Gly 155, Cys 165, and Leu 168 of one monomer, and Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of the other monomer. More preferably, the amino acid residues that form the binding site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of both monomers. Because the binding site includes amino acid residues from both monomers, a compound identified according to this aspect of the invention often has a dimeric or dimeric-like structure, with one part of the compound fitting to the interaction site provided by one monomer and the other part fitting to the interaction site provided by the other monomer. The two parts of the compound can be the same or different. In one embodiment, the compound has a symmetrical structure and consists of two identical moieties, with one moiety fitting to the interaction site provided by one monomer and the other moiety fitting to the interaction site provided by the other monomer. Non-limiting examples of NS5A inhibitors that can fit or be docked to this binding site, or contain moieties that fit to this binding site, include those described in U.S. Patent Application Ser. Nos. 61/140,318 (David A. DeGoey et al. entitled “Anti-viral Compounds” and filed Dec. 23, 2008) and 61/140,262 (David A. DeGoey et al. entitled “Anti-viral Compounds” and filed Dec. 23, 2008) and U.S. Patent Application Publication Nos. 20080299075, 20090068140 and 20090202478, all of which are incorporated herein by reference in their entireties. In one embodiment, a substantial portion of the compound of interest fits to this binding site. In another embodiment, the whole compound fits or is docked to this binding site.

In still yet another aspect, the present invention features still yet another method of identifying NS5A inhibitors. The method comprises docking a compound to an NS5A dimer using a computer docking program, wherein the NS5A dimer comprises two NS5A monomers and a binding site, and the amino acid residues that form the binding site comprise Phe 37, Ser 38, Cys 39, Pro 58, Ser 81, His 85, Leu 110, Glu 152, Gly 155, Cys 165, and Leu 168 of both said monomers. That at least a portion of the compound fits to the binding site is indicative that the compound is an NS5A inhibitor. Preferably, the amino acid residues that form the binding site comprise Phe 37, Ser 38, Cys 39, Gln 40, Pro 58, Ser 81, His 85, Leu 110, Glu 152, Gly 155, Cys 165, and Leu 168 of both monomers. More preferably, the amino acid residues that form the binding site comprise Phe 37, Ser 38, Cys 39, Arg 41, Gly 42, Pro 58, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of both monomers. Highly preferably, the amino acid residues that form the binding site comprise Phe 37, Ser 38, Cys 39, Gln 40, Arg 41, Gly 42, Pro 58, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of both monomers. Because the binding site includes amino acid residues from both monomers, a compound identified according to this aspect of the invention often has a dimeric or dimeric-like structure and comprises at least three moieties. The first moiety fits to the interaction site formed by Ser 81, His 85, Leu 110, Glu 152, Gly 155, Cys 165, and Leu 168 (or preferably, by Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168) of one monomer, the second moiety fits to the interaction site formed by Ser 81, His 85, Leu 110, Glu 152, Gly 155, Cys 165, and Leu 168 (or preferably, by Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168) of the other monomer, and the third moiety fits to the region formed by Phe 37, Ser 38, Cys 39 and Pro 58 (or preferably, by Phe 37, Ser 38, Cys 39, Gln 40 and Pro 58) of both monomers. The first and second moieties can be the same or different. Non-limiting examples of the third moiety include -L-E, as defined hereinabove. In one embodiment, a substantial portion of the compound of interest fits to this binding site. In another embodiment, the whole compound fits or is docked to this binding site.

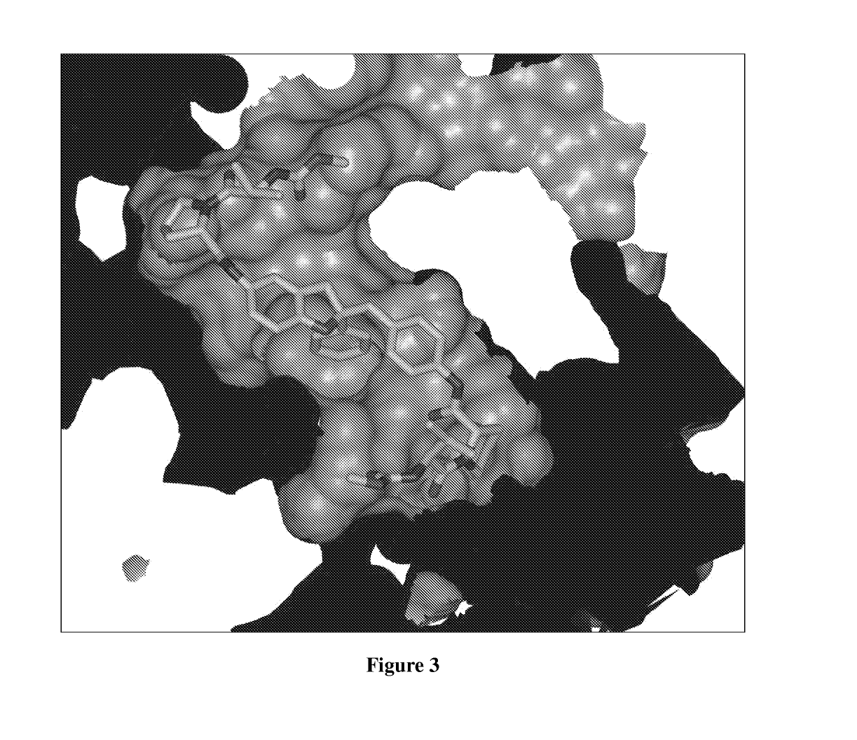

FIG. 3 shows a compound

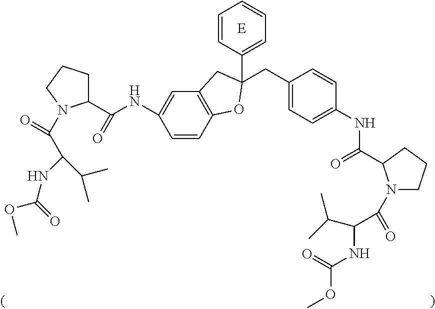

docked to the binding site described immediately above. The compound contains three moieties, where the left moiety fits to the interaction site comprising Arg 41, Gly 42, Ser 81, Asn 82, His 85, Thr 87, Leu 110, Tyr 118, Thr 151, Glu 152, Gly 155, Arg 157, Ala 162, Pro 163, Ala 164, Cys 165, Lys 166, and Leu 168 of one monomer (in orange), the right moiety fits to the interaction site comprising Arg 41, Gly 42, Ser 81, Asn 82, His 85, Thr 87, Leu 110, Tyr 118, Thr 151, Glu 152, Gly 155, Arg 157, Ala 162, Pro 163, Ala 164, Cys 165, Lys 166, and Leu 168 of the other monomer (in green), and the third moiety (the E ring) fits to the interaction site comprising Phe 37, Ser 38, Cys 39, Gln 40 and Pro 58 of both monomers.

Computer programs that are suitable for docking a compound to an NS5A dimer are well known in the art. Non-limiting examples include those described in U.S. Pat. No. 7,065,453; Yu et al., CHEM. BIOL. DRUG DES. 69:204-211 (2007); Rao et al., J. CHEM. INF. MODEL. 47:2159-2171 (2007); Sato et al., J. CHEM. INF. MODEL. 46:2552-2662 (2006); Warren et al., J. MED. CHEM. 49:5912-5931 (2006); Sousa et al., PROTEINS 65:15-26 (2006); Gastreich et al., JOURNAL OF COMPUTER-AIDED MOLECULAR DESIGN 20:717-734 (2006); Halperin et al., PROTEINS 47:409-443 (2002); and Jones et al. CURRENT OPINION IN BIOTECHNOLOGY 6:652-656 (1995), all of which are incorporated herein by reference in their entireties. Preferred docking programs include, but are not limited to, Insight II (Accelrys Software Inc., San Diego, Calif.), FlexX (Rarey et al. J. MOL. BIOL. 261:470-489 (1996), available from BioSolveIT GmbH, Germany); GRAMM; GRID (Goodford, J. MED. CHEM. 28:849-857 (1985) and Hubbard, NATURE STRUCT. BIOL. 6:711-4 (1999), available from Molecular Discovery, Perugia, IT), MCSS (Miranker and Karplus, PROTEINS: STRUCTURE, FUNCTION AND GENETICS 11: 29-34 (1991), available from Accelrys, San Diego, Calif.), AUTODOCK (Goodsell and Olsen, PROTEINS: STRUCTURE. FUNCTION, AND GENETICS 8:195-202 (1990), available from Scripps Research Institute, La Jolla, Calif.), DOCK (Kuntz et al. J. MOL. BIOL. 161:269-288 (1982), available from University of California, San Francisco, Calif.), GOLD (Jones et al. J. MOL. BIOL., 245:43-53 (1995), and Jones et al. J. MOL. BIOL., 267:727-748 (1997)), and ICM (Abagyan et al. J. COMPUT. CHEM. 15:488-506 (1994)).

These docking programs have been widely used to study protein-ligand interactions. For instance, GRAMM is a publically available program for protein docking. To predict the structure of a protein-ligand complex, the program requires only the atomic coordinates of the two molecules (e.g., an NS5A dimer and a compound of interest), and no information about the binding sites is needed. The program performs an exhaustive 6-dimensional search through the relative translations and rotations of the molecules. The GRAMM methodology is an empirical approach to smoothing the intermolecular energy function by changing the range of the atom-atom potentials. The technique locates the area of the global minimum of intermolecular energy for structures of different accuracy. The quality of the prediction depends on the accuracy of the structures. AUTODOCK is designed to predict how small molecules, such as drug candidates, bind to a receptor of known 3D structure. FlexX predicts protein-ligand interactions. For a protein with known three-dimensional structure (e.g., an NS5A dimer) and a compound of interest, FlexX predicts the geometry of the protein-ligand complex and estimates the binding affinity.

Docking a compound often includes a process of selecting such position of the compound in the interaction site of the target protein (e.g., an NS5A dimer), in which the compound has the best score. Therefore, the success of a docking program often depends on two components: the search algorithm and the scoring function. Many docking programs account for a flexible ligand, and several are attempting to model a flexible protein receptor. Each “snapshot” of the pair is referred to as a pose. There are many strategies for sampling the search space, such as using a coarse-grained molecular dynamics simulation to propose energetically reasonable poses, or using a “linear combination” of multiple structures determined for the same protein to emulate receptor flexibility, or using a genetic algorithm to “evolve” new poses that are successively more and more likely to represent favorable binding interactions. The scoring function takes a pose as input and returns a number indicating the likelihood that the pose represents a favorable binding interaction. The scores or approaches to predicting the ligand-protein interaction from the structure and position of the protein and the ligand may be divided into several groups, such as molecular dynamics, physical methods based on force fields, empirical and knowledge based. See Gohlke and Klebe, ANGEW. CHEM. INT. ED. 41:2644-2676 (2002). Many scoring functions are physics-based molecular mechanics force fields that estimate the energy of the pose; a low (negative) energy indicates a stable system and thus a likely binding interaction. An alternative approach is to derive a statistical potential for interactions from a large database of protein-ligand complexes, such as the Protein Data Bank, and evaluate the fit of the pose according to this inferred potential. To reduce the number of false positives, the energy of the top scoring poses can be recalculated using computationally more intensive techniques such as Generalized Born or Poisson-Boltzmann methods. See Feig et al., JOURNAL OF COMPUTATIONAL CHEMISTRY 25:265-84 (2004). Correct score is often expected to be proportional to the binding affinity or the binding free energy of the protein-compound interaction.

Docking can also be accomplished by using software such as QUANTA and SYBYL, followed by energy minimization and molecular dynamics with standard molecular mechanics force fields, such as CHARMM and AMBER, as appreciated by those skilled in the art.

As used herein, a moiety of a compound fits to a binding site on an NS5A dimer if the moiety interacts with at least one amino acid residue in the binding site when the compound is docked to the NS5A dimer. Such interaction helps stabilize the compound in the docked state, and can be, without limitation, ionic interaction, hydrogen bond interaction, dipole-dipole interaction, van der Waals interaction, or any combination thereof. Preferably, the moiety interacts with at least 50% of the amino acid residues that form the binding site. More preferably, the moiety interacts with at least 75% amino acid residues that form the binding site. Highly preferably, the moiety interacts with at least 90% amino acid residues that form the binding site. Most preferably, the moiety interacts with all amino acid residues in the binding site.

The stabilization effect of “fitting” can be calculated using numerous methods known in the art. For instance, ionic or steric interactions can be modeled computationally, e.g., via a force field such as Amber (Cornell et al., JOURNAL OF THE AMERICAN CHEMICAL SOCIETY 117:5179-5197 (1995)) which may assign partial charges to atoms on the protein and compound and evaluate the electrostatic interaction energy between the protein and compound atoms using the Coulomb potential. The Amber force field may also assign van der Waals energy terms to assess the attractive and repulsive steric interactions between two atoms. Lipophilic interactions can also be modeled using a variety of means. For example, the ChemScore function (Eldridge et al., JOURNAL OF COMPUTER-AIDED MOLECULAR DESIGN 11:425-445 (1997)) assigns protein and compound atoms as hydrophobic or polar, and a favorable energy term is specified for the interaction between two hydrophobic atoms. Other methods of assessing the hydrophobic contributions to compound binding are available and well-known to one skilled in the art. Other methods of assessing interactions are also available and well-known to one skilled in the art of designing molecules.

Any docking program described herein can be used in a method of the present invention, or to determine if a compound of interest can be docked to an NS5A dimer. Preferred programs include, but are not limited to, Insight II, FlexX, GRAMM, GRID, MCSS, AUTODOCK, DOCK, GOLD, and ICM. More preferably, Insight II is used.

Preferably, the NS5A dimer used in a computer docking program is the 1ZH1 dimer. The 1ZH1 dimer comprises two NS5A monomers, each of which consists of amino acids 36-198 of SEQ ID NO:1. SEQ ID NO:1 depicts the NS5A sequence of HCV 1b-Con1.

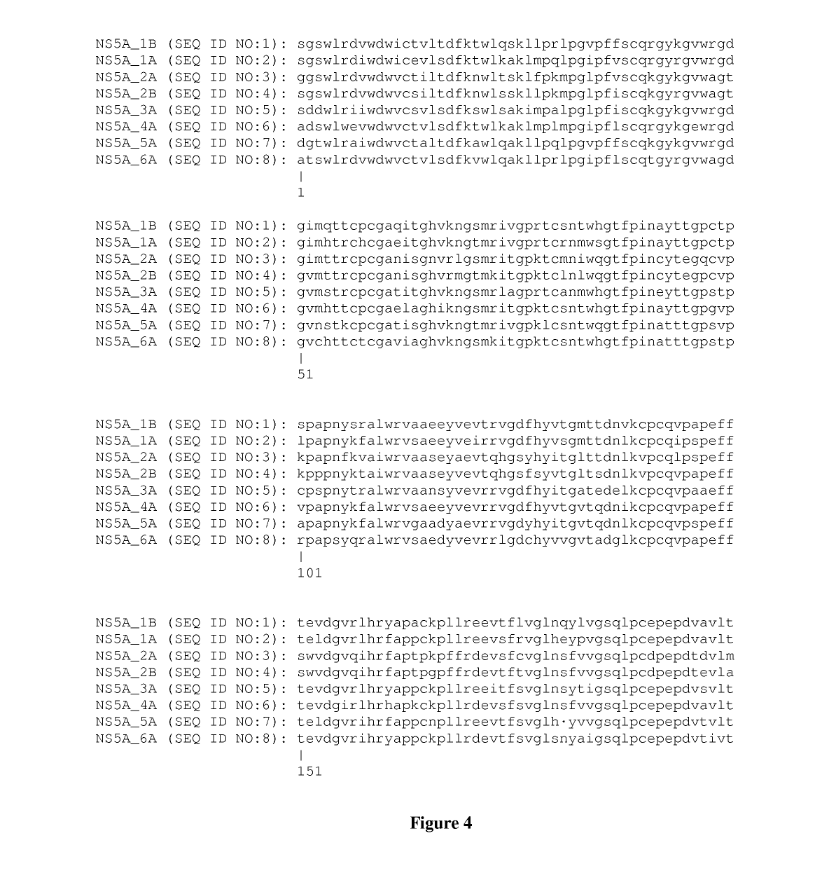

The present invention also contemplates the use of NS5A dimers of other HCV genotypes or subgenotypes. NS5A sequences of other HCV genotypes or subgenotypes are well known in the art. For example, SEQ ID NOs: 2, 3, 4, 5, 6, 7 and 8 depict NS5A sequences of HCV 1a-H77, HCV 2a-J6, HCV 2b-AAF59945, HCV 3a-Con3A, HCV 4a-DQ418782, HCV 5a-Y13184 and HCV 6a-DQ480512, respectively. FIG. 4 aligns these sequences to SEQ ID NO:1. NS5A sequences of other HCV genotypes, subgenotypes or strains can also be used, such as NS5A sequences of HCV 1 (e.g., 1a, 1b, or 1c), HCV 2 (e.g., 2a, 2b, or 2c), HCV 3 (e.g., 3a or 3b), HCV 4 (e.g., 4a, 4b, 4c, 4d, or 4e), HCV 5 (e.g., 5a), HCV 6 (e.g., 6a), HCV 7 (e.g., 7a or 7b), HCV 8 (e.g., 8a or 8b), HCV 9 (e.g., 9a), HCV 10 (e.g., 10a), and HCV 11 (e.g., 11a).

In addition, the present invention completes the use of NS5A dimers having NS5A consensus sequences. HCV consensus sequences can be created by comparing sequences of different HCV genomes. See Kolykhalov et al. SCIENCE 277:570-574 (1997), and Yanagi et al. Proc NATL ACAD SCI USA 94:8738-8743 (1997). NS5A dimers constructed based on HCV consensus sequences can be used to identify compounds that fit to the interaction sites identified by the present invention.

Preferably, each NS5A monomer in an NS5A dimer employed in the present invention has at least 50% sequence identity to amino acid 36-198 of SEQ ID NO:1. More preferably, each NS5A monomer employed in the present invention has at least 75% sequence identity to amino acid 36-198 of SEQ ID NO:1. Highly preferably, each NS5A monomer employed in the present invention has at least 90% sequence identity to amino acid 36-198 of SEQ ID NO:1. Mostly preferably, each NS5A monomer employed in the present invention has at least 95% sequence identity to amino acid 36-198 of SEQ ID NO:1.

Also preferably, each NS5A monomer employed in the present invention has at least 50% sequence identity to amino acid 37-168 of SEQ ID NO:1. More preferably, each NS5A monomer employed in the present invention has at least 75% sequence identity to amino acid 37-168 of SEQ ID NO:1. Highly preferably, each NS5A monomer employed in the present invention has at least 90% sequence identity to amino acid 37-168 of SEQ ID NO:1. Mostly preferably, each NS5A monomer employed in the present invention has at least 95% sequence identity to amino acid 37-168 of SEQ ID NO:1.

Sequence alignment and sequence identity can be determined by using, for example, the protein-protein BLAST program (i.e., BLASTP) provided by the National Center for Biotechnology Information. See also Altschul et al., J. MOL. BIOL. 215:403-410 (1990), and Altschul et al., NUCLEIC ACIDS RES. 25:3389-3402 (1997), both of which are incorporated herein by reference. BLASTP is designed to find local regions of similarity. When sequence similarity spans the whole sequence, BLASTP will also report a global alignment. The BLASTP program uses a heuristic algorithm. Preferably, the default values are used in the BLASTP program, e.g., word size is 3, scoring matrix is BLOSUM62, expect value is 10, gap existence penalty (also known as gap open penalty) is 11, and gap extension penalty is 1. Other sequence alignment methods or computer programs can also be used, as appreciated by those skilled in the art. One of ordinary skill in the art can determine appropriate parameters for measuring sequence alignment, including any algorithms needed to achieve maximal alignment over the full length of the sequences being compared. Non-limiting examples of suitable computer programs include FASTA, ClustalW, T-coffee, GGSEARCH, GLSEARCH, Dotlet, JAligner, Needle, Ngila, or SSEARCH, as well as other programs based on the dot-matrix methods, the Needleman-Wunsch algorithm, the Smith-Waterman algorithm or the word methods.

Alignment between two polypeptide sequences can also be determined manually, such as by a manual calculation of the BLASTP algorithm using the same alignment parameters as described above (e.g., score matrix is BLOSUM62, gap open penalty is 11, and gap extension penalty is 1). To begin, the two sequences are initially aligned by visual inspection. An initial alignment score is then calculated as follows: for each individual position of the alignment (i.e., for each pair of aligned residues), a numerical value is assigned according to the BLOSUM62 matrix. The sum of the values assigned to each pair of residues in the alignment is the initial alignment score. If the two sequences being aligned are highly similar, often this initial alignment provides the highest possible alignment score. The alignment with the highest possible alignment score is the optimal alignment based on the alignment parameters employed. In some instances, a higher alignment score may be obtained by introducing one or more gaps into the alignment. Whenever a gap is introduced into an alignment, a gap open penalty is assigned, and in addition a gap extension penalty is assessed for each residue position within that gap. Therefore, using the alignment parameters described above (including gap open penalty=11 and gap extension penalty=1), a gap of one residue in the alignment would correspond to a value of −(11+(1×1))=−12 assigned to the gap; a gap of three residues would correspond to a value of −(11+(3×1))=−14 assigned to the gap, and so on. This calculation can be repeated for each new gap introduced into the alignment.

Sequence identity refers to the percentage of amino acid residues in a target sequence that are identical to the amino acid residues in the reference sequence, after aligning the two sequences allowing introduction of gaps, if necessary, to achieve the maximum percent sequence identity, and not considering any conservative substitutions as part of the sequence identity.

Based on the structure of the 1ZH1 dimer, computer models for other NS5A dimers can be readily prepared using homology modeling or other methods known in the art. Homology modeling involves the construction of an atomic-resolution model of a target protein (e.g., an NS5A dimer formed by two NS5A polypeptides which have sequences homologous to amino acid 36-198 of SEQ ID NO:1) based on the three-dimensional structure of a homologous template protein (e.g., the 1ZH1 dimer) and the sequence alignment between the target protein and the template protein. This method is based on the observation that protein tertiary structure generally is better conserved than amino acid sequence. Thus, even proteins that have diverged appreciably in sequence but still share detectable similarity will also share common structural properties. In addition, similarity in amino acid sequences usually indicates significant structural similarity. As a result, the sequence alignment between the target protein (e.g., an NS5A dimer of interest) and the template protein (e.g., the 1ZH1 dimer), together with the known three-dimensional structure of the template protein, can be used to produce a structural model of the target protein. Homology modeling can produce high-quality structural models when the target and template have significant sequence similarity. Structural incoherences, e.g., structural fragments around insertions and deletions, can be modeled by screening a structural library for peptides of the desired length and with a suitable conformation. For prediction of the side chain conformation, a side chain rotamer library may be employed.

The homology modeling procedure often includes four sequential steps: template selection, target-template alignment, model construction, and model assessment. See Marti-Renom et al., ANNU REV BIOPHYS BIOMOL STRUCT 29:291-325 (2000). According to the present invention, the preferred template is the 1ZH1 dimer, and the sequence alignment preferably is carried out using the BLASTP program as described above. Given the template and the alignment, a three-dimensional structural model of the target can be generated, which is often represented as a set of Cartesian coordinates for each atom in the protein. At least three major classes of model generation methods are available: fragment assembly, segment matching, and satisfaction of spatial restraints. See Baker and Sali, SCIENCE 294:93-96 (2001). Computer program suitable for model generation include, but are not limited to, MODELLER (available from Accelrys, San Diego, Calif.). See also Eswar et al., CURRENT PROTOCOLS IN BIOINFORMATICS (John Wiley & Sons, Inc.) Supplement 15: 5.6.1-5.6.30 (2006), and Marti-Renom et al., ANNU. REV. BIOPHYS. BIOMOL. STRUCT. 29:291-325 (2000). MODELLER implements comparative protein structure modeling by satisfaction of spatial restraints, and can also perform other additional tasks, such as de novo modeling of loops in protein structures and optimization of various models of protein structure with respect to a flexibly defined objective function. See Sali and Blundell, J. MOL. BIOL. 234:779-815 (1993), and Fiser et al., PROTEIN SCIENCE 9:1753-1773 (2000). It is believed that with at least 50% sequence identity, the structures generated by homology modeling tend to be reliable and, at high sequence identities, the primary source of error in homology modeling often derives from the template on which the model is based. The accuracy of homology models may be further improved, for example, by subjecting them to molecular dynamics simulation in an effort to improve their root-mean-square deviation (RMSD) to the experimental structure.

The molecular replacement method can also be used to generate the three-dimensional structure of a target protein (e.g., an NS5A dimer formed by two NS5A polypeptides which have sequences different from amino acid 36-198 of SEQ ID NO:1) based on the known structure of the 1ZH1 dimer. In the molecular replacement method, phases can be calculated from the model and combined with the observed amplitudes to give an approximate Fourier synthesis of the structure whose coordinates are unknown. This, in turn, can be subject to any of the several forms of refinement to provide a final, accurate structure of the unknown crystal. See Lattman, METHODS IN ENZYMOLOGY, 115:55-77 (1985); Rossmann, INT. SCI. REV. SER., No. 13 (Gordon & Breach, New York, 1972). Using the structure coordinates of the 1ZH1 dimer, molecular replacement can be used to determine the structure coordinates of another NS5A dimer formed by different NS5A monomers. Non-limiting examples of computer programs suitable for molecular replacement include CNX (Brunger et al., CURRENT OPINION IN STRUCTURAL BIOLOGY 8:606-611 (1998, commercially available from Accelrys, San Diego, Calif.), MOLREP (Vagin and Teplyakov, J. APPL. CRYST. 30:1022-1025 (1997), part of the CCP4 suite), and AMoRe (Navaza, ACTA CRYST. A50:157-163 (1994)).

The two NS5A monomers in an NS5A dimer employed in the present invention can have the same or different amino acid sequences. In one embodiment, each of the two NS5A monomers comprises amino acids 37-168. As used herein, the numbering of each amino acid residue in an NS5A monomer is based on the sequence alignment between the monomer and SEQ ID NO:1. In another embodiment, each of the two NS5A monomers comprises amino acids 37-168 of SEQ ID NO: 1, 2, 3, 4, 5, 6, 7, or 8. In yet another embodiment, each of the two NS5A monomers comprises amino acids 37-168 of an NS5A polypeptide of HCV 1, 2, 3, 4, 5, 6, 7, 8, 9, 10, or 11, such as HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a. In still another embodiment, the two NS5A monomers are identical and each comprises amino acids 37-168. In a further embodiment, the two NS5A monomers are identical and each comprises amino acids 37-168 of SEQ ID NO: 1, 2, 3, 4, 5, 6, 7, or 8. In yet another embodiment, the two NS5A monomers are identical and each comprises amino acids 37-168 of an NS5A polypeptide of HCV 1, 2, 3, 4, 5, 6, 7, 8, 9, 10, or 11, such as HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a.

In still another embodiment, each of the two NS5A monomers in an NS5A dimer employed in the present invention comprises amino acids 36-198. In another embodiment, each of the two NS5A monomers comprises amino acids 36-198 of an NS5A polypeptide of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In yet another embodiment, each of the two NS5A monomers comprises amino acids 36-198 of SEQ ID NO: 1, 2, 3, 4, 5, 6 or 8 or amino acids 36-197 of SEQ ID NO:7. In a further embodiment, the two NS5A monomers are identical and each comprises amino acids 36-198. In another embodiment, the two NS5A monomers are identical and each comprises amino acids 36-198 of an NS5A polypeptide of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In yet another embodiment, the two NS5A monomers are identical and each comprises amino acids 36-198 of SEQ ID NO: 1, 2, 3, 4, 5, 6 or 8 or amino acids 36-197 of SEQ ID NO:7.

In another embodiment, each of the two NS5A monomers in an NS5A dimer employed in the present invention consists of amino acids 36-198. In one embodiment, each of the two NS5A monomers consists of amino acids 36-198 of SEQ ID NO: 1, 2, 3, 4, 5, 6 or 8 or amino acids 36-197 of SEQ ID NO:7. In still another embodiment, each of the two NS5A monomers consists of amino acids 36-198 of an NS5A polypeptide of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In yet another embodiment, both monomers are identical and each consists of amino acids 36-198. In another embodiment, both monomers are identical and each consists of amino acids 36-198 of SEQ ID NO: 1, 2, 3, 4, 5, 6 or 8 or amino acids 36-197 of SEQ ID NO:7. In still another embodiment, both monomers are identical and each consists of amino acids 36-198 of an NS5A polypeptide of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a).

In another aspect, the present invention features a method of identifying NS5A inhibitors. The method comprises docking a compound to an NS5A dimer using a computer docking program, wherein the NS5A dimer comprises two NS5A monomers and an interaction area, and the amino acid residues that form the interaction area comprise amino acids 37, 38, 39 and 58 of both NS5A monomers. That at least a portion of the compound fits to the interaction area is indicative that the compound is an NS5A inhibitor. Preferably, the amino acid residues that form the interaction area comprise amino acids 37, 38, 39, 40 and 58 of both NS5A monomers. More preferably, the interaction area has a pocket shape, and the compound comprises a moiety that fits to the pocket.

In one embodiment of this aspect of the invention, the amino acid resides that form the interaction area comprise amino acids 37, 38, 39 and 58 of both NS5A monomers, and each monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In another embodiment, the amino acid resides that form the interaction area comprise amino acids 37, 38, 39, 40 and 58 of both NS5A monomers, and each monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In still another embodiment, the amino acid resides that form the interaction area comprise amino acids 37, 38, 39 and 58 of both NS5A monomers, and each monomer comprises amino acids 37-168 of SEQ ID NO: 1, 2, 3, 4, 5, 6, 7 or 8. Preferably, each monomer comprises amino acids 36-198 of SEQ ID NO: 1, 2, 3, 4, 5, 6 or 8 or amino acids 36-197 of SEQ ID NO:7. In a further embodiment, the amino acid resides that form the interaction area comprise amino acids 37, 38, 39, 40 and 58 of both NS5A monomers, and each monomer comprises amino acids 37-168 of SEQ ID NO: 1, 2, 3, 4, 5, 6, 7 or 8. Preferably, each monomer comprises amino acids 36-198 of SEQ ID NO: 1, 2, 3, 4, 5, 6 or 8 or amino acids 36-197 of SEQ ID NO:7. In the above embodiments, each monomer preferably consists of amino acids 37-168 of SEQ ID NO: 1, 2, 3, 4, 5, 6, 7 or 8, or amino acids 36-198 of SEQ ID NO: 1, 2, 3, 4, 5, 6 or 8 or amino acids 36-197 of SEQ ID NO:7, and the two NS5A monomers preferably have the same amino acid sequence.

In yet another aspect, the present invention features a method of identifying NS5A inhibitors. The method comprises docking a compound to an NS5A dimer using a computer docking program, wherein the NS5A dimer comprises two NS5A monomers and an interaction area, and the amino acid residues that form the interaction area comprise amino acids 81, 85, 110, 152, 155, 165, and 168 of one said monomer. That at least a portion of the compound fits to the interaction area is indicative that the compound is an NS5A inhibitor. Preferably, the amino acid residues that form the interaction area comprise amino acids 41, 42, 81, 82, 85, 87, 110, 118, 151, 152, 155, 156, 157, 162, 163, 164, 165, 166, and 168 of one NS5A monomer. In one embodiment, a substantial portion of the compound fits to this interaction area. In another embodiment, the whole compound fits to this interaction area.

In one embodiment of this aspect of the invention, the amino acid resides that form the interaction area comprise amino acids 81, 85, 110, 152, 155, 165, and 168 of one NS5A monomer which comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In another embodiment, the amino acid resides that form the interaction area comprise amino acids 41, 42, 81, 82, 85, 87, 110, 118, 151, 152, 155, 156, 157, 162, 163, 164, 165, 166, and 168 of one NS5A monomer which comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In still another embodiment, the amino acid resides that form the interaction area comprise amino acids 81, 85, 110, 152, 155, 165, and 168 of one NS5A monomer which comprises amino acids 37-168 of SEQ ID NO: 1, 2, 3, 4, 5, 6, 7 or 8. Preferably, the NS5A monomer comprises amino acids 36-198 of SEQ ID NO: 1, 2, 3, 4, 5, 6 or 8 or amino acids 36-197 of SEQ ID NO:7. In a further embodiment, the amino acid resides that form the interaction area comprise amino acids 41, 42, 81, 82, 85, 87, 110, 118, 151, 152, 155, 156, 157, 162, 163, 164, 165, 166, and 168 of one NS5A monomer which comprises amino acids 37-168 of SEQ ID NO: 1, 2, 3, 4, 5, 6, 7 or 8. Preferably, the NS5A monomer comprises amino acids 36-198 of SEQ ID NO: 1, 2, 3, 4, 5, 6 or 8 or amino acids 36-197 of SEQ ID NO:7. In the above embodiments, each of the two monomers in the NS5A dimer preferably consists of amino acids 37-168 of SEQ ID NO: 1, 2, 3, 4, 5, 6, 7 or 8, or amino acids 36-198 of SEQ ID NO: 1, 2, 3, 4, 5, 6 or 8 or amino acids 36-197 of SEQ ID NO:7, and the two monomers preferably have the same amino acid sequence.

In still another aspect, the present invention features yet another method of identifying NS5A inhibitors. The method comprising docking a compound to an NS5A dimer using a computer docking program, wherein the NS5A dimer comprises two NS5A monomers and an interaction area, and the amino acid residues that form the interaction area comprise amino acids 81, 85, 110, 152, 155, 165, and 168 of one said monomer, and amino acids 37, 38, 39 and 58 of both said monomers. That at least a portion of the compound fits to the interaction area is indicative that the compound is an NS5A inhibitor. Preferably, the amino acid residues that form the interaction area comprise amino acids 81, 85, 110, 152, 155, 165, and 168 of one monomer, and amino acids 37, 38, 39, 40 and 58 of both monomers. Also preferably, the amino acid residues that form the interaction area comprise amino acids 41, 42, 81, 82, 85, 87, 110, 118, 151, 152, 155, 156, 157, 162, 163, 164, 165, 166, and 168 of one monomer, and amino acids 37, 38, 39 and 58 of both monomers. More preferably, the amino acid residues that form the interaction area comprise amino acids 41, 42, 81, 82, 85, 87, 110, 118, 151, 152, 155, 156, 157, 162, 163, 164, 165, 166, and 168 of one monomer, and amino acids 37, 38, 39, 40 and 58 of both monomers. In one embodiment, a substantial portion of the compound fits to this interaction area. In another embodiment, the whole compound fits to this interaction area.

In one embodiment of this aspect of the invention, the amino acid resides that form the interaction area comprise amino acids 81, 85, 110, 152, 155, 165, and 168 of one NS5A monomer, and amino acids 37, 38, 39 and 58 of both NS5A monomers, wherein each NS5A monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In another embodiment, the amino acid resides that form the interaction area comprise amino acids 41, 42, 81, 82, 85, 87, 110, 118, 151, 152, 155, 156, 157, 162, 163, 164, 165, 166, and 168 of one NS5A monomer, and amino acids 37, 38, 39 and 58 of both monomers, wherein each NS5A monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In still another embodiment, the amino acid resides that form the interaction area comprise amino acids 81, 85, 110, 152, 155, 165, and 168 of one NS5A monomer, and amino acids 37, 38, 39, 40 and 58 of both NS5A monomers, wherein each NS5A monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In still another embodiment, the amino acid resides that form the interaction area comprise amino acids 41, 42, 81, 82, 85, 87, 110, 118, 151, 152, 155, 156, 157, 162, 163, 164, 165, 166, and 168 of one NS5A monomer, and amino acids 37, 38, 39, 40 and 58 of both NS5A monomers, wherein each NS5A monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In yet another embodiment, the amino acid resides that form the interaction area comprise amino acids 81, 85, 110, 152, 155, 165, and 168 of one NS5A monomer, and amino acids 37, 38, 39 and 58 of both NS5A monomers, wherein each NS5A monomer comprises amino acids 37-168 of SEQ ID NO: 1, 2, 3, 4, 5, 6, 7 or 8. Preferably, each NS5A monomer comprises amino acids 36-198 of SEQ ID NO: 1, 2, 3, 4, 5, 6 or 8 or amino acids 36-197 of SEQ ID NO:7. In another embodiment, the amino acid resides that form the interaction area comprise amino acids 41, 42, 81, 82, 85, 87, 110, 118, 151, 152, 155, 156, 157, 162, 163, 164, 165, 166, and 168 of one NS5A monomer, and amino acids 37, 38, 39 and 58 of both monomers, wherein each NS5A monomer comprises amino acids 37-168 of SEQ ID NO: 1, 2, 3, 4, 5, 6, 7 or 8. Preferably, each NS5A monomer comprises amino acids 36-198 of SEQ ID NO: 1, 2, 3, 4, 5, 6 or 8 or amino acids 36-197 of SEQ ID NO:7. In still another embodiment, the amino acid resides that form the interaction area comprise amino acids 81, 85, 110, 152, 155, 165, and 168 of one NS5A monomer, and amino acids 37, 38, 39, 40 and 58 of both NS5A monomers, wherein each NS5A monomer comprises amino acids 37-168 of SEQ ID NO: 1, 2, 3, 4, 5, 6, 7 or 8. Preferably, each NS5A monomer comprises amino acids 36-198 of SEQ ID NO: 1, 2, 3, 4, 5, 6 or 8 or amino acids 36-197 of SEQ ID NO:7. In still another embodiment, the amino acid resides that form the interaction area comprise amino acids 41, 42, 81, 82, 85, 87, 110, 118, 151, 152, 155, 156, 157, 162, 163, 164, 165, 166, and 168 of one NS5A monomer, and amino acids 37, 38, 39, 40 and 58 of both NS5A monomers, wherein each NS5A monomer comprises amino acids 37-168 of SEQ ID NO: 1, 2, 3, 4, 5, 6, 7 or 8. Preferably, each NS5A monomer comprises amino acids 36-198 of SEQ ID NO: 1, 2, 3, 4, 5, 6 or 8 or amino acids 36-197 of SEQ ID NO:7. In the above embodiments, each of the two monomers in the NS5A dimer preferably consists of amino acids 37-168 of SEQ ID NO: 1, 2, 3, 4, 5, 6, 7 or 8, or amino acids 36-198 of SEQ ID NO: 1, 2, 3, 4, 5, 6 or 8 or amino acids 36-197 of SEQ ID NO:7, and the two monomers preferably have the same amino acid sequence.

In still another aspect, the present invention features still another method of identifying NS5A inhibitors. The method comprises docking a compound to an NS5A dimer using a computer docking program, wherein the NS5A dimer comprises two NS5A monomers and an interaction area, and the amino acid residues that form the interaction area comprise amino acids 81, 85, 110, 152, 155, 165, and 168 of both monomers. That at least a portion of the compound fits to the interaction area is indicative that the compound is an NS5A inhibitor. Preferably, the amino acid residues that form the interaction area comprise amino acids 81, 85, 110, 152, 155, 165, and 168 of one monomer, and amino acids 41, 42, 81, 82, 85, 87, 110, 118, 151, 152, 155, 156, 157, 162, 163, 164, 165, 166, and 168 of the other monomer. More preferably, the amino acid residues that form the interaction area comprise amino acids 41, 42, 81, 82, 85, 87, 110, 118, 151, 152, 155, 156, 157, 162, 163, 164, 165, 166, and 168 of both monomers. Because the interaction area includes sites from both monomers, a compound identified according to this aspect of the invention often has a dimeric or dimeric-like structure, with one part of the compound fitting to the interaction sites provided by the first NS5A monomer and the other part fitting to the interaction sites provided by the second NS5A monomer. The two parts of the compound can be the same or contain different moieties. In one embodiment, the compound has a symmetrical structure and consists of two identical moieties, with one moiety fitting to the interaction sites provided by the first NS5A monomer and the other identical moiety fitting to the interaction sites provided by the second NS5A. In one embodiment, a substantial portion of the compound of interest fits to this interaction area. In another embodiment, the whole compound fits or is docked to this interaction area.

In one embodiment of this aspect of the invention, the amino acid resides that form the interaction area comprise amino acids 81, 85, 110, 152, 155, 165, and 168 of both NS5A monomers, wherein each NS5A monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In another embodiment, the amino acid resides that form the interaction area comprise amino acids amino acids 81, 85, 110, 152, 155, 165 and 168 of one monomer, and amino acids 41, 42, 81, 82, 85, 87, 110, 118, 151, 152, 155, 156, 157, 162, 163, 164, 165, 166 and 168 of the other monomer, wherein each NS5A monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In still another embodiment, the amino acid resides that form the interaction area comprise amino acids 41, 42, 81, 82, 85, 87, 110, 118, 151, 152, 155, 156, 157, 162, 163, 164, 165, 166, and 168 of both monomers, wherein each NS5A monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In still another embodiment, the amino acid resides that form the interaction area comprise amino acids 81, 85, 110, 152, 155, 165, and 168 of both NS5A monomers, wherein each NS5A monomer comprises amino acids 37-168 of SEQ ID NO: 1, 2, 3, 4, 5, 6, 7 or 8. Preferably, each NS5A monomer comprises amino acids 36-198 of SEQ ID NO: 1, 2, 3, 4, 5, 6 or 8 or amino acids 36-197 of SEQ ID NO:7. In another embodiment, the amino acid resides that form the interaction area comprise amino acids 81, 85, 110, 152, 155, 165 and 168 of one monomer, and amino acids 41, 42, 81, 82, 85, 87, 110, 118, 151, 152, 155, 156, 157, 162, 163, 164, 165, 166 and 168 of the other monomer, wherein each NS5A monomer comprises amino acids 37-168 of SEQ ID NO: 1, 2, 3, 4, 5, 6, 7 or 8. Preferably, each NS5A monomer comprises amino acids 36-198 of SEQ ID NO: 1, 2, 3, 4, 5, 6 or 8 or amino acids 36-197 of SEQ ID NO:7. In still another embodiment, the amino acid resides that form the interaction area comprise amino acids 41, 42, 81, 82, 85, 87, 110, 118, 151, 152, 155, 156, 157, 162, 163, 164, 165, 166, and 168 of both monomers, wherein each NS5A monomer comprises amino acids 37-168 of SEQ ID NO: 1, 2, 3, 4, 5, 6, 7 or 8. Preferably, each NS5A monomer comprises amino acids 36-198 of SEQ ID NO: 1, 2, 3, 4, 5, 6 or 8 or amino acids 36-197 of SEQ ID NO:7. In the above embodiments, each of the two monomers in the NS5A dimer preferably consists of amino acids 37-168 of SEQ ID NO: 1, 2, 3, 4, 5, 6, 7 or 8, or amino acids 36-198 of SEQ ID NO: 1, 2, 3, 4, 5, 6 or 8 or amino acids 36-197 of SEQ ID NO:7, and the two monomers preferably have the same amino acid sequence.

In still yet another aspect, the present invention features yet another method of identifying NS5A inhibitors. The method comprises docking a compound to an NS5A dimer using a computer docking program, wherein the NS5A dimer comprises two NS5A monomers and an interaction area, and the amino acid residues that form the interaction area comprise amino acids 37, 38, 39, 58, 81, 85, 110, 152, 155, 165, and 168 of both monomers. That at least a portion of the compound fits to the interaction area is indicative that the compound is an NS5A inhibitor. Preferably, the amino acid residues that form the interaction area comprise amino acids 37, 38, 39, 40, 58, 81, 85, 110, 152, 155, 165, and 168 of both monomers. More preferably, the amino acid residues that form the interaction area comprise amino acids 37, 38, 39, 41, 42, 58, 81, 82, 85, 87, 110, 118, 151, 152, 155, 156, 157, 162, 163, 164, 165, 166, and 168 of both monomers. Highly preferably, the amino acid residues that form the interaction area comprise amino acids 37, 38, 39, 40, 41, 42, 58, 81, 82, 85, 87, 110, 118, 151, 152, 155, 156, 157, 162, 163, 164, 165, 166, and 168 of both monomers. Because the interaction area includes sites from both monomers, a compound identified according to this aspect of the invention often has a dimeric or dimeric-like structure and comprises at least three moieties. The first moiety fits to the interaction site formed by amino acids 81, 85, 110, 152, 155, 165, and 168 (or preferably, by amino acids 41, 42, 81, 82, 85, 87, 110, 118, 151, 152, 155, 156, 157, 162, 163, 164, 165, 166, and 168) of the first NS5A monomer, the second moiety fits to the interaction site formed by amino acids 81, 85, 110, 152, 155, 165, and 168 (or preferably, by amino acids 41, 42, 81, 82, 85, 87, 110, 118, 151, 152, 155, 156, 157, 162, 163, 164, 165, 166, and 168) of the second NS5A monomer, and the third moiety fits to the region formed by amino acids 37, 38, 39 and 58 (or preferably, by amino acids 37, 38, 39, 40 and 58) of both monomers. The first and second moieties can be the same or different. In one embodiment, a substantial portion of the compound of interest fits to this interaction area. In another embodiment, the whole compound fits or is docked to this interaction area.

In one embodiment of this aspect of the invention, the amino acid resides that form the interaction area comprise amino acids 37, 38, 39, 58, 81, 85, 110, 152, 155, 165, and 168 of both NS5A monomers, wherein each monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In another embodiment, the amino acid resides that form the interaction area comprise amino acids amino acids 37, 38, 39, 40, 58, 81, 85, 110, 152, 155, 165, and 168 of both NS5A monomers, wherein each monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In still another embodiment, the amino acid resides that form the interaction area comprise amino acids 37, 38, 39, 41, 42, 58, 81, 82, 85, 87, 110, 118, 151, 152, 155, 156, 157, 162, 163, 164, 165, 166, and 168 of both monomers, wherein each NS5A monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In still yet another embodiment, the amino acid resides that form the interaction area comprise amino acids 37, 38, 39, 40, 41, 42, 58, 81, 82, 85, 87, 110, 118, 151, 152, 155, 156, 157, 162, 163, 164, 165, 166, and 168 of both monomers, wherein each monomer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In another embodiment, the amino acid resides that form the interaction area comprise amino acids 37, 38, 39, 58, 81, 85, 110, 152, 155, 165, and 168 of both NS5A monomers, wherein each monomer comprises amino acids 37-168 of SEQ ID NO: 1, 2, 3, 4, 5, 6, 7 or 8. Preferably, each NS5A monomer comprises amino acids 36-198 of SEQ ID NO: 1, 2, 3, 4, 5, 6 or 8 or amino acids 36-197 of SEQ ID NO:7. In yet another embodiment, the amino acid resides that form the interaction area comprise amino acids amino acids 37, 38, 39, 40, 58, 81, 85, 110, 152, 155, 165, and 168 of both NS5A monomers, wherein each monomer comprises amino acids 37-168 of SEQ ID NO: 1, 2, 3, 4, 5, 6, 7 or 8. Preferably, each NS5A monomer comprises amino acids 36-198 of SEQ ID NO: 1, 2, 3, 4, 5, 6 or 8 or amino acids 36-197 of SEQ ID NO:7. In still another embodiment, the amino acid resides that form the interaction area comprise amino acids 37, 38, 39, 41, 42, 58, 81, 82, 85, 87, 110, 118, 151, 152, 155, 156, 157, 162, 163, 164, 165, 166, and 168 of both monomers, wherein each NS5A monomer comprises amino acids 37-168 of SEQ ID NO: 1, 2, 3, 4, 5, 6, 7 or 8. Preferably, each NS5A monomer comprises amino acids 36-198 of SEQ ID NO: 1, 2, 3, 4, 5, 6 or 8 or amino acids 36-197 of SEQ ID NO:7. In still yet another embodiment, the amino acid resides that form the interaction area comprise amino acids 37, 38, 39, 40, 41, 42, 58, 81, 82, 85, 87, 110, 118, 151, 152, 155, 156, 157, 162, 163, 164, 165, 166, and 168 of both monomers, wherein each monomer comprises amino acids 37-168 of SEQ ID NO: 1, 2, 3, 4, 5, 6, 7 or 8. Preferably, each NS5A monomer comprises amino acids 36-198 of SEQ ID NO: 1, 2, 3, 4, 5, 6 or 8 or amino acids 36-197 of SEQ ID NO:7. In the above embodiments, each of the two monomers in the NS5A dimer preferably consists of amino acids 37-168 of SEQ ID NO: 1, 2, 3, 4, 5, 6, 7 or 8, or amino acids 36-198 of SEQ ID NO: 1, 2, 3, 4, 5, 6 or 8 or amino acids 36-197 of SEQ ID NO:7, and the two monomers preferably have the same amino acid sequence.

The methods of the present invention can further comprise detecting activity of an identified compound for inhibiting HCV replication in a cell culture, or detecting activity of an identified compound for inhibiting HCV infection or replication in an animal or a cell culture. Cell cultures capable of supporting the replication of HCV replicons or HCV viruses are well known in the art. See, for example, Bartenschlager, NATURE REVIEWS DRUG DISCOVERY 1:911-916 (2002), and Bartenschlager, CURRENT OPINION IN MICROBIOLOGY 9:416-422 (2006), both of which are incorporated herein by reference in their entireties. In one embodiment, the cell culture used in the present invention is a human hepatoma cell line. In another embodiment, the cell culture is a Huh-7 cell culture, such as a Huh7.5 or Huh7-Lunet cell culture. In still another embodiment, the cell culture is a Huh-6 cell culture. Suitable animal models for detecting anti-HCV activities of a compound of interest include chimpanzee, mouse, rat, marmoset, or tupaia models. See, e.g., Galun et al., J INFECT DIS 172:25-30 (1995), Xie et al., VIROLOGY 244:513-520 (1998), Mercer et al., NAT. MED. 7:927-933 (2001), Wu et al., GASTROENTEROLOGY 128:1416-1423 (2005), and Zhu et al., ANTIMICROB AGENTS CHEMOTHER. 50:3260-3268 (2006).

The anti-HCV activities of a compound of interest can be evaluated by a variety of assays known in the art. For instance, two stable subgenomic replicon cell lines can be used for compound characterization in cell culture: one derived from genotype 1a-H77 and the other derived from genotype 1b-Con1. The replicon constructs can be bicistronic subgenomic replicons. The genotype 1a replicon construct contains NS3-NS5B coding region derived from the H77 strain of HCV (1a-H77). The replicon also has a firefly luciferase reporter and a neomycin phosphotransferase (Neo) selectable marker. These two coding regions, separated by the FMDV 2a protease, comprise the first cistron of the bicistronic replicon construct, with the second cistron containing the NS3-NS5B coding region with addition of adaptive mutations. The 1b-Con1 replicon construct is identical to the 1a-H77 replicon, except that the NS3-NS5B coding region is derived from the 1b-Con1 strain and that the replicon contains different adaptive mutations. Replicon cell lines can be maintained in Dulbecco's modified Eagles medium (DMEM) containing 10% (v/v) fetal bovine serum (FBS), 100 IU/ml penicillin, 100 mg/ml streptomycin (Invitrogen), and 200 mg/ml G418 (Invitrogen).

The inhibitory effects of the compounds of the invention on HCV replication can be determined by measuring activity of the luciferase reporter gene. For example, replicon-containing cells can be seeded into 96 well plates at a density of 5000 cells per well in 100 μl DMEM containing 5% FBS. The following day compounds can be diluted in dimethyl sulfoxide (DMSO) to generate a 200× stock in a series of eight half-log dilutions. The dilution series can then be further diluted 100-fold in the medium containing 5% FBS. Medium with the inhibitor is added to the overnight cell culture plates already containing 100 μl of DMEM with 5% FBS. In assays measuring inhibitory activity in the presence of human plasma, the medium from the overnight cell culture plates can be replaced with DMEM containing 40% human plasma and 5% FBS. The cells can be incubated for three days in the tissue culture incubators and are then lysed for RNA extraction. For the luciferase assay, 30 μl of Passive Lysis buffer (Promega) can be added to each well, and then the plates are incubated for 15 minutes with rocking to lyse the cells. Luciferin solution (100 μl, Promega) can be added to each well, and luciferase activity can be measured with a Victor II luminometer (Perkin-Elmer). The percent inhibition of WY RNA replication can be calculated for each compound concentration and the IC₅₀ and/or EC₅₀ value can be calculated using nonlinear regression curve fitting to the 4-parameter logistic equation and GraphPad Prism 4 software.

The present invention also feature compounds identified according to the methods of the present invention. In addition, the present invention features compounds created by rational drug design based on the interaction sites identified by the present invention. The compounds of the present invention can be docked to an NS5A dimer using a computer docking program and can inhibit the activity of NS5A or HCV replication. Preferably, a compound identified by the present invention has an IC50 value of no more than 1 μM for inhibiting HCV replication when measured in a standard HCV replicon assay, such as those described above. More preferably, a compound thus identified has an IC50 value of no more than 100 nM for inhibiting HCV replication when measured in a standard HCV replicon assay. Highly preferably, a compound thus identified has an IC50 value of no more than 10 nM for inhibiting HCV replication when measured in a standard HCV replicon assay. Most preferably, a compound thus identified has an IC50 value of no more than 1 nM for inhibiting HCV replication when measured in a standard HCV replicon assay. In one embodiment, the HCV replicon assay uses a HCV-1a replicon (e.g., a HCV 1a-H77 replicon) and the inhibitory activity is measured in the presence of 40% plasma. In another embodiment, the HCV replicon assay uses a HCV-1b replicon (e.g., a HCV 1b-Con1 replicon) and the inhibitory activity is measured in the presence of 40% plasma.

In one aspect, at least part of a compound of the present invention fits to an interaction site on an NS5A dimer, wherein the amino acid residues that form the interaction site comprise amino acids 81, 85, 110, 152, 155, 165, and 168 of one monomer in the NS5A dimer. Preferably, the amino acid residues that form the interaction area comprise amino acids 41, 42, 81, 82, 85, 87, 110, 118, 151, 152, 155, 156, 157, 162, 163, 164, 165, 166, and 168 of one monomer in the NS5A dimer. In one embodiment, each monomer in the NS5A dimer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In another embodiment, each monomer in the NS5A dimer comprises amino acids 37-168 of SEQ ID NO: 1, 2, 3, 4, 5, 6, 7 or 8, or preferably, comprises amino acids 36-198 of SEQ ID NO: 1, 2, 3, 4, 5, 6 or 8 or amino acids 36-197 of SEQ ID NO:7. In a further embodiment, each monomer in the NS5A dimer consists of amino acids 37-168 of SEQ ID NO: 1, 2, 3, 4, 5, 6, 7 or 8, or preferably amino acids 36-198 of SEQ ID NO: 1, 2, 3, 4, 5, 6 or 8 or amino acids 36-197 of SEQ ID NO:7. In this aspect of the invention, a substantial portion of the compound, or the whole compound, preferably fits to this interaction site. The two monomers in the NS5A dimer preferably have the same amino acid sequence.

Non-limiting examples of the compounds of this aspect of the invention include many compounds (e.g., Examples 1-50) described in U.S. Patent Application Publication No. 20070232645, the entire content of which is incorporated herein by reference. Non-limiting examples of the compounds of this aspect of the invention also include those described in Table 1.

In still another aspect, at least part of a compound of the present invention fits to an interaction site on an NS5A dimer, wherein the amino acid residues that form the interaction site comprise amino acids 81, 85, 110, 152, 155, 165, and 168 of one monomer, and amino acids 37, 38, 39 and 58 of both monomers of the NS5A dimer. Preferably, the amino acid residues that form the interaction site comprise amino acids 81, 85, 110, 152, 155, 165, and 168 of one monomer, and 37, 38, 39, 40 and 58 of both monomers. Also preferably, the amino acid residues that form the interaction site comprise amino acids 41, 42, 81, 82, 85, 87, 110, 118, 151, 152, 155, 156, 157, 162, 163, 164, 165, 166, and 168 of one monomer, and amino acids 37, 38, 39 and 58 of both monomers. More preferably, the amino acid residues that form the interaction site comprise amino acids 41, 42, 81, 82, 85, 87, 110, 118, 151, 152, 155, 156, 157, 162, 163, 164, 165, 166, and 168 of one monomer, and amino acids 37, 38, 39, 40 and 58 of both monomers. In one embodiment, each monomer in the NS5A dimer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In another embodiment, each monomer comprises amino acids 37-168 of SEQ ID NO: 1, 2, 3, 4, 5, 6, 7 or 8, or preferably amino acids 36-198 of SEQ ID NO: 1, 2, 3, 4, 5, 6 or 8 or amino acids 36-197 of SEQ ID NO:7. In yet another embodiment, each monomer consists of amino acids 37-168 of SEQ ID NO: 1, 2, 3, 4, 5, 6, 7 or 8, or amino acids 36-198 of SEQ ID NO: 1, 2, 3, 4, 5, 6 or 8 or amino acids 36-197 of SEQ ID NO:7. In this aspect of the invention, a substantial portion of the compound, or the whole compound, preferably fits to the interaction site. The two monomers in the NS5A dimer preferably have the same amino acid sequence.

Non-limiting examples of the compounds of this aspect of the invention include many compounds (e.g., Examples 1-50) described in U.S. Patent Application Publication No. 20070232627, the entire content of which is incorporated herein by reference. Non-limiting examples of the compounds of this aspect of the invention also include those described in Tables 2 and 3.

In still another aspect, at least part of a compound of the present invention fits to an interaction site on an NS5A dimer, wherein the amino acid residues that form the interaction site comprise amino acids 81, 85, 110, 152, 155, 165, and 168 of both monomers in the NS5A dimer. Preferably, the amino acid residues that form the interaction site comprise amino acids 81, 85, 110, 152, 155, 165, and 168 of one monomer, and amino acids 41, 42, 81, 82, 85, 87, 110, 118, 151, 152, 155, 156, 157, 162, 163, 164, 165, 166, and 168 of the other monomer. More preferably, the amino acid residues that form the interaction site comprise amino acids 41, 42, 81, 82, 85, 87, 110, 118, 151, 152, 155, 156, 157, 162, 163, 164, 165, 166, and 168 of both monomers. Because the interaction site includes areas from both monomers, a compound of this aspect of the invention often has a dimeric or dimeric-like structure, with one part of the compound fitting to the interaction area provided by the first NS5A monomer and the other part fitting to the interaction area provided by the second NS5A monomer. The two parts of the compound can be the same or contain different moieties. For example, the compound can have a symmetrical structure and consists of two identical moieties, with one moiety fitting to the interaction area provided by the first NS5A monomer and the other identical moiety fitting to the interaction area provided by the second NS5A. In one embodiment, each monomer in the NS5A dimer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In another embodiment, each monomer in the NS5A dimer comprises amino acids 37-168 of SEQ ID NO: 1, 2, 3, 4, 5, 6, 7 or 8, or preferably, amino acids 36-198 of SEQ ID NO: 1, 2, 3, 4, 5, 6 or 8 or amino acids 36-197 of SEQ ID NO:7. In still another embodiment, each monomer in the NS5A dimer consists of amino acids 37-168 of SEQ ID NO: 1, 2, 3, 4, 5, 6, 7 or 8, or amino acids 36-198 of SEQ ID NO: 1, 2, 3, 4, 5, 6 or 8 or amino acids 36-197 of SEQ ID NO:7. In this aspect of the invention, a substantial portion of the compound, or the whole compound, preferably fits to the interaction site. The two monomers in the NS5A dimer preferably have the same amino acid sequence.

Non-limiting examples of the compounds of this aspect of the invention include many compounds described in U.S. Patent Application Ser. Nos. 61/140,318 (David A. DeGoey et al. entitled “Anti-viral Compounds” and filed Dec. 23, 2008) and 61/140,262 (David A. DeGoey et al. entitled “Anti-viral Compounds” and filed Dec. 23, 2008) and U.S. Patent Application Publication Nos. 20080299075, 20090068140 and 20090202478, all of which are incorporated herein by reference in their entireties. Specific examples of the compounds of this aspect of the invention are listed in Table 1.

TABLE 1

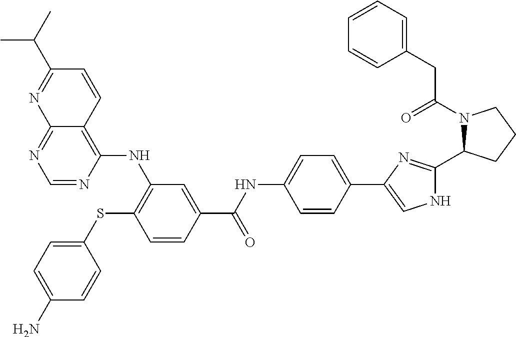

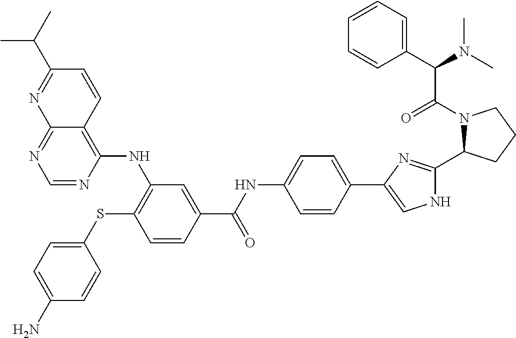

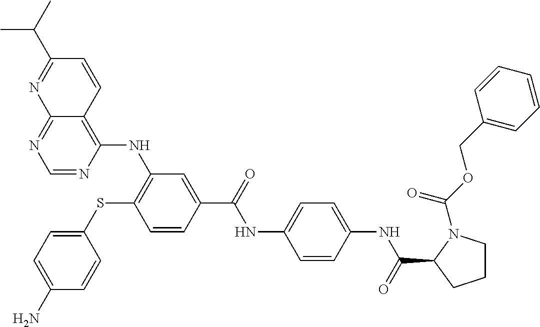

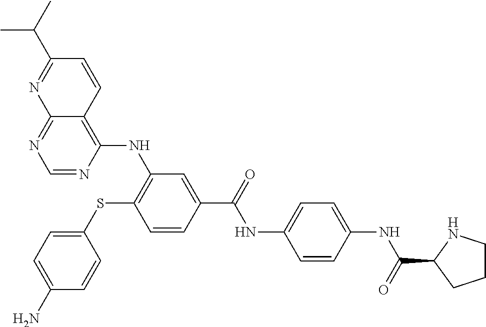

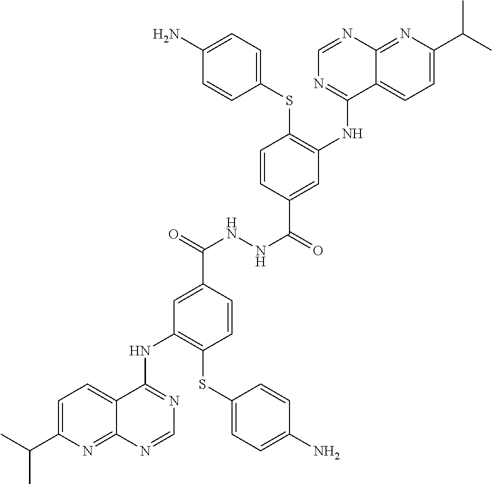

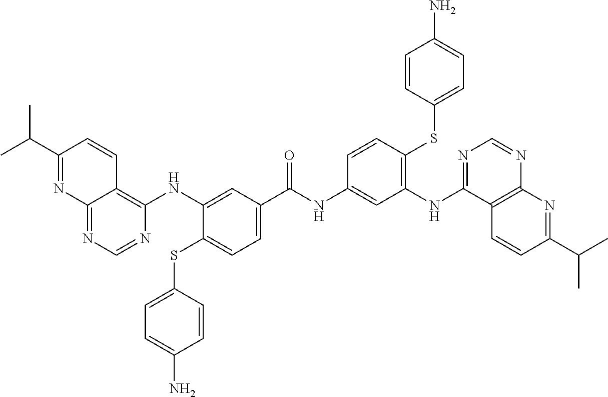

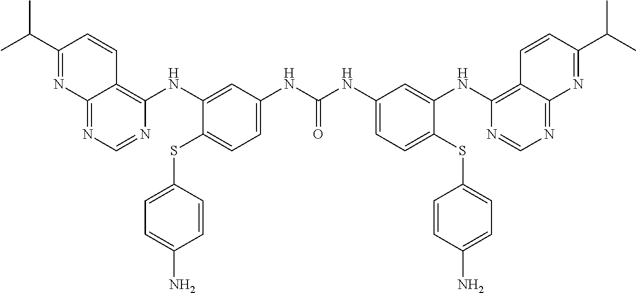

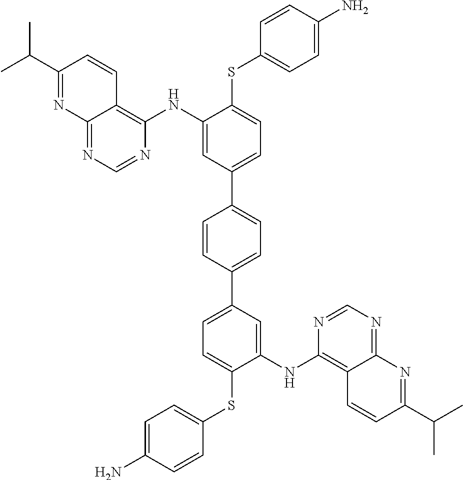

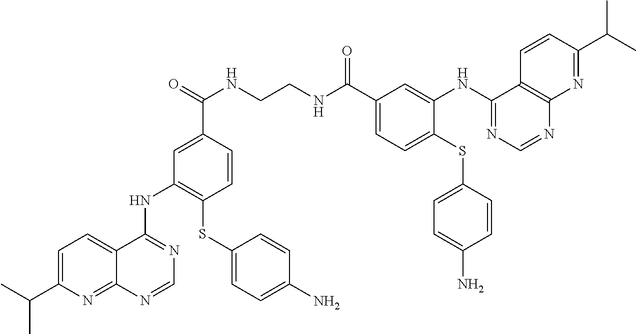

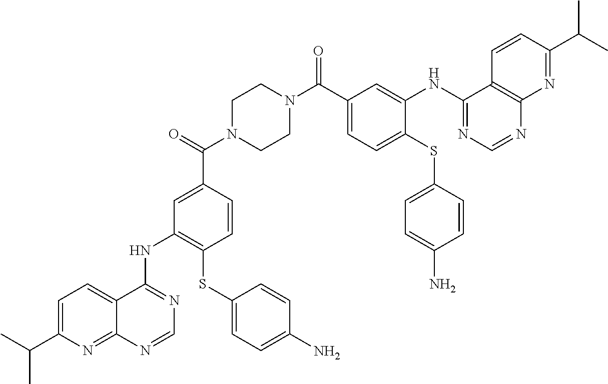

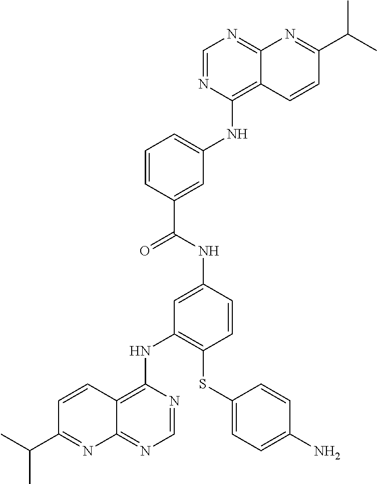

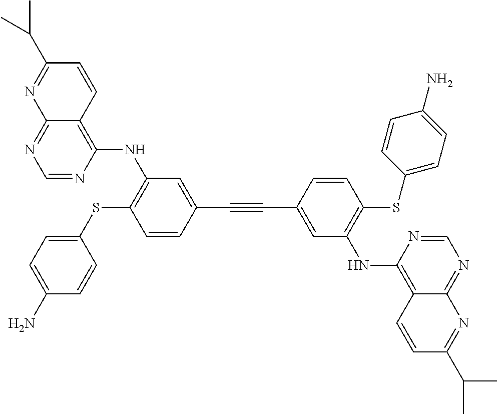

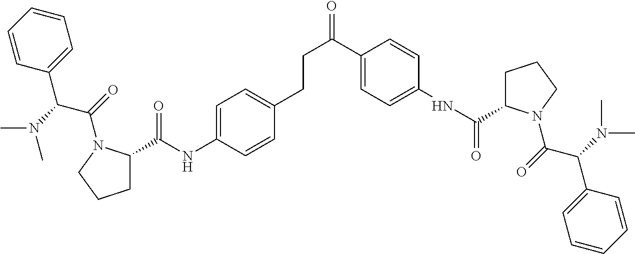

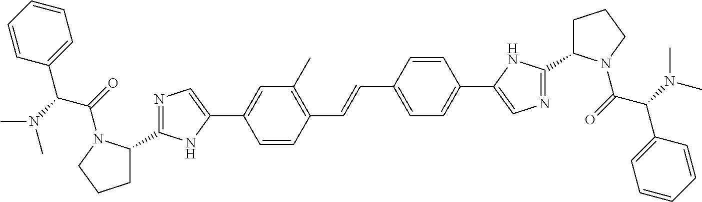

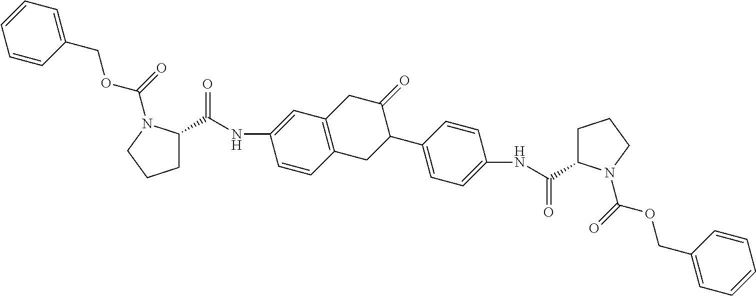

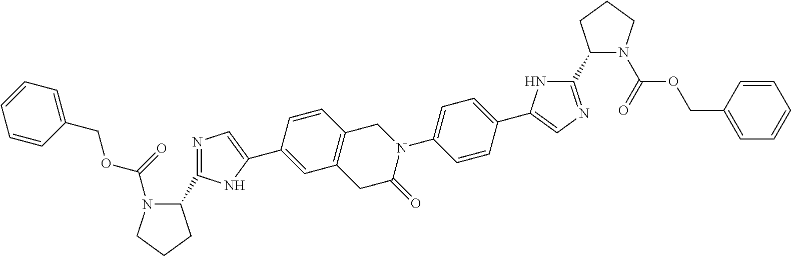

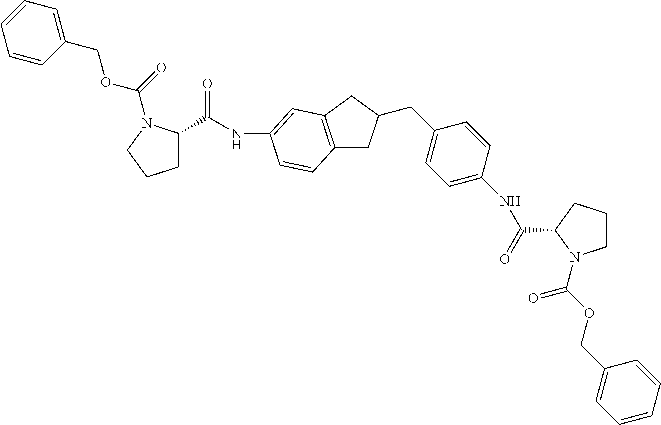

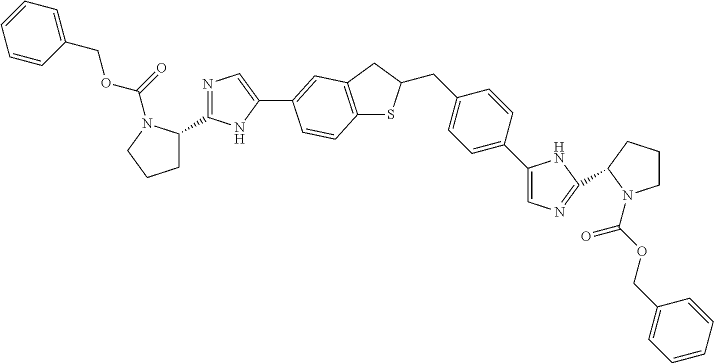

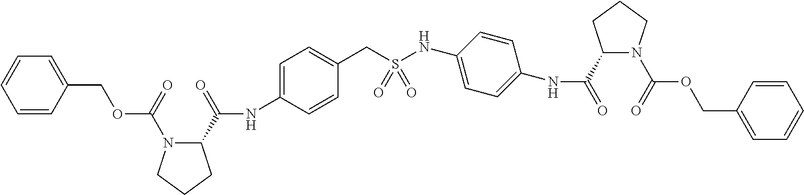

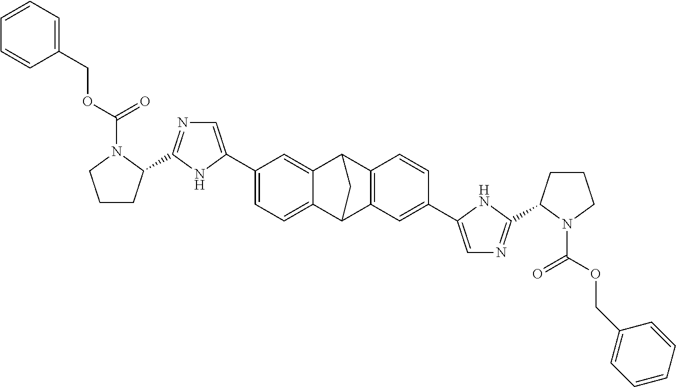

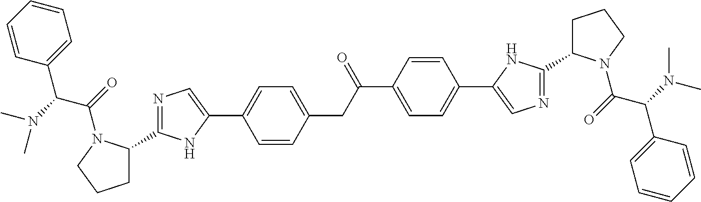

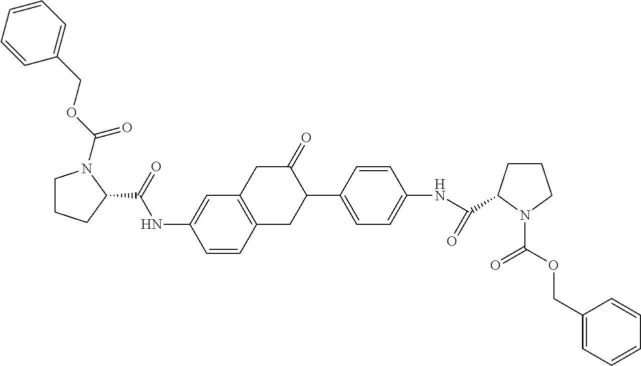

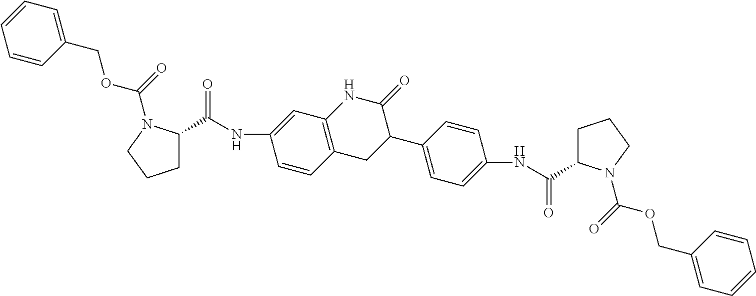

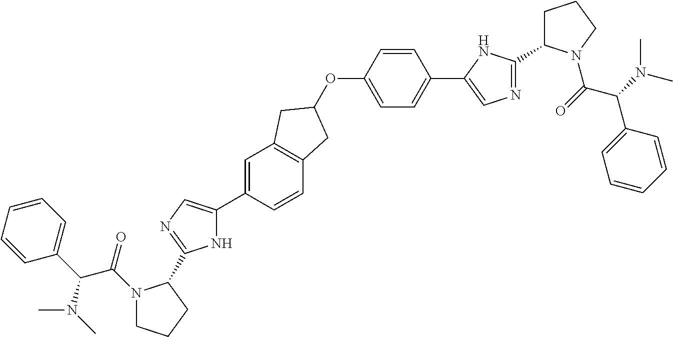

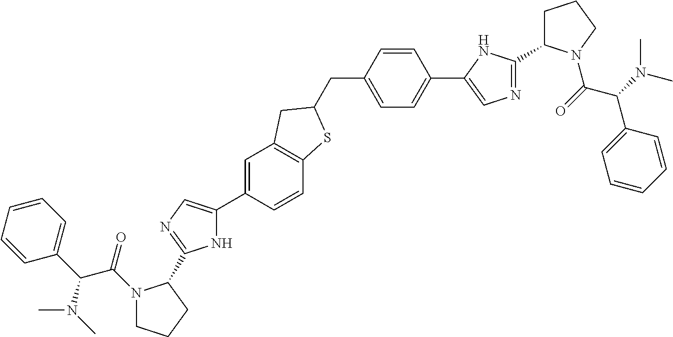

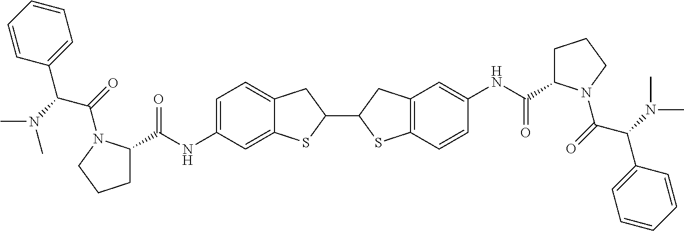

In still yet another aspect, at least part of a compound of the present invention fits to an interaction site on an NS5A dimer, wherein the amino acid residues that form the interaction site comprise amino acids 37, 38, 39, 58, 81, 85, 110, 152, 155, 165, and 168 of both monomers. Preferably, the amino acid residues that form the interaction site comprise amino acids 37, 38, 39, 40, 58, 81, 85, 110, 152, 155, 165, and 168 of both monomers. More preferably, the amino acid residues that form the interaction site comprise amino acids 37, 38, 39, 41, 42, 58, 81, 82, 85, 87, 110, 118, 151, 152, 155, 156, 157, 162, 163, 164, 165, 166, and 168 of both monomers. Highly preferably, the amino acid residues that form the interaction site comprise amino acids 37, 38, 39, 40, 41, 42, 58, 81, 82, 85, 87, 110, 118, 151, 152, 155, 156, 157, 162, 163, 164, 165, 166, and 168 of both monomers. Because the interaction site includes areas provided by both monomers, a compound of this aspect of the invention often has a dimeric or dimeric-like structure and comprises at least three moieties. The first moiety fits to the interaction area formed by amino acids 81, 85, 110, 152, 155, 165, and 168 (or preferably, by amino acids 41, 42, 81, 82, 85, 87, 110, 118, 151, 152, 155, 156, 157, 162, 163, 164, 165, 166, and 168) of the first NS5A monomer, the second moiety fits to the interaction area formed by amino acids 81, 85, 110, 152, 155, 165, and 168 (or preferably, by amino acids 41, 42, 81, 82, 85, 87, 110, 118, 151, 152, 155, 156, 157, 162, 163, 164, 165, 166, and 168) of the second NS5A monomer, and third moiety fits to the region formed by amino acids 37, 38, 39 and 58 (or preferably, by amino acids 37, 38, 39, 40 and 58) of both monomers. The first and second moieties can be the same or different, and the third moiety can be, without limitation, -L-E as described above. In one embodiment, each monomer in the NS5A dimer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In another embodiment, each monomer in the NS5A dimer comprises amino acids 37-168 of SEQ ID NO: 1, 2, 3, 4, 5, 6, 7 or 8, or preferably, amino acids 36-198 of SEQ ID NO: 1, 2, 3, 4, 5, 6 or 8 or amino acids 36-197 of SEQ ID NO:7. In yet another embodiment, each monomer in the NS5A dimer consists of amino acids 37-168 of SEQ ID NO: 1, 2, 3, 4, 5, 6, 7 or 8, or amino acids 36-198 of SEQ ID NO: 1, 2, 3, 4, 5, 6 or 8 or amino acids 36-197 of SEQ ID NO:7. In this aspect of the invention, a substantial portion of the compound, or the whole compound, preferably fits to the interaction site. The two monomers in the NS5A dimer preferably have the same amino acid sequence.

Non-limiting examples of the compounds of this aspect of the invention include those described in Tables 2 and 3.

TABLE 2

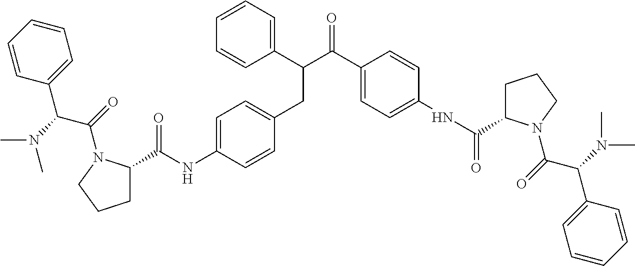

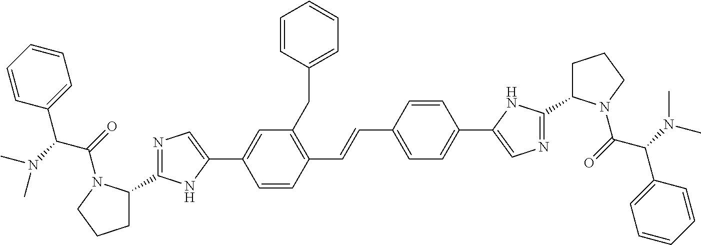

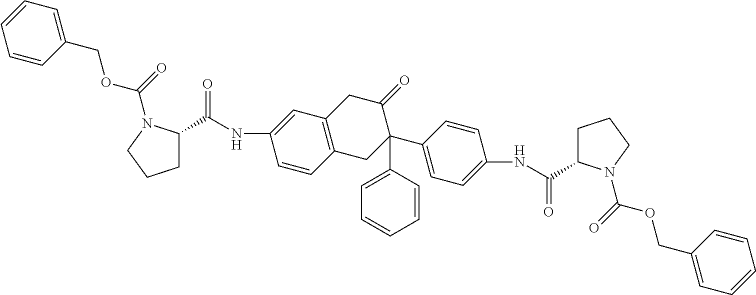

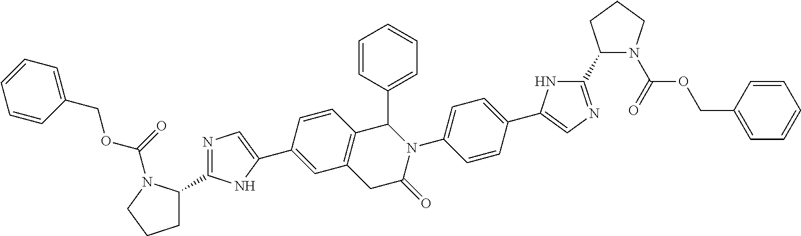

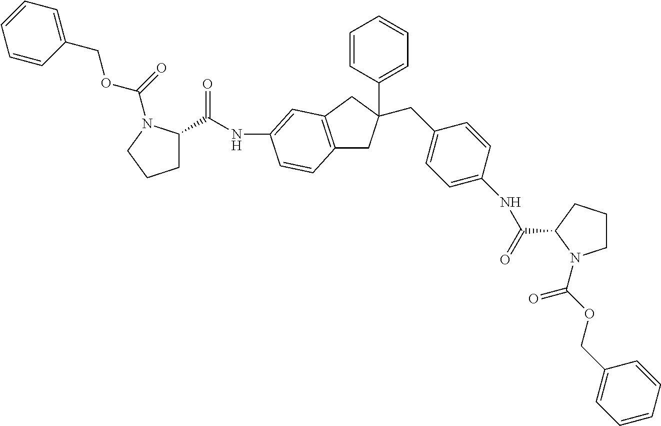

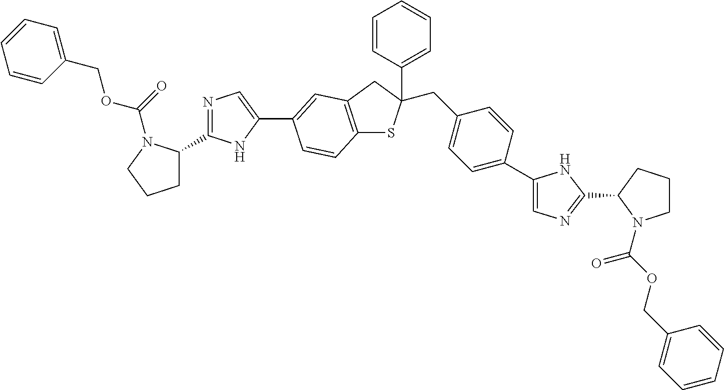

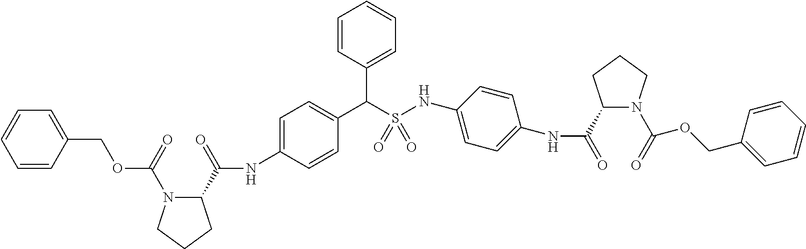

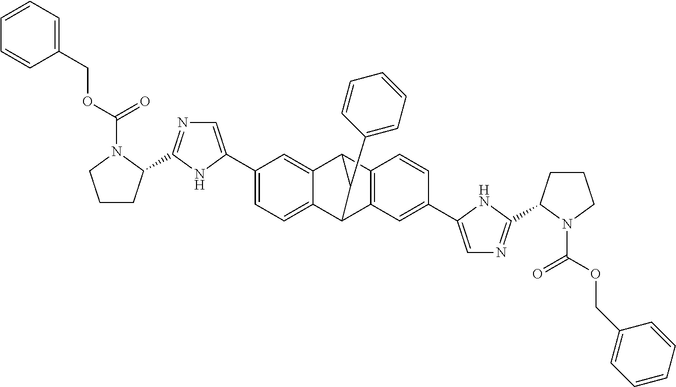

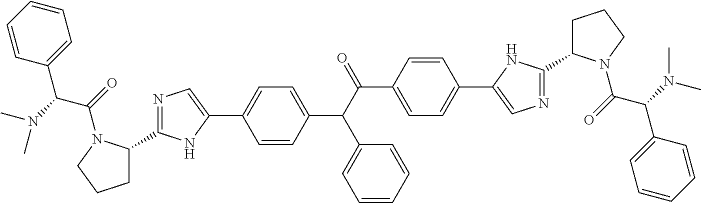

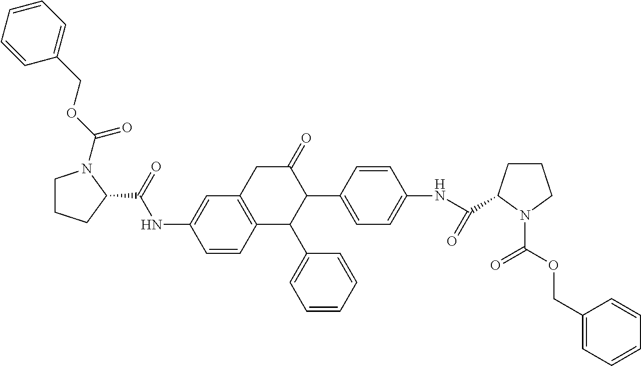

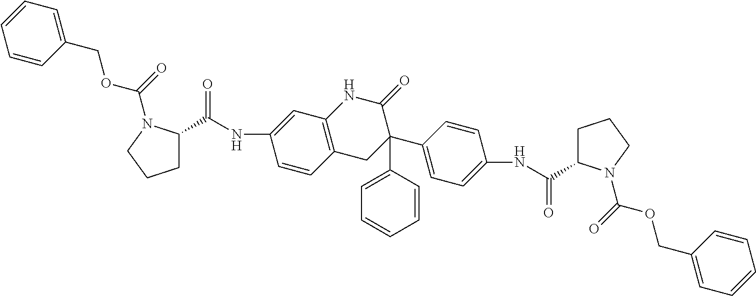

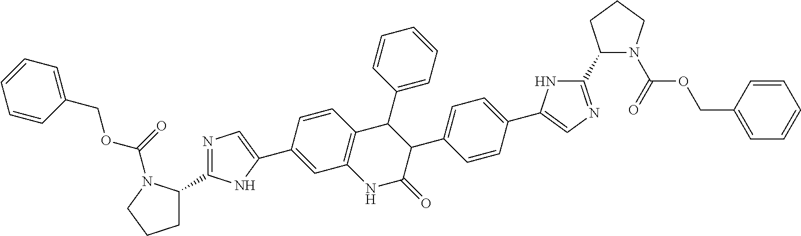

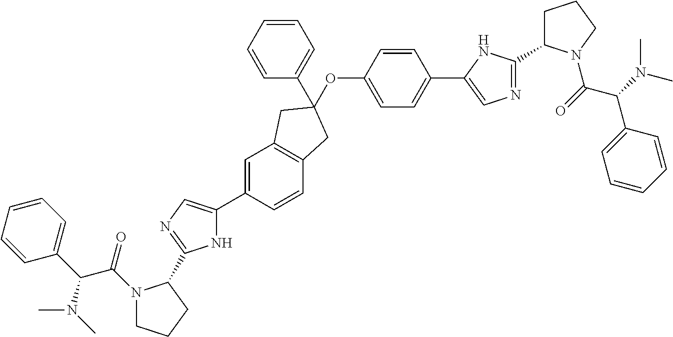

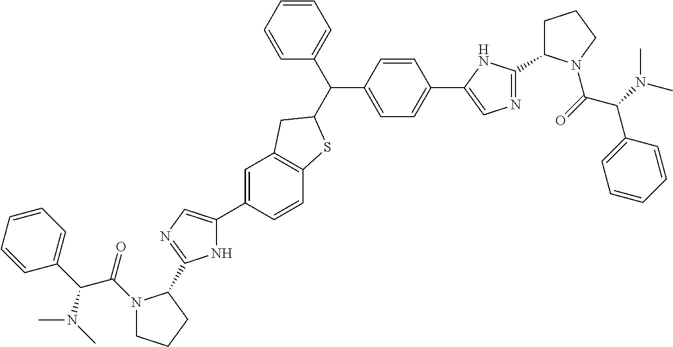

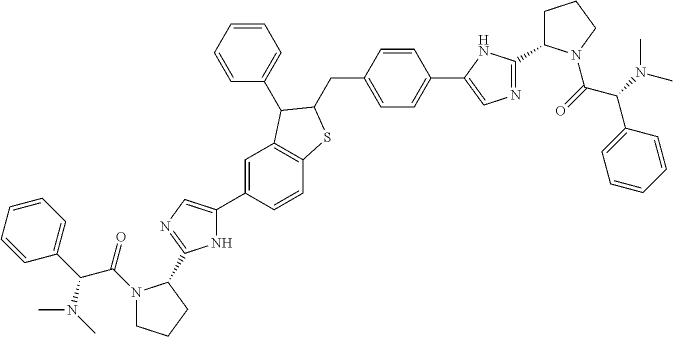

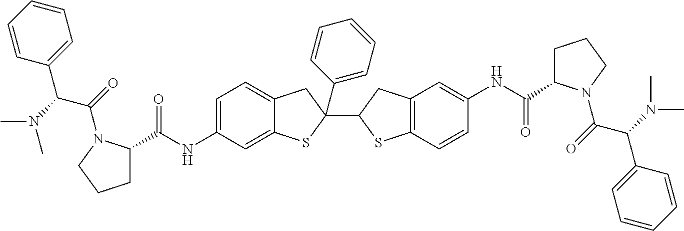

TABLE 3
(wherein R is —L—E as defined above)

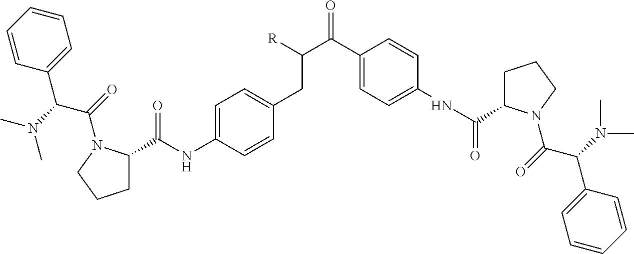

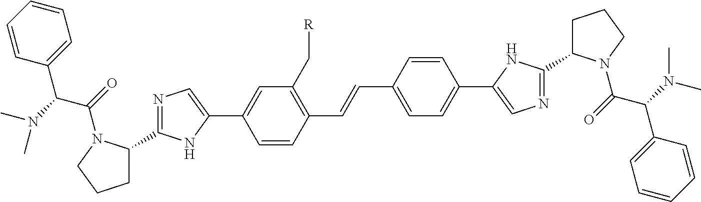

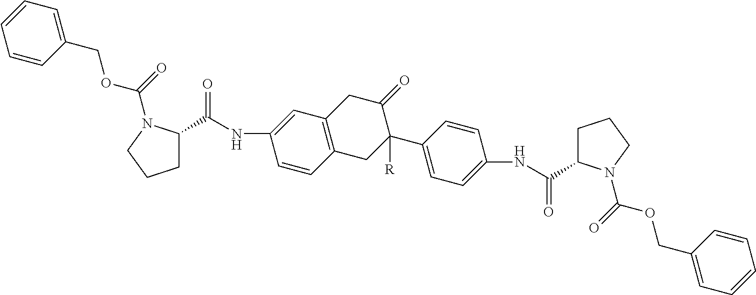

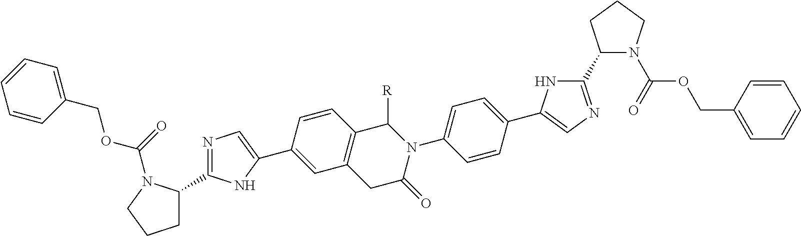

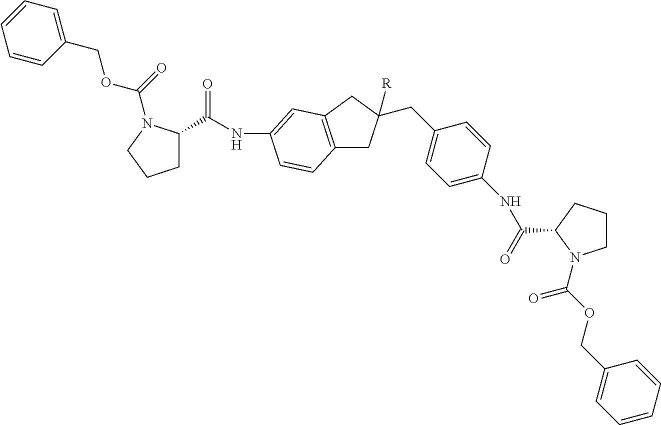

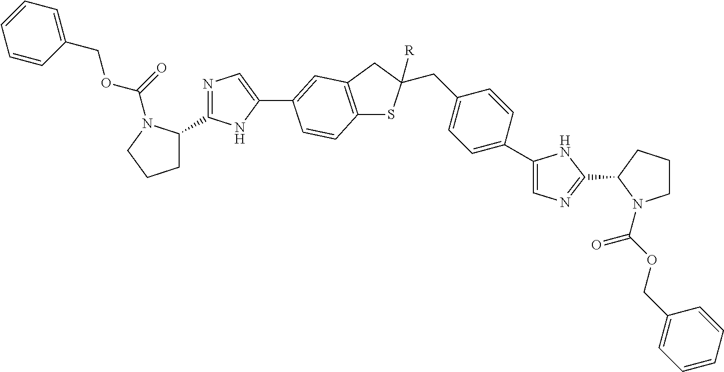

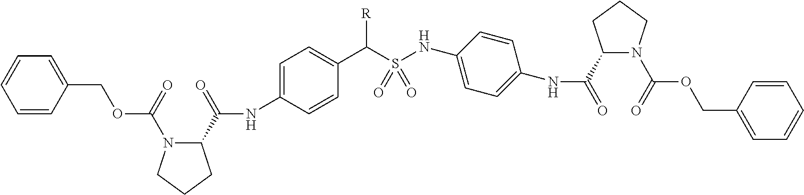

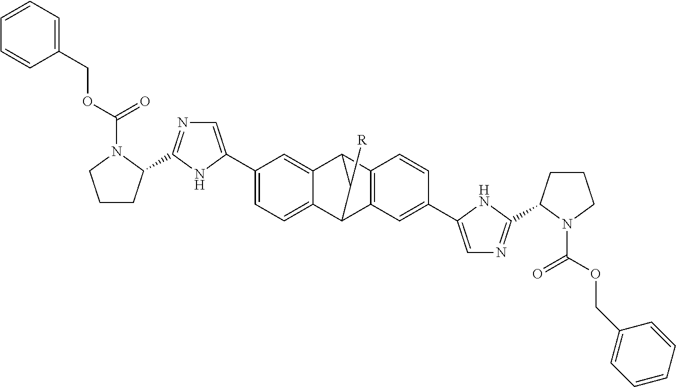

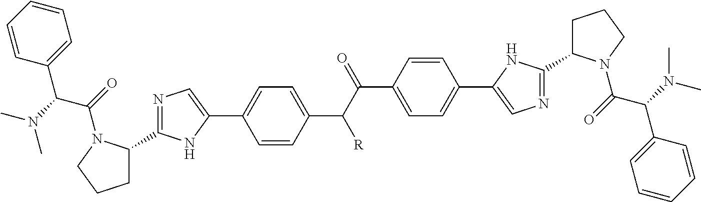

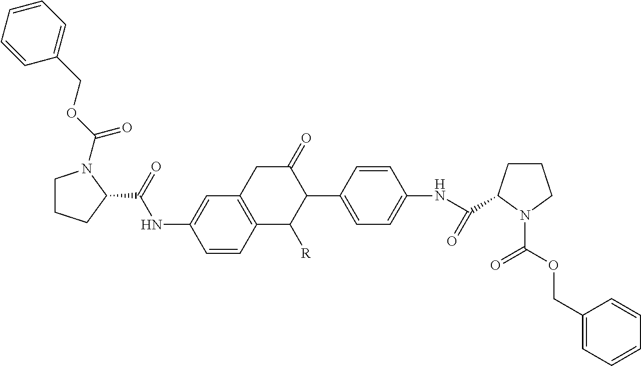

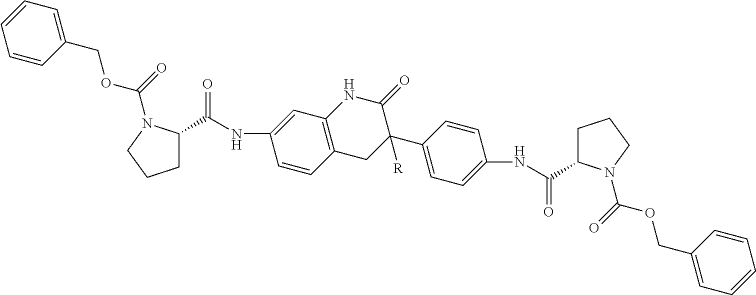

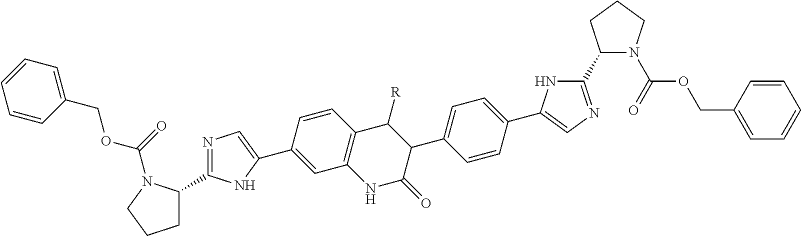

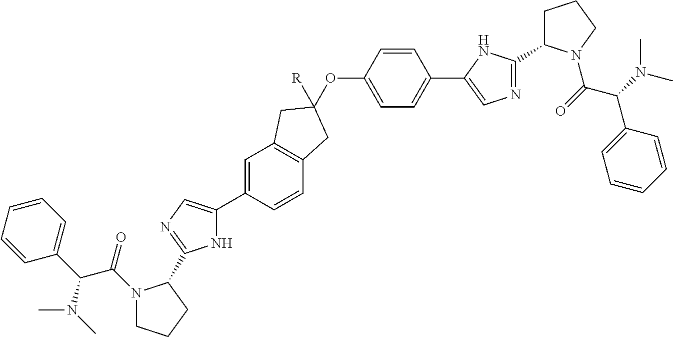

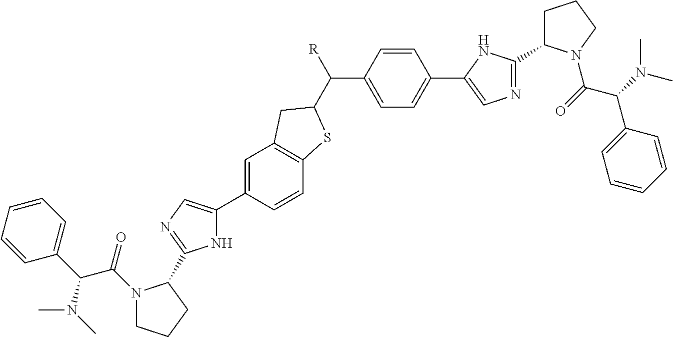

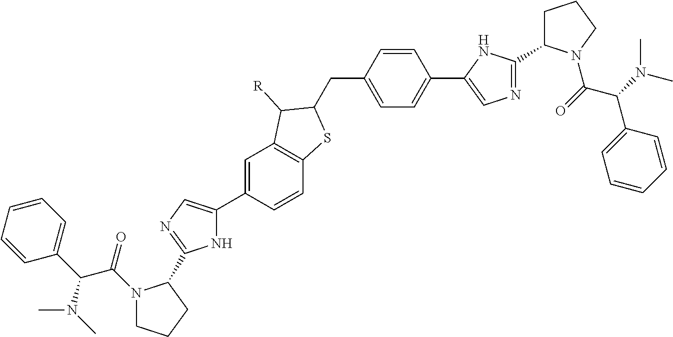

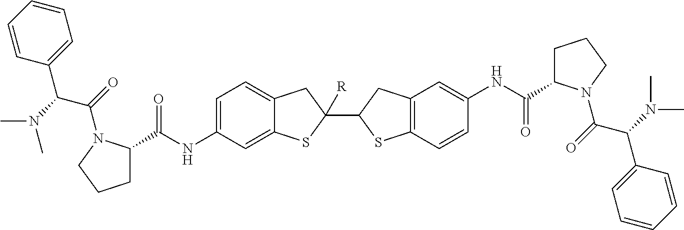

Preferably, R in Table 3 comprises C₅-C₆carbocycle, 5- to 6-membered heterocycle, or 6- to 12-membered bicycle, each of which is optionally substituted with one or more R_{A} as defined above. Also preferably, R comprises C₁-C₆alkyl, C₂-C₆alkenyl or C₂-C₆alkynyl, each of which is optionally substituted with one or more R_{L} as defined above. In one example, R comprises phenyl optionally substituted with one or more substituents selected from is halogen, hydroxy, mercapto, amino, carboxy, C₁-C₆alkyl, C₂-C₆alkenyl or C₂-C₆alkynyl, wherein each of said C₁-C₆alkyl, C₂-C₆alkenyl or C₂-C₆alkynyl is independently optionally substituted at each occurrence with one or more substituents selected from halogen, hydroxy, mercapto, amino or carboxy. In another example, R comprises C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl or cyano.

In yet another aspect, a compound of the present invention comprises a moiety (e.g., -L-E or R as described above) that fits to an interaction site on an NS5A dimer, wherein the amino acid residues that form the interaction site comprise amino acids 37, 38, 39 and 58 of both monomers in the NS5A dimer. Preferably, the amino acid residues that form the interaction site comprise amino acids 37, 38, 39, 40 and 58 of both monomers. More preferably, the interaction site has a pocket shape. In one embodiment, each NS5A monomer in the NS5A dimer comprises an NS5A sequence of HCV 1, 2, 3, 4, 5, 6, 7, 8, 9, 10, or 11 (e.g., HCV 1a, 1b, 1c, 2a, 2b, 2c, 3a, 3b, 4a, 4b, 4c, 4d, 4e, 5a, 6a, 7a, 7b, 8a, 8b, 9a, 10a or 11a). In another embodiment, each monomer in the NS5A dimer comprises amino acids 37-168 of SEQ ID NO: 1, 2, 3, 4, 5, 6, 7 or 8. Preferably, each monomer comprises amino acids 36-198 of SEQ ID NO: 1, 2, 3, 4, 5, 6 or 8 or amino acids 36-197 of SEQ ID NO:7. In yet another embodiments, each monomer consists of amino acids 37-168 of SEQ ID NO: 1, 2, 3, 4, 5, 6, 7 or 8, or amino acids 36-198 of SEQ ID NO: 1, 2, 3, 4, 5, 6 or 8 or amino acids 36-197 of SEQ ID NO:7. In this aspect of the invention, the two NS5A monomers in the NS5A dimer preferably have the same amino acid sequence.

Non-limiting examples of the compounds of this aspect of the invention include many compounds (e.g., Examples 1-50) described in U.S. Patent Application Publication No. 20070232627, as well as those described in Tables 2 and 3.

In still another aspect, a compound of the present invention comprises a moiety (e.g., -L-E as described above) that fits to an interaction site on an NS5A dimer, wherein the amino acid residues that form the interaction site comprise Phe 37, Ser 38, Cys 39 and Pro 58 of both monomers of the NS5A dimer. Preferably, the amino acid residues that form the interaction site comprise Phe 37, Ser 38, Cys 39, Gln 40 and Pro 58 of both monomers. More preferably, each monomer in the NS5A dimer comprises amino acid 37-168 of SEQ ID NO:1, or amino acids 36-198 of SEQ ID NO:1. Highly preferably, each monomer in the NS5A dimer consists of amino acids 37-168 of SEQ ID NO:1, or amino acids 36-198 of SEQ ID NO:1. In this aspect of the invention, the two monomers in the NS5A dimer preferably have the same amino acid sequence.

In another aspect, at least part of a compound of the present invention fits to an interaction site on an NS5A dimer, wherein the amino acid residues that form the interaction site comprise Ser 81, His 85, Leu 110, Glu 152, Gly 155, Cys 165, and Leu 168 of one monomer in the NS5A dimer. Preferably, the amino acid residues that form the interaction site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of the monomer. Preferably, each monomer in the NS5A dimer comprises amino acid 37-168 of SEQ ID NO:1, or amino acids 36-198 of SEQ ID NO:1. More preferably, each monomer in the NS5A dimer consists of amino acids 37-168 of SEQ ID NO:1, or amino acids 36-198 of SEQ ID NO:1. In this aspect of the invention, a substantial portion of the compound, or the whole compound, preferably fits to the interaction site. The two monomers in the NS5A dimer preferably have the same amino acid sequence.

In yet another aspect, at least part of a compound of the present invention fits to an interaction site on an NS5A dimer, wherein the amino acid residues that form the interaction site comprise Ser 81, His 85, Leu 110, Glu 152, Gly 155, Cys 165, and Leu 168 of one monomer, and Phe 37, Ser 38, Cys 39 and Pro 58 of both monomers in the NS5A dimer. Preferably, the amino acid residues that form the interaction site comprise Ser 81, His 85, Leu 110, Glu 152, Gly 155, Cys 165, and Leu 168 of one monomer, and Phe 37, Ser 38, Cys 39, Gln 40 and Pro 58 of both monomers. Also preferably, the amino acid residues that form the interaction site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of one monomer, and Phe 37, Ser 38, Cys 39 and Pro 58 of both monomers. More preferably, the amino acid residues that form the interaction site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of one monomer, and Phe 37, Ser 38, Cys 39, Gln 40 and Pro 58 of both monomers. Preferably, each monomer in the NS5A dimer comprises amino acid 37-168 of SEQ ID NO:1, or amino acids 36-198 of SEQ ID NO:1. More preferably, each monomer in the NS5A dimer consists of amino acids 37-168 of SEQ ID NO:1, or amino acids 36-198 of SEQ ID NO:1. In this aspect of the invention, a substantial portion of the compound, or the whole compound, preferably fits to the interaction site. The two monomers in the NS5A dimer preferably have the same amino acid sequence.

In still another aspect, at least part of a compound of the present invention fits to an interaction site on an NS5A dimer, wherein the amino acid residues that form the interaction site comprise Ser 81, His 85, Leu 110, Glu 152, Gly 155, Cys 165, and Leu 168 of both monomers in the NS5A dimer. Preferably, the amino acid residues that form the interaction site comprise Ser 81, His 85, Leu 110, Glu 152, Gly 155, Cys 165, and Leu 168 of one monomer, and Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of the other monomer. More preferably, the amino acid residues that form the interaction site comprise Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of both monomers. Because the interaction site includes areas from both monomers, a compound of this aspect of the invention often has a dimeric or dimeric-like structure, with one part of the compound fitting to the interaction area provided by the first NS5A monomer and the other part fitting to the interaction area provided by the second NS5A monomer. The two parts of the compound can be the same or contain different moieties. For instance, the compound can have a symmetrical structure and consists of two identical moieties, with one moiety fitting to the interaction area provided by the first NS5A monomer and the other identical moiety fitting to the interaction area provided by the second NS5A. Preferably, each monomer in the NS5A dimer comprises amino acid 37-168 of SEQ ID NO:1, or amino acids 36-198 of SEQ ID NO:1. More preferably, each monomer in the NS5A dimer consists of amino acids 37-168 of SEQ ID NO:1, or amino acids 36-198 of SEQ ID NO:1. In this aspect of the invention, a substantial portion of the compound, or the whole compound, preferably fits to the interaction site. The two monomers in the NS5A dimer preferably have the same amino acid sequence.

In still yet another aspect, at least part of a compound of the present invention fits to an interaction site on an NS5A dimer, wherein the amino acid residues that form the interaction site comprise Phe 37, Ser 38, Cys 39, Pro 58, Ser 81, His 85, Leu 110, Glu 152, Gly 155, Cys 165, and Leu 168 of both monomers in the NS5A dimer. Preferably, the amino acid residues that form the interaction site comprise Phe 37, Ser 38, Cys 39, Gln 40, Pro 58, Ser 81, His 85, Leu 110, Glu 152, Gly 155, Cys 165, and Leu 168 of both monomers. More preferably, the amino acid residues that form the interaction site comprise Phe 37, Ser 38, Cys 39, Arg 41, Gly 42, Pro 58, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of both monomers. Highly preferably, the amino acid residues that form the interaction site comprise Phe 37, Ser 38, Cys 39, Gln 40, Arg 41, Gly 42, Pro 58, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168 of both monomers. Because the interaction site includes areas from both monomers, a compound of this aspect of the invention often has a dimeric or dimeric-like structure and comprises at least three moieties. The first moiety fits to the interaction sites formed by Ser 81, His 85, Leu 110, Glu 152, Gly 155, Cys 165, and Leu 168 (or preferably, by Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168) of the first NS5A monomer, the second moiety fits to the interaction sites formed by Ser 81, His 85, Leu 110, Glu 152, Gly 155, Cys 165, and Leu 168 (or preferably, by Arg 41, Gly 42, Ser 81, Asn 82, His 85, Thr 87, Leu 110, Tyr 118, Thr 151, Glu 152, Gly 155, Val 156, Arg 157, Ala 162, Pro 163, Ala 164, Cys 165, Lys 166, and Leu 168) of the second NS5A monomer, and third moiety fits to the region formed by Phe 37, Ser 38, Cys 39 and Pro 58 (or preferably, by Phe 37, Ser 38, Cys 39, Gln 40 and Pro 58) of both monomers. The first and second moieties can be the same or different. Non-limiting examples of the third moiety include -L-E, as defined hereinabove. Preferably, each monomer in the NS5A dimer comprises amino acid 37-168 of SEQ ID NO:1, or amino acids 36-198 of SEQ ID NO:1. More preferably, each monomer in the NS5A dimer consists of amino acids 37-168 of SEQ ID NO:1, or amino acids 36-198 of SEQ ID NO:1. In this aspect of the invention, a substantial portion of the compound, or the whole compound, preferably fits to the interaction site. The two monomers in the NS5A dimer preferably have the same amino acid sequence.

In yet another aspect, the present invention features compounds characterized by their unique three-dimensional conformations and orientation relative to NS5A dimers. When bound to an NS5A dimer, the compound possesses substituent groups, of which at least one atom or a centroid is contained within the Site of Occupation. The Site of Occupation is defined by locating the center point of a spherical volume together with a defined radius around that center point. The center point of a spherical volume is defined by the point of intersection of vectors of a certain length (loci) emanating from particular alpha carbon atoms of the bound dimeric protein. One of skill in the art will recognize that any of the alpha carbons of the bound protein can be used as points of reference.

The reference points for location of the center point include the alpha carbon atoms of protein residues within the NS5A protein. Protein X-ray crystal structures of NS5A dimers at atomic resolution have shown that while non-binding site insertions, deletions and residue variation can occur between NS5A protein from different strains and subtypes, the binding site backbone is maintained and is spatially equivalent. Thus, numbering of protein residues and reference to their alpha carbons may refer to different residue types, but often refer to identical positions in three-dimensional space. Only residues in SEQ ID NO:1 are used in the definition of the Site of Occupation, but it is understood that the corresponding residues from all other HCV subtypes and strains are encompassed by the present invention.

As used herein, one Site of Occupation is defined as the volume which is of spherical shape and has its center point defines by loci from the group of loci consisting of residues of the NS5A protein. In some cases, the binding site consists of two identical monomers, each of which has 447 amino acid residues (SEQ ID NO:9). As used herein, ordinal numbers 1-447 indicate residues (the N-terminal residue is 1) on one monomer and ordinal numbers 1′-447′ indicate the corresponding residues on the other monomer. The center point of the spherical volume is defined by at least three loci selected from the group of loci consisting of 6.5 to 7.5 Angstroms (Ang) from the alpha carbon of residue 37, 5.0 to 6.0 Ang from the alpha carbon of residue 38, 5.5 to 6.5 Ang from the alpha carbon of residue 39, 4.0 to 5.0 Ang from the alpha carbon of residue 40, 6.0 to 7.0 Ang from the alpha carbon of residue 58, 6.5 to 7.5 Ang from the alpha carbon of residue 37′, 5.0 to 6.0 Ang from the alpha carbon of residue 38′, 5.5 to 6.5 Ang from the alpha carbon of residue 39′, 4.0 to 5.0 Ang from the alpha carbon of residue 40′, and 6.0 to 7.0 Ang from the alpha carbon of residue 58′. The radius of the spherical volume is about 3.0 Ang.

A substituent is considered to be within the Site of Occupation is any atom of that substituent is within the Site of Occupation. An atom is within the site of Occupation if the coordinates for the center of that atom are within the volume defined by the Site of Occupation.

The volume of space defined at the Site of Occupation is defined by spheres of a given radius emanating from the loci given above. It is understood that spheres of lower radius claim smaller volumes, and thus in turn, encompass fewer molecules. Most preferred are radii of 4.90 Angstroms or less, preferred are radii of 3.0 or 3.0 Angstroms or less, less preferred are radii of 1.0 Angstrom or less. An atom of a substituent is within this volume if the coordinates for the center of that atom fall within the volume defined for the Site of Occupation. In addition, a substituent or ring system is within the Site of Occupation if the centroid of the ring atoms is found within the volume. A centroid of a ring is defined as the geometric or arithmetic centerpoint of the coordinates of the atoms that comprise the ring.

In addition, the present invention features compounds modified based on those disclosed in US Patent Application Publication Nos. 20080044379, 20080044380, 20080050336, 20080299075, 20080311075, 20090041716, 20090043107, 20090068140, 20090202478, 20090202483, 20100158862, 20100215616, 20100233120, 20100249190, 20100260708, 20100068176, 20100080772, 20100221214, 20100221215, 20100221216, 20100226882, 20100226883, 20100233122, 20100260715 and 20100266543, and U.S. Pat. Nos. 7,659,270, 7,704,992, 7,728,027, 7,741,347, 7,745,636 and 7,759,495, as well as WO2009003009, WO2010091413, WO2010096462, WO2010099527, WO2010065668, WO2010065668, WO2010065674, WO2010065681, WO2010096777 and WO2010111534, WO2010111673. All of these published US patent applications, US patents and WO publications are incorporated herein by reference in their entireties. Specifically, the NS5A inhibitors disclosed in the above publications can be modified by adding a moiety (e.g., -L-E or -L₃-D, as described above) to the linker regions of these dimer or dimer-like inhibitors so as to allow the modified compounds to interact with the unique binding site(s) identified by the present invention. For example, -L-E or -L₃-D can be added to the linker -D-A-T-E- in Formula I of US 20100221215 or can be substituted with at least one of R¹ or R²; or -L-E or -L₃-D can be added to the linker -Y-A-Z- in Formula I of US 20100226883 or can be substituted with at least one of R¹ or R²; or -L-E or -L₃-D can be added to the linker -L- in Formula I of US 20100226882 or can be substituted with at least one of R¹ or R²; or -L-E or -L₃-D can be added to the linker -L- in Formula I of US 20100266543 or can be substituted with at least one of R¹ or R²; or -L-E or -L₃-D can be added to the linker -A- in Formula 1-I, or the linker -Y-A-Z- in Formulae 2-I or 3-I, of WO2010096462 or can be substituted with at least one of R¹ or R²; or L-E or -L₃-D can be added to the linker -A- in Formulae 1-I or 2-I, or the linker -D-A-T-E- in Formula 3-I, of WO2010091413 or can be substituted with at least one of R¹ or R². Likewise, -L-E or -L₃-D can be added to the linker -B- in the formula of the first aspect in WO2010065681 or WO2010096777. Similarly, -L-E or -L₃-D can be substituted with at least one of R¹ or R² (or R¹, R², R¹′, or R²′,) in the formulae of U.S. Pat. Nos. 7,659,270, 7,704,992, 7,728,027, 7,745,636 and 7,759,49, or added to rings A or B in the formulae of U.S. Pat. Nos. 7,741,347, 7,745,636 and 775,949; or added to the linker -L- in Formula I of US 20100068176.

Furthermore, the present invention features rational drug design of NS5A inhibitors based on the binding sites identified by the present invention. Computational techniques can be used to design compounds capable of binding to NS5A dimers. Knowledge of the structure coordinates for an NS5A dimer permits, for example, the design of compounds that have a shape complementary to the conformation of the interaction sites of the NS5A dimer. In some cases, a compound to be designed is capable of structurally associating with at least one interaction site of the NS5A dimer, and is able, sterically and energetically, to assume a conformation that allows it to associate with the NS5A dimer. Non-covalent molecular interactions important in this association include hydrogen bonding, van der Waals interactions, hydrophobic interactions, electrostatic interactions, or combinations thereof. Conformational considerations include the overall three-dimensional structure and orientation of the compound in relation to the interaction site, and the spacing between various functional groups of the compound that directly interact with the interaction site of the NS5A dimer. There are many compound design methods including, without limitation, LUDI (Bohm, J. COMPUT. AIDED MOLEC. DESIGN 6:61-78 (1992), available from Accelrys, San Diego, Calif.), LEGEND (Nishibata and Itai, J. MED. CHEM. 36:2921-8 (1993), available from Accelrys, San Diego, Calif.), LeapFrog (available from Tripos Associates, St. Louis, Mo.), and SPROUT (Gillet, et al., J. COMPUT. AIDED MOL. DESIGN 7:127-53 (1993), available from the University of Leeds, UK).

Optionally, the potential binding of a compound to an NS5A dimer is analyzed using computer docking programs prior to the actual synthesis and testing of the compound. If these computational experiments suggest sufficient interaction or association between the compound and the NS5A dimer, further testing of the compound is desired. The docking programs described above can be used to screen compounds for their ability to associate with NS5A dimers. In one example, this process may begin by visual inspection of, for example, an interaction site of an NS5A dimer on the computer screen based on the structure coordinates of the dimer. The compound may then be positioned in a variety of orientations, or docked, within the interaction site. Docking can be accomplished using any of the programs described above, such as GRID, MCSS, AUTODOCK, or DOCK, and in one embodiment, QUANTA and SYBYL, followed by energy minimization and molecular dynamics with standard molecular mechanics force fields, such as CHARMM and AMBER.

A compound designed according to the present invention can be further computationally optimized so that in its bound state it would lack repulsive electrostatic interaction with the target protein and/or with the surrounding water or other solvent molecules. Such non-complementary electrostatic interactions include repulsive charge-charge, dipole-dipole, and charge-dipole interactions.

Specific computer software is available to evaluate compound deformation energy and electrostatic interactions. Examples of programs designed for such uses include: Gaussian (M. J. Frisch, Gaussian, Inc., Pittsburgh, Pa.); AMBER (University of California at San Francisco); QUANTA/CHARMM (Accelrys, San Diego, Calif.); Insight II/Discovery (Accelrys, San Diego, Calif.); DelPhi (Accelrys, San Diego, Calif.), and AMSOL (University of Minnesota). These programs can be implemented, for instance, using a Silicon Graphics workstation, such as an Indigo2 with IMPACT graphics. Other hardware systems and software packages will be known to those skilled in the art.

The compounds of the present invention can be used in the form of salts. Depending on the particular compound, a salt of a compound may be advantageous due to one or more of the salt's physical properties, such as enhanced pharmaceutical stability under certain conditions or desired solubility in water or oil. In some instances, a salt of a compound may be useful for the isolation or purification of the compound.

Where a salt is intended to be administered to a patient, the salt preferably is pharmaceutically acceptable. Pharmaceutically acceptable salts include, but are not limited to, acid addition salts, base addition salts, and alkali metal salts.

Pharmaceutically acceptable acid addition salts may be prepared from inorganic or organic acids. Examples of suitable inorganic acids include, but are not limited to, hydrochloric, hydrobromic, hydroionic, nitric, carbonic, sulfuric, and phosphoric acid. Examples of suitable organic acids include, but are not limited to, aliphatic, cycloaliphatic, aromatic, araliphatic, heterocyclyl, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, b-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, hemisulfate, heptanoate, hexanoate, nicotinate, 2-naphthalesulfonate, oxalate, palmoate, pectinate, persulfate, 3-phenylpropionate, picrate, pivalate, thiocyanate, tosylate, and undecanoate.

Pharmaceutically acceptable base addition salts include, but are not limited to, metallic salts and organic salts. Non-limiting examples of suitable metallic salts include alkali metal (group Ia) salts, alkaline earth metal (group IIa) salts, and other pharmaceutically acceptable metal salts. Such salts may be made, without limitation, from aluminum, calcium, lithium, magnesium, potassium, sodium, or zinc. Non-limiting examples of suitable organic salts can be made from tertiary amines and quaternary amine, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as alkyl halides (e.g., methyl, ethyl, propyl, butyl, decyl, lauryl, myristyl, and stearyl chlorides/bromides/iodides), dialkyl sulfates (e.g., dimethyl, diethyl, dibuytl, and diamyl sulfates), aralkyl halides (e.g., benzyl and phenethyl bromides), and others.

The compounds or salts of the present invention may exist in the form of solvates, such as with water (i.e., hydrates), or with organic solvents (e.g., with methanol, ethanol or acetonitrile to form, respectively, methanolate, ethanolate or acetonitrilate).

The compounds or salts of the present invention may also be used in the form of prodrugs. Some prodrugs are aliphatic or aromatic esters derived from acidic groups on the compounds of the invention. Others are aliphatic or aromatic esters of hydroxyl or amino groups on the compounds of the invention. Phosphate prodrugs of hydroxyl groups are preferred prodrugs.

The compounds of the invention may comprise asymmetrically substituted carbon atoms known as chiral centers. These compounds may exist, without limitation, as single stereoisomers (e.g., single enantiomers or single diastereomer), mixtures of stereoisomers (e.g. a mixture of enantiomers or diastereomers), or racemic mixtures. Compounds identified herein as single stereoisomers are meant to describe compounds that are present in a form that is substantially free from other stereoisomers (e.g., substantially free from other enantiomers or diastereomers). By “substantially free,” it means that at least 80% of the compound in a composition is the described stereoisomer; preferably, at least 90% of the compound in a composition is the described stereoisomer; and more preferably, at least 95%, 96%, 97%, 98% or 99% of the compound in a composition is the described stereoisomer. Where the stereochemistry of a chiral carbon is not specified in the chemical structure of a compound, the chemical structure is intended to encompass compounds containing either stereoisomer of the chiral center.

Individual stereoisomers of the compounds of this invention can be prepared using a variety of methods known in the art. These methods include, but are not limited to, stereospecific synthesis, chromatographic separation of diastereomers, chromatographic resolution of enantiomers, conversion of enantiomers in an enantiomeric mixture to diastereomers followed by chromatographically separation of the diastereomers and regeneration of the individual enantiomers, and enzymatic resolution.

Stereospecific synthesis typically involves the use of appropriate optically pure (enantiomerically pure) or substantial optically pure materials and synthetic reactions that do not cause racemization or inversion of stereochemistry at the chiral centers. Mixtures of stereoisomers of compounds, including racemic mixtures, resulting from a synthetic reaction may be separated, for example, by chromatographic techniques as appreciated by those of ordinary skill in the art. Chromatographic resolution of enantiomers can be accomplished by using chiral chromatography resins, many of which are commercially available. In a non-limiting example, racemate is placed in solution and loaded onto the column containing a chiral stationary phase. Enantiomers can then be separated by HPLC.

Resolution of enantiomers can also be accomplished by converting enantiomers in a mixture to diastereomers by reaction with chiral auxiliaries. The resulting diastereomers can be separated by column chromatography or crystallization/re-crystallization. This technique is useful when the compounds to be separated contain a carboxyl, amino or hydroxyl group that will form a salt or covalent bond with the chiral auxiliary. Non-limiting examples of suitable chiral auxiliaries include chirally pure amino acids, organic carboxylic acids or organosulfonic acids. Once the diastereomers are separated by chromatography, the individual enantiomers can be regenerated. Frequently, the chiral auxiliary can be recovered and used again.

Enzymes, such as esterases, phosphatases or lipases, can be useful for the resolution of derivatives of enantiomers in an enantiomeric mixture. For example, an ester derivative of a carboxyl group in the compounds to be separated can be treated with an enzyme which selectively hydrolyzes only one of the enantiomers in the mixture. The resulting enantiomerically pure acid can then be separated from the unhydrolyzed ester.

Alternatively, salts of enantiomers in a mixture can be prepared using any suitable method known in the art, including treatment of the carboxylic acid with a suitable optically pure base such as alkaloids or phenethylamine, followed by precipitation or crystallization/re-crystallization of the enantiomerically pure salts. Methods suitable for the resolution/separation of a mixture of stereoisomers, including racemic mixtures, can be found in ENANTIOMERS, RACEMATES, AND RESOLUTIONS (Jacques et al., 1981, John Wiley and Sons, New York, N.Y.).

A compound of this invention may possess one or more unsaturated carbon-carbon double bonds. All double bond isomers, such as the cis (Z) and trans (E) isomers, and mixtures thereof are intended to be encompassed within the scope of a recited compound unless otherwise specified. In addition, where a compound exists in various tautomeric forms, a recited compound is not limited to any one specific tautomer, but rather is intended to encompass all tautomeric forms.

Certain compounds of the invention may exist in different stable conformational forms which may be separable. Torsional asymmetry due to restricted rotations about an asymmetric single bond, for example because of steric hindrance or ring strain, may permit separation of different conformers. The invention encompasses each conformational isomer of these compounds and mixtures thereof.

Certain compounds of the invention may also exist in zwitterionic form and the invention encompasses each zwitterionic form of these compounds and mixtures thereof.

The compounds of the present invention are generally described herein using standard nomenclature. For a recited compound having asymmetric center(s), it should be understood that all of the stereoisomers of the compound and mixtures thereof are encompassed in the present invention unless otherwise specified. Non-limiting examples of stereoisomers include enantiomers, diastereomers, and cis-transisomers. Where a recited compound exists in various tautomeric forms, the compound is intended to encompass all tautomeric forms. Certain compounds are described herein using general formulas that include variables (e.g., R_{A} or R_{B}). Unless otherwise specified, each variable within such a formula is defined independently of any other variable, and any variable that occurs more than one time in a formula is defined independently at each occurrence. If moieties are described as being “independently” selected from a group, each moiety is selected independently from the other. Each moiety therefore can be identical to or different from the other moiety or moieties.

The number of carbon atoms in a hydrocarbyl moiety can be indicated by the prefix “Cₓ—C_{y},” where x is the minimum and y is the maximum number of carbon atoms in the moiety. Thus, for example, “C₁-C₆alkyl” refers to an alkyl substituent containing from 1 to 6 carbon atoms. Illustrating further, C₃-C₆carbocycle means a carbocycle containing from 3 to 6 carbon ring atoms. A prefix attached to a multiple-component substituent only applies to the first component that immediately follows the prefix. To illustrate, the term “carbocyclylalkyl” contains two components: carbocyclyl and alkyl. Thus, for example, C₃-C₆carbocyclylC₁-C₆alkyl refers to a C₃-C₆-carbocyclyl appended to the parent molecular moiety through a C₁-C₆alkyl group.

Unless otherwise specified, when a linking element links two other elements in a depicted chemical structure, the leftmost-described component of the linking element is bound to the left element in the depicted structure, and the rightmost-described component of the linking element is bound to the right element in the depicted structure. To illustrate, if the chemical structure is -L-L_{S}-R_{E} and L_{S} is C₁-C₆alkylene, then the chemical structure is -L-C₁-C₆alkylene-R_{E}.

If a linking element in a depicted structure is a bond, then the element left to the linking element is joined directly to the element right to the linking element via a covalent bond. For example, if a chemical structure is depicted as -L-L_{S}-R_{E} and L_{S} is selected as bond, then the chemical structure will be -L-R_{E}. If two or more adjacent linking elements in a depicted structure are bonds, then the element left to these linking elements is joined directly to the element right to these linking elements via a covalent bond.

When a chemical formula is used to describe a moiety, the dash(s) indicates the portion of the moiety that has the free valence(s).

If a moiety is described as being “optionally substituted”, the moiety may be either substituted or unsubstituted. If a moiety is described as being optionally substituted with up to a particular number of non-hydrogen radicals, that moiety may be either unsubstituted, or substituted by up to that particular number of non-hydrogen radicals or by up to the maximum number of substitutable positions on the moiety, whichever is less. Thus, for example, if a moiety is described as a heterocycle optionally substituted with up to three non-hydrogen radicals, then any heterocycle with less than three substitutable positions will be optionally substituted by up to only as many non-hydrogen radicals as the heterocycle has substitutable positions. To illustrate, tetrazolyl (which has only one substitutable position) will be optionally substituted with up to one non-hydrogen radical. To illustrate further, if an amino nitrogen is described as being optionally substituted with up to two non-hydrogen radicals, then a primary amino nitrogen will be optionally substituted with up to two non-hydrogen radicals, whereas a secondary amino nitrogen will be optionally substituted with up to only one non-hydrogen radical.

The term “alkenyl” means a straight or branched hydrocarbyl chain containing one or more double bonds. Each carbon-carbon double bond may have either cis or trans geometry within the alkenyl moiety, relative to groups substituted on the double bond carbons. Non-limiting examples of alkenyl groups include ethenyl (vinyl), 2-propenyl, 3-propenyl, 1,4-pentadienyl, 1,4-butadienyl, 1-butenyl, 2-butenyl, and 3-butenyl.

The term “alkenylene” refers to a divalent unsaturated hydrocarbyl chain which may be linear or branched and which has at least one carbon-carbon double bond. Non-limiting examples of alkenylene groups include —C(H)═C(H)—, —C(H)═C(H)—CH₂—, —C(H)═C(H)—CH₂—CH₂—, —CH₂—C(H)═C(H)—CH₂—, —C(H)═C(H)—CH(CH₃)—, and —CH₂—C(H)═C(H)—CH(CH₂CH₃)—.

The term “alkyl” means a straight or branched saturated hydrocarbyl chain. Non-limiting examples of alkyl groups include methyl, ethyl, n-propyl, isopropyl, n-butyl, isobutyl, sec-butyl, t-butyl, pentyl, iso-amyl, and hexyl.

The term “alkylene” denotes a divalent saturated hydrocarbyl chain which may be linear or branched. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkynyl” means a straight or branched hydrocarbyl chain containing one or more triple bonds. Non-limiting examples of alkynyl include ethynyl, 1-propynyl, 2-propynyl, 3-propynyl, decynyl, 1-butynyl, 2-butynyl, and 3-butynyl.

The term “alkynylene” refers to a divalent unsaturated hydrocarbon group which may be linear or branched and which has at least one carbon-carbon triple bonds. Representative alkynylene groups include, by way of example, —C≡C—, —C≡C—CH₂—, —C≡C—CH₂—CH₂—, —CH₂—C≡C—CH₂—, —C≡C—CH(CH₃)—, and —CH₂—C≡C—CH(CH₂CH₃)—.

The term “carbocycle” or “carbocyclic” or “carbocyclyl” refers to a saturated (e.g., “cycloalkyl”), partially saturated (e.g., “cycloalkenyl” or “cycloalkynyl”) or completely unsaturated (e.g., “aryl”) ring system containing zero heteroatom ring atom. “Ring atoms” or “ring members” are the atoms bound together to form the ring or rings. A carbocyclyl may be, without limitation, a single ring, two fused rings, or bridged or spiro rings. A substituted carbocyclyl may have either cis or trans geometry. Representative examples of carbocyclyl groups include, but are not limited to, cyclopropyl, cyclobutyl, cyclopentyl, cyclohexyl, cycloheptyl, cyclooctyl, cyclopentenyl, cyclopentadienyl, cyclohexadienyl, adamantyl, decahydro-naphthalenyl, octahydro-indenyl, cyclohexenyl, phenyl, naphthyl, indanyl, 1,2,3,4-tetrahydro-naphthyl, indenyl, isoindenyl, decalinyl, and norpinanyl. A carbocycle group can be attached to the parent molecular moiety through any substitutable carbon ring atom.

The term “carbocyclylalkyl” refers to a carbocyclyl group appended to the parent molecular moiety through an alkylene group. For instance, C₃-C₆carbocyclylC₁-C₆alkyl refers to a C₃-C₆carbocyclyl group appended to the parent molecular moiety through C₁-C₆alkylene.

The term “cycloalkenyl” refers to a non-aromatic, partially unsaturated carbocyclyl moiety having zero heteroatom ring member. Representative examples of cycloalkenyl groups include, but are not limited to, cyclobutenyl, cyclopentenyl, cyclohexenyl, and octahydronaphthalenyl.

The term “cycloalkyl” refers to a saturated carbocyclyl group containing zero heteroatom ring member. Non-limiting examples of cycloalkyls include cyclopropyl, cyclobutyl, cyclopentyl, cyclohexyl, cycloheptyl, cyclooctyl, decalinyl and norpinanyl.

The prefix “halo” indicates that the substituent to which the prefix is attached is substituted with one or more independently selected halogen radicals. For example, “C₁-C₆haloalkyl” means a C₁-C₆alkyl substituent wherein one or more hydrogen atoms are replaced with independently selected halogen radicals. Non-limiting examples of C₁-C₆haloalkyl include chloromethyl, 1-bromoethyl, fluoromethyl, difluoromethyl, trifluoromethyl, and 1,1,1-trifluoroethyl. It should be recognized that if a substituent is substituted by more than one halogen radical, those halogen radicals may be identical or different (unless otherwise stated).

The term “heterocycle” or “heterocyclo” or “heterocyclyl” refers to a saturated (e.g., “heterocyclo alkyl”), partially unsaturated (e.g., “heterocycloalkenyl” or “heterocycloalkynyl”) or completely unsaturated (e.g., “heteroaryl”) ring system where at least one of the ring atoms is a heteroatom (i.e., nitrogen, oxygen or sulfur), with the remaining ring atoms being independently selected from the group consisting of carbon, nitrogen, oxygen and sulfur. A heterocycle may be, without limitation, a single ring, two fused rings, or bridged or spiro rings. A heterocycle group can be linked to the parent molecular moiety via any substitutable carbon or nitrogen atom(s) in the group.

A heterocyclyl may be, without limitation, a monocycle which contains a single ring. Non-limiting examples of monocycles include furanyl, dihydrofuranyl, tetrahydrofuranyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiodiazolyl, oxathiazolyl, oxadiazolyl (including 1,2,3-oxadiazolyl, 1,2,4-oxadiazolyl (also known as “azoximyl”), 1,2,5-oxadiazolyl (also known as “furazanyl”), and 1,3,4-oxadiazolyl), oxatriazolyl (including 1,2,3,4-oxatriazolyl and 1,2,3,5-oxatriazolyl), dioxazolyl (including 1,2,3-dioxazolyl, 1,2,4-dioxazolyl, 1,3,2-dioxazolyl, and 1,3,4-dioxazolyl), oxathiolanyl, pyranyl (including 1,2-pyranyl and 1,4-pyranyl), dihydropyranyl, pyridinyl, piperidinyl, diazinyl (including pyridazinyl (also known as “1,2-diazinyl”), pyrimidinyl (also known as “1,3-diazinyl”), and pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl), oxazinyl (including 1,2,3-oxazinyl, 1,3,2-oxazinyl, 1,3,6-oxazinyl (also known as “pentoxazolyl”), 1,2,6-oxazinyl, and 1,4-oxazinyl), isoxazinyl (including o-isoxazinyl and p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl and 1,3,5,2-oxadiazinyl), morpholinyl, azepinyl, oxepinyl, thiepinyl, and diazepinyl.

A heterocyclyl may also be, without limitation, a bicycle containing two fused rings, such as, for example, naphthyridinyl (including [1,8]naphthyridinyl, and [1,6]naphthyridinyl), thiazolpyrimidinyl, thienopyrimidinyl, pyrimidopyrimidinyl, pyridopyrimidinyl, pyrazolopyrimidinyl, indolizinyl, pyrindinyl, pyranopyrrolyl, 4H-quinolizinyl, purinyl, pyridopyridinyl (including pyrido[3,4-b]-pyridinyl, pyrido[3,2-b]-pyridinyl, and pyrido[4,3-b]-pyridinyl), pyridopyrimidine, and pteridinyl. Other non-limiting examples of fused-ring heterocycles include benzo-fused heterocyclyls, such as indolyl, isoindolyl, indoleninyl (also known as “pseudoindolyl”), isoindazolyl (also known as “benzpyrazolyl”), benzazinyl (including quinolinyl (also known as “1-benzazinyl”) and isoquinolinyl (also known as “2-benzazinyl”)), benzimidazolyl, phthalazinyl, quinoxalinyl, benzodiazinyl (including cinnolinyl (also known as “1,2-benzodiazinyl”) and quinazolinyl (also known as “1,3-benzodiazinyl”)), benzopyranyl (including “chromenyl” and “isochromenyl”), benzothiopyranyl (also known as “thiochromenyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl”, “thionaphthenyl”, and “benzothiofuranyl”), isobenzothienyl (also known as “isobenzothiophenyl”, “isothionaphthenyl”, and “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, and 3,1,4-benzoxazinyl), benzisoxazinyl (including 1,2-benzisoxazinyl and 1,4-benzisoxazinyl), and tetrahydroisoquinolinyl.

A heterocyclyl may comprise one or more sulfur atoms as ring members; and in some cases, the sulfur atom(s) is oxidized to SO or SO₂. The nitrogen heteroatom(s) in a heterocyclyl may or may not be quaternized, and may or may not be oxidized to N-oxide. In addition, the nitrogen heteroatom(s) may or may not be N-protected.

The term “pharmaceutically acceptable” is used adjectivally to mean that the modified noun is appropriate for use as a pharmaceutical product or as a part of a pharmaceutical product.

The term “therapeutically effective amount” refers to the total amount of each active substance that is sufficient to show a meaningful patient benefit, e.g. a reduction in viral load.

The term “prodrug” refers to derivatives of the compounds of the invention which have chemically or metabolically cleavable groups and become, by solvolysis or under physiological conditions, the compounds of the invention which are pharmaceutically active in vivo. A prodrug of a compound may be formed in a conventional manner by reaction of a functional group of the compound (such as an amino, hydroxy or carboxy group). Prodrugs often offer advantages of solubility, tissue compatibility, or delayed release in mammals (see, Bungard, H., DESIGN OF PRODRUGS, pp. 7-9, 21-24, Elsevier, Amsterdam 1985). Prodrugs include acid derivatives well known to practitioners of the art, such as, for example, esters prepared by reaction of the parent acidic compound with a suitable alcohol, or amides prepared by reaction of the parent acid compound with a suitable amine Examples of prodrugs include, but are not limited to, acetate, formate, benzoate or other acylated derivatives of alcohol or amine functional groups within the compounds of the invention.

The term “solvate” refers to the physical association of a compound of this invention with one or more solvent molecules, whether organic or inorganic. This physical association often includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include, but are not limited to, hydrates, ethanolates, and methanolates.

The term “N-protecting group” or “N-protected” refers to those groups capable of protecting an amino group against undesirable reactions. Commonly used N-protecting groups are described in Greene and Wuts, PROTECTING GROUPS IN CHEMICAL SYNTHESIS (3^{rd} ed., John Wiley & Sons, NY (1999). Non-limiting examples of N-protecting groups include acyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, benzoyl, 4-chlorobenzoyl, 4-bromobenzoyl, or 4-nitrobenzoyl; sulfonyl groups such as benzenesulfonyl or p-toluenesulfonyl; sulfenyl groups such as phenylsulfenyl (phenyl-S—) or triphenylmethylsulfenyl (trityl-S—); sulfinyl groups such as p-methylphenylsulfinyl (p-methylphenyl-S(O)—) or t-butylsulfinyl (t-Bu-S(O)—); carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, trimethoxybenzyloxycarbonyl, 1-(p-biphenylyl)-1-methylethoxycarbonyl, dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2-trichloro-ethoxy-carbonyl, phenoxycarbonyl, 4-nitro-phenoxycarbonyl, cyclopentyloxycarbonyl, adamantyloxycarbonyl, cyclohexyloxycarbonyl, or phenylthiocarbonyl; alkyl groups such as benzyl, p-methoxybenzyl, triphenylmethyl, or benzyloxymethyl; p-methoxyphenyl; and silyl groups such as trimethylsilyl. Preferred N-protecting groups include formyl, acetyl, benzoyl, pivaloyl, t-butylacetyl, phenylsulfonyl, benzyl, t-butyloxycarbonyl (Boc) and benzyloxycarbonyl (Cbz).

The present invention also features pharmaceutical compositions comprising the compounds of the invention. A pharmaceutical composition of the present invention can comprise one or more compounds of the invention.

In addition, the present invention features pharmaceutical compositions comprising pharmaceutically acceptable salts, solvates, or prodrugs of the compounds of the invention. Without limitation, pharmaceutically acceptable salts can be zwitterions or derived from pharmaceutically acceptable inorganic or organic acids or bases. Preferably, a pharmaceutically acceptable salt retains the biological effectiveness of the free acid or base of the compound without undue toxicity, irritation, or allergic response, has a reasonable benefit/risk ratio, is effective for the intended use, and is not biologically or otherwise undesirable.

The present invention further features pharmaceutical compositions comprising a compound of the invention (or a salt, solvate or prodrug thereof) and another therapeutic agent. By way of illustration not limitation, these other therapeutic agents can be selected from antiviral agents (e.g., anti-HIV agents, anti-HBV agents, or other anti-HCV agents such as HCV protease inhibitors, HCV polymerase inhibitors, HCV helicase inhibitors, IRES inhibitors or NS5A inhibitors), anti-bacterial agents, anti-fungal agents, immunomodulators, anti-cancer or chemotherapeutic agents, anti-inflammation agents, antisense RNA, siRNA, antibodies, or agents for treating cirrhosis or inflammation of the liver. Specific examples of these other therapeutic agents include, but are not limited to, ribavirin, α-interferon, β-interferon, pegylated interferon-α, pegylated interferon-lambda, ribavirin, viramidine, R-5158, nitazoxanide, amantadine, Debio-025, NIM-811, R7128, R1626, R4048, T-1106, PSI-7851, PF-00868554, ANA-598, IDX184, IDX102, IDX375, GS-9190, VCH-759, VCH-916, MK-3281, BCX-4678, MK-3281, VBY708, ANA598, GL59728, GL60667, BMS-790052, BMS-791325, BMS-650032, GS-9132, ACH-1095, AP-H005, A-831, A-689, AZD2836, telaprevir, boceprevir, ITMN-191, BI-201335, VBY-376, VX-500 (Vertex), PHX-B, ACH-1625, IDX136, IDX316, VX-813 (Vertex), SCH 900518 (Schering-Plough), TMC-435 (Tibotec), ITMN-191 (Intermune, Roche), MK-7009 (Merck), IDX-PI (Novartis), BI-201335 (Boehringer Ingelheim), R7128 (Roche), PSI-7851 (Pharmasset), MK-3281 (Merck), PF-868554 (Pfizer), IDX-184 (Novartis), IDX-375 (Pharmasset), BILB-1941 (Boehringer Ingelheim), GS-9190 (Gilead), BMS-790052 (BMS), Albuferon (Novartis), ritonavir, another cytochrome P450 monooxygenase inhibitor, or any combination thereof.

In one embodiment, a pharmaceutical composition of the present invention comprises one or more compounds of the present invention (or salts, solvates or prodrugs thereof), and one or more other antiviral agents.

In another embodiment, a pharmaceutical composition of the present invention comprises one or more compounds of the present invention (or salts, solvates or prodrugs thereof), and one or more other anti-HCV agents, such as an agent selected from HCV polymerase inhibitors (including nucleoside or non-nucleoside type of polymerase inhibitors), HCV protease inhibitors, HCV helicase inhibitors, CD81 inhibitors, cyclophilin inhibitors, IRES inhibitors, or NS5A inhibitors.

In yet another embodiment, a pharmaceutical composition of the present invention comprises one or more compounds of the present invention (or salts, solvates or prodrugs thereof), and one or more other antiviral agents, such as anti-HBV, anti-HIV agents, or anti-hepatitis A, anti-hepatitis D, anti-hepatitis E or anti-hepatitis G agents. Non-limiting examples of anti-HBV agents include adefovir, lamivudine, and tenofovir. Non-limiting examples of anti-HIV drugs include ritonavir, lopinavir, indinavir, nelfinavir, saquinavir, amprenavir, atazanavir, tipranavir, TMC-114, fosamprenavir, zidovudine, lamivudine, didanosine, stavudine, tenofovir, zalcitabine, abacavir, efavirenz, nevirapine, delavirdine, TMC-125, L-870812, S-1360, enfuvirtide, T-1249, or other HIV protease, reverse transcriptase, integrase or fusion inhibitors. Any other desirable antiviral agents can also be included in a pharmaceutical composition of the present invention, as appreciated by those skilled in the art.

A pharmaceutical composition of the present invention typically includes a pharmaceutically acceptable carrier or excipient. Non-limiting examples of suitable pharmaceutically acceptable carriers/excipients include sugars (e.g., lactose, glucose or sucrose), starches (e.g., corn starch or potato starch), cellulose or its derivatives (e.g., sodium carboxymethyl cellulose, ethyl cellulose or cellulose acetate), oils (e.g., peanut oil, cottonseed oil, safflower oil, sesame oil, olive oil, corn oil or soybean oil), glycols (e.g., propylene glycol), buffering agents (e.g., magnesium hydroxide or aluminum hydroxide), agar, alginic acid, powdered tragacanth, malt, gelatin, talc, cocoa butter, pyrogen-free water, isotonic saline, Ringer's solution, ethanol, or phosphate buffer solutions. Lubricants, coloring agents, releasing agents, coating agents, sweetening, flavoring or perfuming agents, preservatives, or antioxidants can also be included in a pharmaceutical composition of the present invention.

The pharmaceutical compositions of the present invention can be formulated based on their routes of administration using methods well known in the art. For example, a sterile injectable preparation can be prepared as a sterile injectable aqueous or oleagenous suspension using suitable dispersing or wetting agents and suspending agents. Suppositories for rectal administration can be prepared by mixing drugs with a suitable nonirritating excipient such as cocoa butter or polyethylene glycols which are solid at ordinary temperatures but liquid at the rectal temperature and will therefore melt in the rectum and release the drugs. Solid dosage forms for oral administration can be capsules, tablets, pills, powders or granules. In such solid dosage forms, the active compounds can be admixed with at least one inert diluent such as sucrose lactose or starch. Solid dosage forms may also comprise other substances in addition to inert diluents, such as lubricating agents. In the case of capsules, tablets and pills, the dosage forms may also comprise buffering agents. Tablets and pills can additionally be prepared with enteric coatings. Liquid dosage forms for oral administration can include pharmaceutically acceptable emulsions, solutions, suspensions, syrups or elixirs containing inert diluents commonly used in the art. Liquid dosage forms may also comprise wetting, emulsifying, suspending, sweetening, flavoring, or perfuming agents. The pharmaceutical compositions of the present invention can also be administered in the form of liposomes, as described in U.S. Pat. No. 6,703,403. Formulation of drugs that are applicable to the present invention is generally discussed in, for example, Hoover, John E., REMINGTON′S PHARMACEUTICAL SCIENCES (Mack Publishing Co., Easton, Pa.: 1975), and Lachman, L., eds., PHARMACEUTICAL DOSAGE FORMS (Marcel Decker, New York, N.Y., 1980).

Any compound described herein, or a pharmaceutically acceptable salt thereof, can be used to prepared pharmaceutical compositions of the present invention.

The present invention further features methods of using the compounds of the present invention (or salts, solvates or prodrugs thereof) to inhibit HCV replication. The methods comprise contacting cells infected with HCV virus with an effective amount of a compound of the present invention (or a salt, solvate or prodrug thereof), thereby inhibiting the replication of HCV virus in the cells. As used herein, “inhibiting” means significantly reducing, or abolishing, the activity being inhibited (e.g., viral replication). In many cases, representative compounds of the present invention can reduce the replication of HCV virus (e.g., in an HCV replicon assay as described above) by at least 10%, 20%, 30%, 40%, 50%, 60%, 70%, 80%, 90%, 95% or more.

The compounds of the present invention may inhibit one or more HCV subtypes. Examples of HCV subtypes that are amenable to the present invention include, but are not be limited to, HCV genotypes 1, 2, 3, 4, 5 and 6, including HCV genotypes 1a, 1b, 2a, 2b, 2c or 3a. In one embodiment, a compound or compounds of the present invention (or salts, solvates or prodrugs thereof) are used to inhibit the replication of HCV genotype 1a. In another embodiment, a compound or compounds of the present invention (or salts, solvates or prodrugs thereof) are used to inhibit the replication of HCV genotype 1b. In still another embodiment, a compound or compounds of the present invention (or salts, solvates or prodrugs thereof) are used to inhibit the replication of both HCV genotypes 1a and 1b.

The present invention also features methods of using the compounds of the present invention (or salts, solvates or prodrugs thereof) to treat HCV infection. The methods typically comprise administering a therapeutic effective amount of a compound of the present invention (or a salt, solvate or prodrug thereof), or a pharmaceutical composition comprising the same, to an HCV patient, thereby reducing the HCV viral level in the blood or liver of the patient. As used herein, the term “treating” refers to reversing, alleviating, inhibiting the progress of, or preventing the disorder or condition, or one or more symptoms of such disorder or condition to which such term applies. The term “treatment” refers to the act of treating. In one embodiment, the methods comprise administering a therapeutic effective amount of two or more compounds of the present invention (or salts, solvates or prodrugs thereof), or a pharmaceutical composition comprising the same, to an HCV patient, thereby reducing the HCV viral level in the blood or liver of the patient.

A compound of the present invention (or a salt, solvate or prodrug thereof) can be administered as the sole active pharmaceutical agent, or in combination with another desired drug, such as other anti-HCV agents, anti-HIV agents, anti-HBV agents, anti-hepatitis A agents, anti-hepatitis D agents, anti-hepatitis E agents, anti-hepatitis G agents, or other antiviral drugs. Any compound described herein, or a pharmaceutically acceptable salt thereof, can be employed in the methods of the present invention.

A compound of the present invention (or a salt, solvent or prodrug thereof) can be administered to a patient in a single dose or divided doses. A typical daily dosage can range, without limitation, from 0.1 to 200 mg/kg body weight, such as from 0.25 to 100 mg/kg body weight. Single dose compositions can contain these amounts or submultiples thereof to make up the daily dose. Preferably, each dosage contains a sufficient amount of a compound of the present invention that is effective in reducing the HCV viral load in the blood or liver of the patient. The amount of the active ingredient, or the active ingredients that are combined, to produce a single dosage form may vary depending upon the host treated and the particular mode of administration. It will be understood that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy.

The present invention further features methods of using the pharmaceutical compositions of the present invention to treat HCV infection. The methods typically comprise administering a pharmaceutical composition of the present invention to an HCV patient, thereby reducing the HCV viral level in the blood or liver of the patient. Any pharmaceutical composition described herein can be used in the methods of the present invention.

In addition, the present invention features use of the compounds or salts of the present invention for the manufacture of medicaments for the treatment of HCV infection. Any compound described herein, or a pharmaceutically acceptable salt thereof, can be used to make medicaments of the present invention.

The compounds of the present invention can also be isotopically substituted. Preferred isotopic substitution include substitutions with stable or nonradioactive isotopes such as deuterium, ¹³C, ¹⁵N or ¹⁸O. Incorporation of a heavy atom, such as substitution of deuterium for hydrogen, can give rise to an isotope effect that could alter the pharmacokinetics of the drug. In one example, at least 10 mol % of hydrogen in a compound of the present invention is substituted with deuterium. In another example, at least 25 mole % of hydrogen in a compound of the present invention is substituted with deuterium. In a further example, at least 50, 60, 70, 80 or 90 mole % of hydrogen in a compound of the present invention is substituted with deuterium. The natural abundance of deuterium is about 0.015%. Deuterium substitution or enrichment can be achieved, without limitation, by either exchanging protons with deuterium or by synthesizing the molecule with enriched or substituted starting materials. Other methods known in the art can also be used for isotopic substitutions.

The compounds of the present invention can also be isotopically substituted. Preferred isotopic substitution include substitutions with stable or nonradioactive isotopes such as deuterium, ¹³C, ¹⁵N or ¹⁸O. Incorporation of a heavy atom, such as substitution of deuterium for hydrogen, can give rise to an isotope effect that could alter the pharmacokinetics of the drug. In one example, at least 10 mol % of hydrogen in a compound of the present invention is substituted with deuterium. In another example, at least 25 mole % of hydrogen in a compound of the present invention is substituted with deuterium. In a further example, at least 50, 60, 70, 80 or 90 mole % of hydrogen in a compound of the present invention is substituted with deuterium. The natural abundance of deuterium is about 0.015%. Deuterium substitution or enrichment can be achieved, without limitation, by either exchanging protons with deuterium or by synthesizing the molecule with enriched or substituted starting materials. Other methods known in the art can also be used for isotopic substitutions.

The present invention also features machine-readable storage medium that is capable of displaying a graphical three-dimensional representation of the structures of an NS5A dimer and a compound that is docked to the NS5A dimer. The three-dimensional structures and the coordinates of the NS5A dimer and the docked compound can be encoded as data in the data storage medium. The data can be used to computationally evaluate the interaction between the NS5A dimer and the compound.

Furthermore, the present invention also features methods of transmitting or receiving data that encode the structure information of a compound designed or identified according to the present invention. Preferably, the transmission is carried out by electronic means, such as via email or downloading from a local or remote system. Non-limiting examples of a remote system from which a download may be performed include webservers, FTP servers, email servers, or other similar systems. The transmission can also be carried out manually, such as by recording the data in a data storage medium such as a compact disc or a flash memory data storage device. In one embodiment, the present invention provides a method of receiving data which comprise structural information of a compound designed or identified according to the present invention, wherein the method comprises retrieving data from a server or a data storage medium that encodes the structural information of the compound.

The contents of all references (including literature references, issued patents, published patent applications, and co-pending patent applications) cited throughout this application are hereby expressly incorporated herein in their entireties by reference.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A method of identifying NS5A inhibitors, comprising docking a compound to an NS5A dimer using a computer docking program, wherein the NS5A dimer comprises two NS5A monomers and an interaction site, and the amino acid residues that form the interaction site comprise amino acids 37, 38, 39 and 58 of both said monomers, and wherein whether said compound comprises a moiety that fits to the interaction site is indicative of whether said compound is an NS5A inhibitor.

2. The method of claim 1, wherein the amino acid residues that form the interaction site comprise amino acids 37, 38, 39, 40 and 58 of both said monomers.

3. The method of claim 2, wherein each said monomer comprises amino acids 36-198 of SEQ ID NO: 1, 2, 3, 4, 5, 6 or 8 or amino acids 36-197 of SEQ ID NO:7.

4. The method of claim 2, wherein each said monomer comprises amino acids 36-198 of SEQ ID NO: 1.

5. The method of claim 2, wherein the amino acid residues that form the interaction site comprise Phe 37, Ser 38, Cys 39, Gln 40 and Pro 58 of both said monomers

6. A method of identifying NS5A inhibitors, comprising docking a compound to an NS5A dimer using a computer docking program, wherein the NS5A dimer comprises two NS5A monomers and an interaction site, and the amino acid residues that form the interaction site comprise amino acids 81, 85, 110, 152, 155, 165, and 168 of one said monomer, and wherein whether at least part of said compound fits to the interaction site is indicative of whether said compound is an NS5A inhibitor.

7. The method of claim 6, wherein the amino acid residues that form the interaction site comprise amino acids 41, 42, 81, 82, 85, 87, 110, 118, 151, 152, 155, 156, 157, 162, 163, 164, 165, 166 and 168 of one said monomer.

8. The method of claim 7, wherein each said monomer comprises amino acids 36-198 of SEQ ID NO: 1.

9. A method of identifying NS5A inhibitors, comprising docking a compound to an NS5A dimer using a computer docking program, wherein the NS5A dimer comprises two NS5A monomers and an interaction site, and the amino acid residues that form the interaction site comprise amino acids 81, 85, 110, 152, 155, 165, and 168 of both said monomers, and wherein whether at least part of said compound fits to the interaction site is indicative of whether said compound is an NS5A inhibitor.

10. The method of claim 9, wherein the amino acid residues that form the interaction site comprise amino acids 41, 42, 81, 82, 85, 87, 110, 118, 151, 152, 155, 156, 157, 162, 163, 164, 165, 166 and 168 of both said monomers.

11. The method of claim 10, wherein each said monomer comprises amino acids 36-198 of SEQ ID NO: 1.

12. A method of identifying NS5A inhibitors, comprising docking a compound to an NS5A dimer using a computer docking program, wherein the NS5A dimer comprises two NS5A monomers and an interaction site, and the amino acid residues that form the interaction site comprise amino acids 81, 85, 110, 152, 155, 165, and 168 of one said monomer, and amino acids 37, 38, 39, and 58 of both said monomers, and wherein whether at least part of said compound fits to the interaction site is indicative of whether said compound is an NS5A inhibitor.

13. The method of claim 12, wherein the amino acid residues that form the interaction site comprise amino acids 41, 42, 81, 82, 85, 87, 110, 118, 151, 152, 155, 156, 157, 162, 163, 164, 165, 166 and 168 of one said monomer, and amino acids 37, 38, 39, 40 and 58 of both said monomers.

14. The method of claim 13, wherein each said monomer comprises amino acids 36-198 of SEQ ID NO: 1.

15. A method of identifying NS5A inhibitors, comprising docking a compound to an NS5A dimer using a computer docking program, wherein the NS5A dimer comprises two NS5A monomers and an interaction site, and the amino acid residues that form the interaction site comprise amino acids 37, 38, 39, 58, 81, 85, 110, 152, 155, 165, and 168 of both said monomers, and wherein whether at least part of said compound fits to the interaction site is indicative of whether said compound is an NS5A inhibitor.

16. The method of claim 15, wherein the amino acid residues that form the interaction site comprise amino acids 37, 38, 39, 40, 41, 42, 58, 81, 82, 85, 87, 110, 118, 151, 152, 155, 156, 157, 162, 163, 164, 165, 166, and 168 of both said monomers.

17. The method of claim 16, wherein each said monomer comprises amino acids 36-198 of SEQ ID NO: 1.

18. An NS5A inhibitor identified according to a method of claim 1, 12 or 15.

19. An NS5A inhibitor which is capable of being docked to an NS5A dimer using a computer docking program, wherein the NS5A dimer comprises two NS5A monomers and an interaction site, and the amino acid residues that form the interaction site comprise amino acids 37, 38, 39 and 58 of both said monomers, and wherein said compound comprises a moiety that fits to the interaction site.

20. The NS5A inhibitor of claim 19, wherein the amino acid residues that form the interaction site comprise amino acids 37, 38, 39, 40 and 58 of both said monomers.

21. The NS5A inhibitor of claim 20, wherein each said monomer comprises amino acids 36-198 of SEQ ID NO: 1, 2, 3, 4, 5, 6 or 8 or amino acids 36-197 of SEQ ID NO:7.

22. The NS5A inhibitor of claim 20, wherein each said monomer comprises amino acids 36-198 of SEQ ID NO: 1.

23. The NS5A inhibitor of claim 22, wherein said moiety comprises -L-E, and wherein:
E is C₃-C₁₄carbocycle or 3- to 14-membered heterocycle, and is optionally substituted with one or more R_{A}; or E is -L_{S}-R_{E};
L is -L_{S}-, -L_{S}-O-L_{S}′-, -L_{S}-C(O)-L_{S}′-, -L_{S}-S(O)₂-L_{S}′-, -L_{S}-S(O)-L_{S}′-, -L_{S}-OS(O)₂-L_{S}′-, -L_{S}-S(O)₂O-L_{S}′-, -L_{S}-OS(O)-L_{S}′-, -L_{S}-S(O)O-L_{S}′-, -L_{S}-C(O)O-L_{S}′-, -L_{S}-OC(O)-L_{S}′-, -L_{S}-OC(O)O-L_{S}′-, -L_{S}-C(O)N(R_{B})-L_{S}′-, L_{S}-N(R_{B})C(O)-L_{S}′-, -L_{S}-C(O)N(R_{B})O-L_{S}′-, -L_{S}-N(R_{B})C(O)O-L_{S}′-, -L_{S}-OC(O)N(R_{B})-L_{S}′-, -L_{S}-C(O)N(R_{B})N(R_{B}′)-L_{S}′-, -L_{S}-S-L_{S}′-, -L_{S}-C(S)-L_{S}′-, -L_{S}-C(S)O-L_{S}′-, -L_{S}-OC(S)-L_{S}′-, -L_{S}-C(S)N(R_{B})-L_{S}′-, -L_{S}-N(R_{B})-L_{S}′-, -L_{S}-N(R_{B})C(S)-L_{S}′-, -L_{S}-N(R_{B})S(O)-L_{S}′-, L_{S}-N(R_{B})S(O)₂-L_{S}′-, -L_{S}-S(O)₂N(R_{B})-L_{S}′-, -L_{S}-S(O)N(R_{B})-L_{S}′-, -L_{S}-C(S)N(R_{B})O-L_{S}′-, -L_{S}-C(O)N(R_{B})C(O)-L_{S}′-, -L_{S}-N(R_{B})C(O)N(R_{B}′)-L_{S}′-, -L_{S}-N(R_{B})SO₂N(R_{B}′)-L_{S}′-, -L_{S}-N(R_{B})S(O)N(R_{B}′)-L_{S}′-, or -L_{S}-C(S)N(R_{B})N(R_{B}′)-L_{S}′-;
L_{S} and L_{S}′ are each independently selected at each occurrence from bond; or C₁-C₆alkylene, C₂-C₆alkenylene or C₂-C₆alkynylene, each of which is independently optionally substituted at each occurrence with one or more R_{L};
R_{A} is independently selected at each occurrence from halogen, oxo, thioxo, hydroxy, mercapto, nitro, cyano, amino, carboxy, formyl, phosphonoxy, or phosphono; or -L_{S}-R_{E};
R_{B} and R_{B}′ are each independently selected at each occurrence from hydrogen; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆carbocycle or 3- to 6-membered heterocycle; or C₃-C₆carbocycle or 3- to 6-membered heterocycle; wherein each C₃-C₆carbocycle or 3- to 6-membered heterocycle in R_{B} or R_{B}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′) or —C(O)N(R_{S})C(O)—R_{S}′; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{L} is independently selected at each occurrence from halogen, nitro, oxo, phosphonoxy, phosphono, thioxo, cyano, —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′) or —N(R_{S})C(O)R_{S}′; or C₃-C₆carbocycle 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{S}, R_{S}′ and R_{S}″ are each independently selected at each occurrence from hydrogen; C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano or 3- to 6-membered carbocycle or heterocycle; or 3- to 6-membered carbocycle or heterocycle; wherein each 3- to 6-membered carbocycle or heterocycle in R_{S}, R_{S}′ or R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

24. The NS5A inhibitor of claim 23, wherein said moiety comprises C₅-C₆carbocycle, 5- to 6-membered heterocycle, or 6- to 12-membered bicycle, each of which is optionally substituted with one or more R_{A}.
